(19) United States
(12) Patent Application Publication
(10) Pub. No.: US 2011/0064743 A1
(43) Pub. Date: Mar. 17, 2011

(54) HUMAN ANTI-CANCER ANTIBODIES

(76) Inventors: PHILIP W. HAMMOND, SEATTLE, WA (US); ANNE-RENEE VAN DER VUURST DE VRIES, PREVESSIN (FR); LARRY W. TJOELKER, KIRKLAND, WA (US); MATTHEW MOYLE, REDMOND, WA (US)

(21) Appl. No.: 12/878,665

(22) Filed: Sep. 9, 2010

Related U.S. Application Data
(60) Provisional application No. 61/240,827, filed on Sep. 9, 2009.

(51) Int. Cl. A61K 39/395; C07K 16/30; G01N 33/53; A61P 35/00

(52) U.S. Cl. 424/142.1; 530/388.15; 530/389.7; 424/174.1; 436/501

(57) ABSTRACT

The present invention provides novel human anti-cancer antibodies and related compositions and methods. These antibodies are used in the diagnosis and treatment of cancer.

### RELATED APPLICATIONS

This application claims the benefit of provisional application U.S. Ser. No. 61/240,827, filed Sep. 9, 2009, the contents of which are herein incorporated by reference in their entirety.

### INCORPORATION OF SEQUENCE LISTING

The contents of the text file named “37418-511001USSeqList.txt,” which was created on Sep. 7, 2010 and is 49.6 KB in size, are hereby incorporated by reference in their entirety.

### FIELD OF THE INVENTION

The present invention relates generally to therapy, diagnosis and monitoring of cancer. The invention is more specifically related to anti-cancer cell antibodies and their manufacture and use. Such antibodies are useful in pharmaceutical compositions for the prevention and treatment of cancer and for the diagnosis and monitoring of cancer.

### BACKGROUND OF THE INVENTION

Cancer is a significant health problem throughout the world. Although advances have been made in detection and therapy of cancer, no vaccine or other universally successful method for prevention or treatment is currently available. Current therapies, which are generally based on a combination of chemotherapy or surgery and radiation, continue to prove inadequate in many patients.

More recently, efforts have been made to harness the body's natural defense system to prevent or treat cancer, e.g., by developing cancer vaccines and therapeutic antibodies. It is hoped that such agents will specifically target tumor cells, thereby reducing the damaging side effects of many current cancer treatments on healthy tissue. Herceptin® is the first humanized antibody approved for the treatment of HER2 positive metastatic breast cancer. Herceptin® is designed to target and block the function of HER2 protein overexpression associated with a specific, aggressive form of breast cancer. The Rituxan® (Rituximab) antibody is another example of a promising tumor-specific therapeutic antibody. Rituxan® is a genetically engineered chimeric murine/human monoclonal antibody directed against the CD20 antigen found on the surface of normal and malignant B lymphocytes. Rituxan® causes lysis of the B lymphocytes by activating the complement cascade and immune effector cells (antibody-dependent cell-mediated cytotoxicity), and inducing apoptosis. Both of these antibodies have proven effective in treating specific forms of cancer.

However, in spite of considerable research into therapies for cancer and the more recent developments in cancer immunotherapeutics, most cancers remain difficult to diagnose and treat effectively.

Accordingly, there is a need in the art for improved methods for detecting and treating cancers. The present invention fulfills these needs and further provides other related advantages.

### SUMMARY OF THE INVENTION

The present invention provides fully human monoclonal antibodies specifically directed against cancer antigens. Optionally, the antibody is isolated form a B-cell from a human donor. Exemplary monoclonal antibodies include, but are not limited to, 1061_I16 (also designated as TCN-462), 1226_K16, 1242_P11, 1242_N12, 1256_B2, 1250_I13, 1252_B7, 1248_C17, 1247_A18, 1252_O13, 1038_D5 (also designated as TCN-445), and 1261_P5, described herein. Alternatively, the monoclonal antibody is an antibody that binds to the same epitope as 1061_I16 (TCN-462), 1226_K16, 1242_P11, 1242_N12, 1256_B2, 1250_I13, 1252_B7, 1248_C17, 1247_A18, 1252_O13, 1038_D5 (TCN-445), and 1261_P5. The antibodies are respectively referred to herein as huCA antibodies.

A huCA antibody contains a heavy chain variable having the amino acid sequence of SEQ ID NOS: 1, 5, 9, 13, 17, 21, 25, 29, 33, 37, 41, or 45 and/or a light chain variable region amino acid of SEQ ID NO: 3, 7, 11, 15, 19, 23, 27, 31, 35, 39, 43, or 47.

In one aspect, the three heavy chain CDRs include an amino acid sequence at least 90%, 92%, 95%, 97% 98%, 99% or more identical to the amino acid sequence of SEQ ID NO: 49, 57, 75, 87, 95, 101, 103, 119, 50, 59, 65, 71, 79, 88, 96, 104, 110, 121 51, 58, 64, 66, 72, 80, 82, 89, 97, 105, or 111, and a light chain with three CDRs that include an amino acid sequence at least 90%, 92%, 95%, 97% 98%, 99% or more identical to the amino acid sequence of SEQ ID NO: 60, 81, 90, 98, 106, 112, 115, 116, 117, 118, 53, 61, 67, 73, 76, 83, 91, 99, 113, 52, 54, 62, 68, 77, 85, 92, 100, 107, or 114.

Alternatively, the three heavy chain CDRs include an amino acid sequence at least 90%, 92%, 95%, 97% 98%, 99% or more identical to the amino acid sequence of SEQ ID NO: 55, 63, 69, 84, 86, 93, 101, 108, 56, 65, 70, 74, 78, 94, 102, 109, 120, 51, 58, 64, 66, 72, 80, 82, 89, 97, 105, or 111, and a light chain with three CDRs that include an amino acid sequence at least 90%, 92%, 95%, 97% 98%, 99% or more identical to the amino acid sequence of SEQ ID NO: 60, 81, 90, 98, 106, 112, 115, 116, 117, 118, 3, 61, 67, 73, 76, 83, 91, 99, 113, 52, 54, 62, 68, 77, 85, 92, 100, 107, or 114.

The invention provides an isolated fully human monoclonal anti-cancer antibody or fragment thereof, wherein said antibody includes: (a) a V_{H} CDR1 region including the amino acid sequence of SEQ ID NO: 49, 57, 75, 87, 95, 101, 103, or 119; (b) a V_{H} CDR2 region including the amino acid sequence of SEQ ID NO: 50, 59, 65, 71, 79, 88, 96, 104, 110 or 121; and (c) a V_{H} CDR3 region including the amino acid sequence of SEQ ID NO: 51, 58, 64, 66, 72, 80, 82, 89, 97, 105, or 111. Optionally, this antibody further includes: (a) a V_{L} CDR1 region including the amino acid sequence of SEQ ID NO: 60, 81, 90, 98, 106, 112, 115, 116, 117 or 118; (b) a V_{L} CDR2 region including the amino acid sequence of SEQ ID NO: 53, 61, 67, 73, 76, 83, 91, 99, or 113; and (c) a V_{L} CDR3 region including the amino acid sequence of SEQ ID NO: 52, 54, 62, 68, 77, 85, 92, 100, 107, or 114.

The invention provides an isolated fully human monoclonal anti-cancer antibody or fragment thereof, wherein said antibody includes: (a) a V_{H} CDR1 region comprising the amino acid sequence of SEQ ID NO: 55, 63, 69, 84, 86, 93, 101, or 108; (b) a V_{H} CDR2 region comprising the amino acid sequence of SEQ ID NO: 56, 65, 70, 74, 78, 94, 102, 109 or 121; and (c) a V_{H} CDR3 region comprising the amino acid sequence of SEQ ID NO: 51, 58, 64, 66, 72, 80, 82, 89, 97, 105, or 111. Optionally, this antibody further includes: (a) a V_{L} CDR1 region comprising the amino acid sequence of SEQ ID NO: 60, 81, 90, 98, 106, 112, 115, 116, 117 or 118; (b) a V_{L} CDR2 region comprising the amino acid sequence of SEQ ID NO: 53, 61, 67, 73, 76, 83, 91, 99, or 113; and (c) a V_{L} CDR3 region comprising the amino acid sequence of SEQ ID NO: 52, 54, 62, 68, 77, 85, 92, 100, 107, or 114.

The invention also provides an isolated fully human monoclonal anti-cancer antibody or fragment thereof including: a) a heavy chain sequence including the amino acid sequence of SEQ ID NO: 1 and a light chain sequence including amino acid sequence SEQ ID NO: 3; b) a heavy chain sequence including the amino acid sequence of SEQ ID NO: 5 and a light chain sequence including amino acid sequence SEQ ID NO: 7; c) a heavy chain sequence including the amino acid sequence of SEQ ID NO: 9 and a light chain sequence including amino acid sequence SEQ ID NO: 11; d) a heavy chain sequence including the amino acid sequence of SEQ ID NO: 13 and a light chain sequence including amino acid sequence SEQ ID NO: 15; e) a heavy chain sequence including the amino acid sequence of SEQ ID NO: 17 and a light chain sequence including amino acid sequence SEQ ID NO: 19; f) a heavy chain sequence including the amino acid sequence of SEQ ID NO: 21 and a light chain sequence including amino acid sequence SEQ ID NO: 23; g) a heavy chain sequence including the amino acid sequence of SEQ ID NO: 25 and a light chain sequence including amino acid sequence SEQ ID NO: 27; h) a heavy chain sequence including the amino acid sequence of SEQ ID NO: 29 and a light chain sequence including amino acid sequence SEQ ID NO: 31; i) a heavy chain sequence including the amino acid sequence of SEQ ID NO: 33 and a light chain sequence including amino acid sequence SEQ ID NO: 35; j) a heavy chain sequence including the amino acid sequence of SEQ ID NO: 37 and a light chain sequence including amino acid sequence SEQ ID NO: 39; k) a heavy chain sequence including the amino acid sequence of SEQ ID NO: 41 and a light chain sequence including amino acid sequence SEQ ID NO: 43; or 1) a heavy chain sequence including the amino acid sequence of SEQ ID NO: 45 and a light chain sequence including amino acid sequence SEQ ID NO: 47.

The invention provides an isolated anti-cancer antibody, wherein the antibody has a heavy chain with three CDRs including an amino acid sequence selected from the group consisting of the amino acid sequences of SGYYWS (SEQ ID NO: 49), EINHSGSTNYNPSLKS (SEQ ID NO: 50) and GGGRAGGSCCIRRPREYFQH (SEQ ID NO: 51), and a light chain with three CDRs that include an amino acid sequence selected from the group consisting of the amino acid sequences of KSSQSVLYSSNNKNYLA (SEQ ID NO: 115), WASTRES (SEQ ID NO: 53) and QQYYSTPPRT (SEQ ID NO: 54).

The invention provides an isolated anti-cancer antibody, wherein said antibody has a heavy chain with three CDRs including an amino acid sequence selected from the group consisting of the amino acid sequences of GGSFSG (SEQ ID NO: 55), EINHSGSTN (SEQ ID NO: 56) and GGGRAGGSCCIRRPREYFQH (SEQ ID NO: 51) and a light chain with three CDRs that include an amino acid sequence selected from the group consisting of the amino acid sequences of KSSQSVLYSSNNKNYLA (SEQ ID NO: 115), WASTRES (SEQ ID NO: 53) and QQYYSTPPRT (SEQ ID NO: 54).

The invention provides an isolated anti-cancer antibody, wherein said antibody has a heavy chain with three CDRs including an amino acid sequence selected from the group consisting of the amino acid sequences of GYFWT (SEQ ID NO: 57), EINHRRTTTSNPSLRS (SEQ ID NO: 59) and ITEAVGVTSFDY (SEQ ID NO: 58), and a light chain with three CDRs that include an amino acid sequence selected from the group consisting of the amino acid sequences of SGSTSNIGNNFVA (SEQ ID NO: 60), DNDKRPS (SEQ ID NO: 61) and GTWDSTLSRV (SEQ ID NO: 62).

The invention provides an isolated anti-cancer antibody, wherein said antibody has a heavy chain with three CDRs including an amino acid sequence selected from the group consisting of the amino acid sequences of GGSLSG (SEQ ID NO: 63), EINHRRTTT (SEQ ID NO: 65) and ITEAVGVTSFDY (SEQ ID NO: 58), and a light chain with three CDRs that include an amino acid sequence selected from the group consisting of the amino acid sequences of SGSTSNIGNNFVA (SEQ ID NO: 60), DNDKRPS (SEQ ID NO: 61) and GTWDSTLSRV (SEQ ID NO: 62).

The invention provides an isolated anti-cancer antibody, wherein said antibody has a heavy chain with three CDRs including an amino acid sequence selected from the group consisting of the amino acid sequences of GYFWT (SEQ ID NO: 57), EINHKGKTTYNPTLKS (SEQ ID NO: 65) and IVEAVGVTSFDS (SEQ ID NO: 66), and a light chain with three CDRs that include an amino acid sequence selected from the group consisting of the amino acid sequences of SGSTSNIGNNHVS (SEQ ID NO: 116), DNNKRPS (SEQ ID NO: 67) and GTWDTRLSRV (SEQ ID NO: 68).

The invention provides an isolated anti-cancer antibody, wherein said antibody has a heavy chain with three CDRs including an amino acid sequence selected from the group consisting of the amino acid sequences of GGGSFSG (SEQ ID NO: 69), EINHKGKTT (SEQ ID NO: 70) and IVEAVGVTSFDS (SEQ ID NO: 66), and a light chain with three CDRs that include an amino acid sequence selected from the group consisting of the amino acid sequences of SGSTSNIGNNHVS (SEQ ID NO: 116), DNNKRPS (SEQ ID NO: 67) and GTWDTRLSRV (SEQ ID NO: 68).

The invention provides an isolated anti-cancer antibody, wherein said antibody has a heavy chain with three CDRs including an amino acid sequence selected from the group consisting of the amino acid sequences of GYFWT (SEQ ID NO: 57), EINHRGSSSYNPSLRS (SEQ ID NO: 71) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DDDKRPS (SEQ ID NO: 73) and GTWDSSLSRV (SEQ ID NO: 52).

The invention provides an isolated anti-cancer antibody, wherein said antibody has a heavy chain with three CDRs including an amino acid sequence selected from the group consisting of the amino acid sequences of GGSFSG (SEQ ID NO: 55), EINHRGSSS (SEQ ID NO: 74) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DDDKRPS (SEQ ID NO: 73) and GTWDSSLSRV (SEQ ID NO: 52).

The invention provides an isolated anti-cancer antibody, wherein said antibody has a heavy chain with three CDRs including an amino acid sequence selected from the group consisting of the amino acid sequences of GYFWS (SEQ ID NO: 75), EINHRGSSTYKSSLKT (SEQ ID NO: 121) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DNDKRPS (SEQ ID NO: 61) and GTWDNNLSRV (SEQ ID NO: 77).

The invention provides an isolated anti-cancer antibody, wherein said antibody has a heavy chain with three CDRs including an amino acid sequence selected from the group consisting of the amino acid sequences of GGSFSG (SEQ ID NO: 55), EINHRGSST (SEQ ID NO: 78) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DNDKRPS (SEQ ID NO: 61) and GTWDNNLSRV (SEQ ID NO: 77).

The invention provides an isolated anti-cancer antibody, wherein said antibody has a heavy chain with three CDRs including an amino acid sequence selected from the group consisting of the amino acid sequences of GYFWT (SEQ ID NO: 57), EINHRGTSS (SEQ ID NO: 79) and ITEAVGFTSFDY (SEQ ID NO: 80), and a light chain with three CDRs that include an amino acid sequence selected from the group consisting of the amino acid sequences of SGSTSNIGSNYVS (SEQ ID NO: 118), DNDKRPS (SEQ ID NO: 61) and GTWDSSLSRV (SEQ ID NO: 52).

The invention provides an isolated anti-cancer antibody, wherein said antibody has a heavy chain with three CDRs including an amino acid sequence selected from the group consisting of the amino acid sequences of GGSFSG (SEQ ID NO: 55), EINHRGTSS (SEQ ID NO: 79) and ITEAVGFTSFDY (SEQ ID NO: 80), and a light chain with three CDRs that include an amino acid sequence selected from the group consisting of the amino acid sequences of SGSTSNIGSNYVS (SEQ ID NO: 118), DNDKRPS (SEQ ID NO: 61) and GTWDSSLSRV (SEQ ID NO: 52).

The invention provides an isolated anti-cancer antibody, wherein said antibody has a heavy chain with three CDRs including an amino acid sequence selected from the group consisting of the amino acid sequences of GYFWS (SEQ ID NO: 75), EINHSGSTNYNPSLKS (SEQ ID NO: 50) and GMVVAGTRSDAFDI (SEQ ID NO: 64), and a light chain with three CDRs that include an amino acid sequence selected from the group consisting of the amino acid sequences of SGSSSNIGINTVN (SEQ ID NO: 81), SNNQRPS (SEQ ID NO: 76) and AAWDDSLNEV (SEQ ID NO: 85).

The invention provides an isolated anti-cancer antibody, wherein said antibody has a heavy chain with three CDRs including an amino acid sequence selected from the group consisting of the amino acid sequences of IGSFRG (SEQ ID NO: 86), EINHSGSTN (SEQ ID NO: 56) and GMVVAGTRSDAFDI (SEQ ID NO: 64), and a light chain with three CDRs that include an amino acid sequence selected from the group consisting of the amino acid sequences of SGSSSNIGINTVN (SEQ ID NO: 81), SNNQRPS (SEQ ID NO: 76) and AAWDDSLNEV (SEQ ID NO: 85).

The invention provides an isolated anti-cancer antibody, wherein said antibody has a heavy chain with three CDRs including an amino acid sequence selected from the group consisting of the amino acid sequences of GYFWS (SEQ ID NO: 75), EINHSGSTNYNPSLKS (SEQ ID NO: 50) and GIVVAGTRSDAFDI (SEQ ID NO: 82), and a light chain with three CDRs that include an amino acid sequence selected from the group consisting of the amino acid sequences of SGSSSNIGINTVN (SEQ ID NO: 81), NNNQRPS (SEQ ID NO: 83) and AAWDDSLNEV (SEQ ID NO: 85).

The invention provides an isolated anti-cancer antibody, wherein said antibody has a heavy chain with three CDRs including an amino acid sequence selected from the group consisting of the amino acid sequences of IGSFRG (SEQ ID NO: 86), EINHSGSTN (SEQ ID NO: 56) and GIVVAGTRSDAFDI (SEQ ID NO: 82), and a light chain with three CDRs that include an amino acid sequence selected from the group consisting of the amino acid sequences of SGSSSNIGINTVN (SEQ ID NO: 81), NNNQRPS (SEQ ID NO: 83) and AAWDDSLNEV (SEQ ID NO: 85).

The invention provides an isolated anti-cancer antibody, wherein said antibody has a heavy chain with three CDRs including an amino acid sequence selected from the group consisting of the amino acid sequences of SYWMN (SEQ ID NO: 87), NINQDGTEKNYVDSVKG (SEQ ID NO: 88) and GVFQGAPHFVF (SEQ ID NO: 89), and a light chain with three CDRs that include an amino acid sequence selected from the group consisting of the amino acid sequences of RSSQSLLHGNGFNYLD (SEQ ID NO: 90), LGSDRAS (SEQ ID NO: 91) and MQSLRTPLT (SEQ ID NO: 92).

The invention provides an isolated anti-cancer antibody, wherein said antibody has a heavy chain with three CDRs including an amino acid sequence selected from the group consisting of the amino acid sequences of EFTFGS (SEQ ID NO: 93), NINQDGTEKN (SEQ ID NO: 94) and GVFQGAPHFVF (SEQ ID NO: 89), and a light chain with three CDRs that include an amino acid sequence selected from the group consisting of the amino acid sequences of RSSQSLLHGNGFNYLD (SEQ ID NO: 90), LGSDRAS (SEQ ID NO: 91) and MQSLRTPLT (SEQ ID NO: 92).

The invention provides an isolated anti-cancer antibody, wherein said antibody has a heavy chain with three CDRs including an amino acid sequence selected from the group consisting of the amino acid sequences of RYDIS (SEQ ID NO: 95), WMNPNSGNTGYAQKFQD (SEQ ID NO: 96) and LRVESLGRRFFYAYNGMDV (SEQ ID NO: 97), and a light chain with three CDRs that include an amino acid sequence selected from the group consisting of the amino acid sequences of QASQDISNYLN (SEQ ID NO: 98), DASNLET (SEQ ID NO: 99) and QQYNNVLFT (SEQ ID NO: 100).

The invention provides an isolated anti-cancer antibody, wherein said antibody has a heavy chain with three CDRs including an amino acid sequence selected from the group consisting of the amino acid sequences of GYTFNR (SEQ ID NO: 101), WMNPNSGNTG (SEQ ID NO: 102) and LRVESLGRRFFYAYNGMDV (SEQ ID NO: 97), and a light chain with three CDRs that include an amino acid sequence selected from the group consisting of the amino acid sequences of QASQDISNYLN (SEQ ID NO: 98), DASNLET (SEQ ID NO: 99) and QQYNNVLFT (SEQ ID NO: 100).

The invention provides an isolated anti-cancer antibody, wherein said antibody has a heavy chain with three CDRs including an amino acid sequence selected from the group consisting of the amino acid sequences of DFYFH (SEQ ID NO: 103), WINPRSGATNYAHKFRG (SEQ ID NO: 104) and DMRRENGYNFDGTFDY (SEQ ID NO: 105), and a light chain with three CDRs that include an amino acid sequence selected from the group consisting of the amino acid sequences of QASQDIKNYLN (SEQ ID NO: 106), DASNLET (SEQ ID NO: 99) and QRYDAFPLT (SEQ ID NO: 107).

The invention provides an isolated anti-cancer antibody, wherein said antibody has a heavy chain with three CDRs including an amino acid sequence selected from the group consisting of the amino acid sequences of GYTFTD (SEQ ID NO: 108), WINPRSGATN (SEQ ID NO: 109) and DMRRENGYNFDGTFDY (SEQ ID NO: 105), and a light chain with three CDRs that include an amino acid sequence selected from the group consisting of the amino acid sequences of QASQDIKNYLN (SEQ ID NO: 106), DASNLET (SEQ ID NO: 99) and QRYDAFPLT (SEQ ID NO: 107).

The invention provides an isolated anti-cancer antibody, wherein said antibody has a heavy chain with three CDRs including an amino acid sequence selected from the group consisting of the amino acid sequences of SYWMS (SEQ ID NO: 119), NIKQDGSEKYYVDSVKG (SEQ ID NO: 110) and DSEVAAAGTHFHY (SEQ ID NO: 111), and a light chain with three CDRs that include an amino acid sequence selected from the group consisting of the amino acid sequences of RASQSISTYLN (SEQ ID NO: 112), AASSLQS (SEQ ID NO: 113) and QQSYTALT (SEQ ID NO: 114).

The invention provides an isolated anti-cancer antibody, wherein said antibody has a heavy chain with three CDRs including an amino acid sequence selected from the group consisting of the amino acid sequences of GFSFSS (SEQ ID NO: 84), NIKQDGSEKY (SEQ ID NO: 120) and DSEVAAAGTHFHY (SEQ ID NO: 111), and a light chain with three CDRs that include an amino acid sequence selected from the group consisting of the amino acid sequences of RASQSISTYLN (SEQ ID NO: 112), AASSLQS (SEQ ID NO: 113) and QQSYTALT (SEQ ID NO: 114).

The invention provides an antibody that binds the same epitope as monoclonal antibody 1061_I16 (TCN-462), 1226_K16, 1242_P11, 1242_N12, 1256_B2, 1250_I13, 1252_B7, 1248_C17, 1247_A18, 1252_O13, 1038_D5 (TCN-445), or 1261_P5.

In another aspect the invention provides a composition including a huCA antibody according to the invention. Optionally, the composition is a pharmaceutical composition that includes a huCA antibody according to the invention and a pharmaceutical carrier. In various aspects, the composition further includes a second anti-cancer antibody or an anti-cancer drug, e.g. a chemotherapeutic agent. The second anti-cancer antibody is optionally a huCA antibody according to the invention.

In a further aspect the huCA antibodies according to the invention are operably-linked to a therapeutic agent or a detectable label.

Additionally, the invention provides methods of treating, preventing or alleviating a symptom of a cancer by administering a huCA antibody to a subject. The cancer is, for example, breast cancer or ovarian cancer. The subject either has been diagnosed with cancer or has not been diagnosed with cancer. Alternatively, the subject has an increased risk of developing cancer due to exposure to a carcinogen (e.g. radiation, mutagen, or virus (AIDS, HPV, etc.)) or a genetic predisposition to developing cancer (e.g. carries a mutation in the BRCA1 or BRCA2 gene or has at least one blood relative who has been diagnosed with a form of cancer, for example).

Optionally, the subject is further administered with a second agent including, but not limited to, a second anti-cancer antibody or an anti-cancer drug, e.g. a chemotherapeutic agent. The second anti-cancer antibody or the anti-cancer drug is administered simultaneously or sequentially with a huCA of the invention. For example, the second anti-cancer antibody or the anti-cancer drug is administered before or after a huCA of the invention.

In another aspect, the invention provides methods of administering the huCA antibody of the invention to a subject prior to, during the development of cancer, and/or after developing cancer.

Also included in the invention is a method for determining the presence of a cancer in a patient, by contacting a biological sample obtained from the patient with a huCA antibody of the invention or a composition of the invention; detecting an amount of the antibody that binds to the biological sample; and comparing the amount of antibody that binds to the biological sample to a control value.

Cancers include, but are not limited to, acute lymphoblastic leukemia, acute myeloid leukemia, adrenocortical carcinoma, AIDS-related cancers, AIDS-related lymphoma, anal cancer, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, mycosis fungoides, Sézary Syndrome, endometrial cancer, esophageal cancer, extracranial germ cell tumor, extragonadal germ cell tumor, eye cancer, tetraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, islet cell tumors (endocrine pancreas), Kaposi Sarcoma, kidney (renal cell) cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, non-small cell lung cancer, small cell lung cancer, non-Hodgkin lymphoma, primary central nervous system lymphoma, Waldenström macroglobulinemia, medulloblastoma, melanoma, intraocular (eye) melanoma, merkel cell carcinoma, mesothelioma malignant, mesothelioma, metastatic squamous neck cancer, mouth cancer, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine sarcoma, skin cancer (nonmelanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilm's Tumor.

The invention further provides a diagnostic kit including a huCA antibody according to the invention.

The invention also provides a prophylactic kit including an epitope of an antibody according to the invention or an antibody according to the invention.

Other features and advantages of the invention will be apparent from and are encompassed by the following detailed description and claims.

### BRIEF DESCRIPTION OF THE DRAWINGS

FIG. 1 is a series of graphs showing the reactivity of sera obtained from subjects with (bottom) or without (top) ovarian cancer against various tumor cell lines.

FIG. 2 is a chart showing cell line reactivity signatures for serum samples from breast and ovarian cancer patients.

FIG. 3 is a series of charts showing the serological profile of monoclonal antibodies recovered from donor N-041 recapitulate the profile seen the serum.

FIG. 4 is a series of charts showing that the serological profile of monoclonal antibody 1038_D5 (TCN-445) recovered from donor F-018 recapitulates the profile seen the serum.

FIG. 5 is a series of charts showing that the serological profile of monoclonal antibody 1061_I16 (TCN-462) recovered from donor F-017 recapitulates the profile seen from the serum.

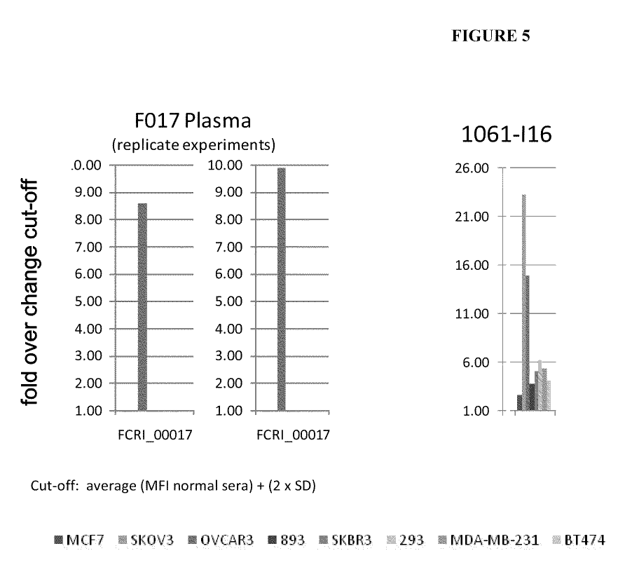

FIG. 6A-B is a pair of graphs showing the data used for the affinity determination of monoclonal antibody 1038_D5 (TCN-445) to OVCAR-3 cells.

FIG. 7 is a chart showing that monoclonal antibody 1038_D5 (TCN-445) mediates ADCC.

FIG. 8 is a pair of photographs depicting immunohistochemistry of normal ovary and ovarian carcinoma with 1038_D5 (TCN-445).

FIG. 9 is a series of graphs depicting the results of Fluorescence Activated Cell Sorting (FACS) experiments in which cancer cells (A-673, A2780, ARH-787, Calu-6, FaDu, HCT-116, Hs746T, HT-29, LoVo, LS174T, MCF-7, MX-1 tumor, NIX:OVCAR-3 theraclone, NIH:OVCAR-3 ODS, SU-DHL-4, and U-87 MG cells) are bound to either 2N9 (also designated as TCN-202), or 1038_D5 (TCN-445), or a positive control, anti-hIgG, each conjugated to a fluorescent tag (FITC). Antibody binding to each cancer cell type is demonstrated as a function of log fluorescence of the signal versus the percentage (%) of total cells analyzed. The results show that both 2N9 (TNC-202) and 1038_D5 (TCN-445) bind all types of cancer cells.

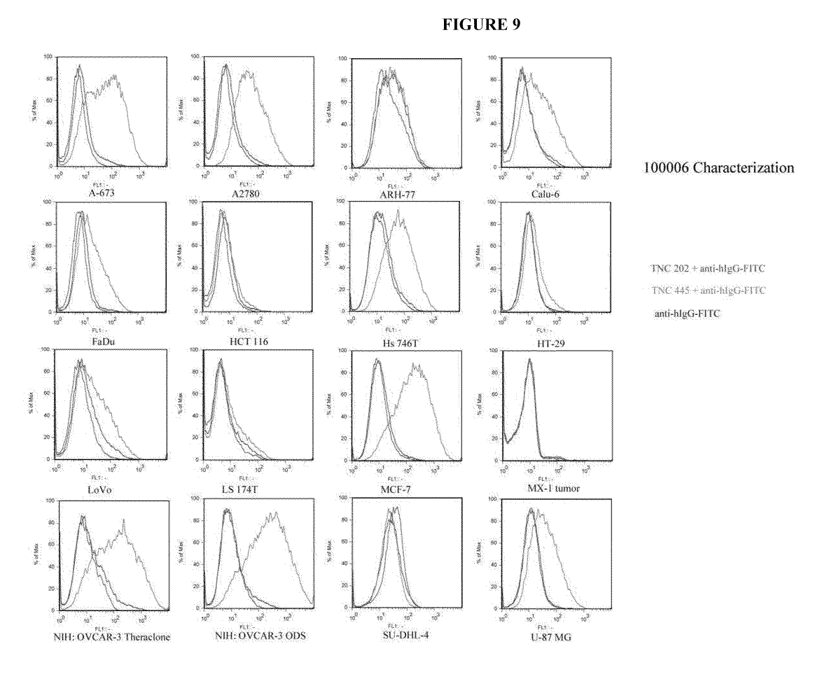

FIG. 10 is a series of photographs depicting immunohistochemistry performed on a sample harvested from the normal colon of a first human adult donor. The 1038_D5 (TCN-445) monoclonal antibody or the isotype-2N9 antibody was applied to tissue sections.

FIG. 11 is a series of photographs depicting immunohistochemistry performed on a sample harvested from the normal colon of a second human adult donor. The 1038_D5 (TCN-445) monoclonal antibody or the isotype-2N9 antibody was applied to tissue sections.

FIG. 12 is a series of photographs depicting immunohistochemistry performed on a sample harvested from the normal colon of a third human adult donor. The 1038_D5 (TCN-445) monoclonal antibody or the isotype-2N9 antibody was applied to tissue sections.

FIG. 13 is a series of photographs depicting immunohistochemistry performed on a sample harvested from the normal small intestine of a first human adult donor. The 1038_D5 (TCN-445) monoclonal antibody or the isotype-2N9 antibody was applied to tissue sections.

FIG. 14 is a series of photographs depicting immunohistochemistry performed on a sample harvested from the normal small intestine of a second human adult donor. The 1038_D5 (TCN-445) monoclonal antibody or the isotype-2N9 antibody was applied to tissue sections.

FIG. 15 is a series of photographs depicting immunohistochemistry performed on a sample harvested from the normal small intestine of a third human adult donor. The 1038_D5 (TCN-445) monoclonal antibody or the isotype-2N9 antibody was applied to tissue sections.

FIG. 16 is a tissue array diagram depicting the microarray used for the FDA Standard Frozen Tissue Array described herein.

FIG. 17 is a representative image of a normal frozen tissue sample from the FDA Tissue Microarray study conducted herein. These images show sections of the adrenal gland on which immunohistochemistry experiments were performed using either the 1038_D5 (TCN-445) monoclonal antibody or the isotype-2N9 control.

FIG. 18 is a representative image of a normal frozen tissue sample from the FDA Tissue Microarray study conducted herein. These images show sections of the cerebellum of the brain on which immunohistochemistry experiments were performed using either the 1038_D5 (TCN-445) monoclonal antibody or the isotype-2N9 control.

FIG. 19 is a representative image of a normal frozen tissue sample from the FDA Tissue Microarray study conducted herein. These images show sections of the colon on which immunohistochemistry experiments were performed using either the 1038_D5 (TCN-445) monoclonal antibody or the isotype-2N9 control.

FIG. 20 is a representative image of a normal frozen tissue sample from the FDA Tissue Microarray study conducted herein. These images show sections of the placenta on which immunohistochemistry experiments were performed using either the 1038_D5 (TCN-445) monoclonal antibody or the isotype-2N9 control.

FIG. 21 is a representative image of a normal frozen tissue sample from the FDA Tissue Microarray study conducted herein. These images show sections of the skeletal muscle myocytes on which immunohistochemistry experiments were performed using either the 1038_D5 (TCN-445) monoclonal antibody or the isotype-2N9 control.

FIG. 22 is a representative image of a normal frozen tissue sample from the FDA Tissue Microarray study conducted herein. These images show sections through the skin, epidermis, and dermis on which immunohistochemistry experiments were performed using either the 1038_D5 (TCN-445) monoclonal antibody or the isotype-2N9 control.

FIG. 23 is a representative image of a normal frozen tissue sample from the FDA Tissue Microarray study conducted herein. These images show sections of the small intestine on which immunohistochemistry experiments were performed using either the 1038_D5 (TCN-445) monoclonal antibody or the isotype-2N9 control.

### DETAILED DESCRIPTION

The present invention provides fully human monoclonal antibodies specific against cancer. The antibodies are respectively referred to herein as huCA antibodies. The fully huCA antibodies were identified by screening various cancer cell lines against antibodies derived from cultured B cells obtained from ovarian or breast cancer patients.

The 1061_I16 (TCN-462) antibody (also referred to herein as 116) includes a heavy chain variable region (SEQ ID NO:1) encoded by the nucleic acid sequence shown below in SEQ ID NO: 2, and a light chain variable region (SEQ ID NO: 3) encoded by the nucleic acid sequence shown in SEQ ID NO: 4.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the 116 antibody have the following sequences per Kabat definition: CDR1, SGYYWS (SEQ ID NO: 49), CDR2, EINHSGSTNYNPSLKS (SEQ ID NO: 50) and CDR3, GGGRAGGSCCIRRPREYFQH (SEQ ID NO: 51). The light chain CDRs of the 116 antibody have the following sequences per Kabat definition: CDR1, KSSQSVLYSSNNKNYLA (SEQ ID NO: 115), CDR2, WASTRES (SEQ ID NO: 53) and CDR3, QQYYSTPPRT (SEQ ID NO: 54).

The heavy chain CDRs of the 116 antibody have the following sequences per Chothia definition: CDR1, GGSFSG (SEQ ID NO: 55), CDR2, EINHSGSTN (SEQ ID NO: 56) and CDR3, GGGRAGGSCCIRRPREYFQH (SEQ ID NO: 51). The light chain CDRs of the 116 antibody have the following sequences per Chothia definition: CDR1, KSSQSVLYSSNNKNYLA (SEQ ID NO: 115), CDR2, WASTRES (SEQ ID NO: 53) and CDR3, QQYYSTPPRT (SEQ ID NO: 54).

1061_I16 (TCN-462) VH nucleotide sequence
(SEQ ID NO: 2)
CAGGTGCAGCTACAGCAGTGGGGCGCAGGACTGTTGAAGCCTTCGGAG
ACCCTGTCCCTCACCTGCGCTGTCTATGGTGGGTCCTTCAGTGGTTAC
TACTGGAGCTGGATCCGCCAGCCCCCAGGGAAGGGGCTGGAGTGGATT
GGGGAAATCAATCATAGTGGAAGCACCAACTACAACCCGTCCCTCAAG
AGTCGAGTCACCATATCAGTAGACACGTCCAAGAACCAGTTCTCCCTG
AAGCTGAGCTCTGTGACCGCCGCGGACACGGCTGTGTATTACTGTGCG
AGAGGCGGGGGTAGGGCTGGTGGTAGCTGCTGTATCCGACGCCCCCGA
GAATACTTCCAGCACTGGGGCCAGGGCACCCTGGTCACCGTCTCCTCA
1061_I16 (TCN-462) VH amino acid sequence
(Kabat CDRS in bold, Chothia CDRs in underline):
(SEQ ID NO: 1)
QLQQWGAGLLKPSETLSLTCAVYGGSFSGYYWSWIRQPPGKGLEWIG
EINHSGSTNYNPSLKSRVTISVDTSKNQFSLKLSSVTAADTAVYYCA
RGGGRAGGSCCIRRPREYFQHWGQGTLVTVSS
1061_I16 (TCN-462) VL nucleotide sequence
(SEQ ID NO: 4)
GACATCGTGATGACCCAGTCTCCAGACTCCCTGGCTGTGTCTCTGGGC
GAGAGGGCCACCATCAACTGCAAGTCCAGCCAGAGTGTTTTATACAGC
TCCAACAATAAGAACTACTTAGCTTGGTACCAGCAGAAACCAGGACAG
CCTCCTAAGCTGCTCATTTACTGGGCATCTACCCGGGAATCCGGGGTC
CCTGACCGATTCAGTGGCAGCGGGTCTGGGACAGATTTCACTCTCACC
ATCAGCAGCCTGCAGGCTGAAGATGTGGCAGTTTATTACTGTCAGCAA
TATTATAGTACTCCTCCGAGGACTTTTGGCCAGGGGACCAAGCTGGAG
ATCAAA
1061_I16 (TCN-462) VL amino acid sequence
(Kabat CDRS in bold, Chothia CDRs in underline):
(SEQ ID NO: 3)
DIVMTQSPDSLAVSLGERATINCKSSQSVLYSSNNKNYLAWYQQKPGQ
PPKLLIYWASTRESGVPDRFSGSGSGTDFTLTISSLQAEDVAVYYC
QQYYSTPPRTFGQGTKLEIK

The 1226_K16 antibody (also referred to herein as K16) includes a heavy chain variable region (SEQ ID NO: 5) encoded by the nucleic acid sequence shown below in SEQ ID NO: 6, and a light chain variable region (SEQ ID NO: 7) encoded by the nucleic acid sequence shown in SEQ ID NO: 8.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the K16 antibody have the following sequences per Kabat definition: CDR1, GYFWT (SEQ ID NO: 57), CDR2, EINHRRTTTSNPSLRS (SEQ ID NO: 59) and CDR3, ITEAVGVTSFDY (SEQ ID NO: 58). The light chain CDRs of the K16 antibody have the following sequences per Kabat definition: CDR1, SGSTSNIGNNFVA (SEQ ID NO: 60), CDR2, DNDKRPS (SEQ ID NO: 61) and CDR3, GTWDSTLSRV (SEQ ID NO: 62).

The heavy chain CDRs of the K16 antibody have the following sequences per Chothia definition: CDR1, GGSLSG (SEQ ID NO: 63), CDR2, EINHRRTTT (SEQ ID NO: 65) and CDR3, ITEAVGVTSFDY (SEQ ID NO: 58). The light chain CDRs of the K16 antibody have the following sequences per Chothia definition: CDR1, SGSTSNIGNNFVA (SEQ ID NO: 60), CDR2, DNDKRPS (SEQ ID NO: 61) and CDR3, GTWDSTLSRV (SEQ ID NO: 62).

1226_K16 VH nucleotide sequence
(SEQ ID NO: 6)
CAGGTACAGCTACAACAGTGGGGCGCAGGACTGTTGAAGCCCTCGGAG
ACCCTGTCCCTCACCTGCGCTGTCTATGGTGGGTCCTTAAGTGGTTAT
TTCTGGACTTGGATCCGCCAGCCCCCAGGGAGGGGACTGGAGTGGATT
GGGGAAATCAATCACAGACGAACTACCACCTCCAACCCGTCTCTCAGG
AGTCGACTAACCATATCACTAGACACGTCCAAGAACCAGTTTTCCCTA
AAGCTGAGTTCTGTGACCGCCGCGGACACGGCTGTTTATTATTGTGCG
AGAATTACGGAGGCGGTGGGGGTTACCTCGTTTGACTACTGGGGCCAG
GGAATCCTGGTCACCGTCTCGAGC
1226_K16 VH amino acid sequence (Kabat CDRS in
bold, Chothia CDRs in underline):
(SEQ ID NO: 5)
QVQLQQWGAGLLKPSETLSLTCAVYGGSLSGYFWTWIRQPPGRGLEWI
GEINHRRTTTSNPSLRSRLTISLDTSKNQFSLKLSSVTAADTAVYYCA
RITEAVGVTSFDYWGQGILVTVSS
1226_K16 VL nucleotide sequence
(SEQ ID NO: 8)
CAGTCTGTATTGACGCAGCCGCCCTCAGTGTCTGCGGCCCCAGGACAA
AAGGTCAACATCTCCTGCTCTGGAAGCACCTCCAACATTGGAAATAAT
TTTGTGGCCTGGTACCAGCAACTCCCACAAAGAGCCCCCAAACTCCTC
ATTTACGACAATGATAAGCGACCCTCAGGGATTCCTGACCGATTCTCT
GGCTCCAAGTCCGGCGCGTCAGCCACCCTGGGCATCACCGGACTCCAA
AGTGGGGACGAGGCCTATTATTATTGCGGAACATGGGACAGTACCCTG
AGTCGGGTGTTCGGCGGAGGGACTAAGCTGACCGTTCTA
1226_K16 VL amino acid sequence (Kabat CDRS in
bold, Chothia CDRs in underline):
(SEQ ID NO: 7)
QSVLIQPPSVSAAPGQKVNISCSGSTSNIGNNFVAWYQQLPQRAPKLL
IYDNDKRPSGIPDRFSGSKSGASATLGITGLQSGDEAYYYCGTWDSTL
SRVFGGGTKLTVL

The 1242_P11 antibody (also referred to herein as P11) includes a heavy chain variable region (SEQ ID NO: 9) encoded by the nucleic acid sequence shown below in SEQ ID NO: 10, and a light chain variable region (SEQ ID NO: 11) encoded by the nucleic acid sequence shown in SEQ ID NO: 12.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the P11 antibody have the following sequences per Kabat definition: CDR1, GYFWT (SEQ ID NO: 57), CDR2, EINHKGKTTYNPTLKS (SEQ ID NO: 65) and CDR3, IVEAVGVTSFDS (SEQ ID NO: 66). The light chain CDRs of the P11 antibody have the following sequences per Kabat definition: CDR1, SGSTSNIGNNHVS (SEQ ID NO: 116), CDR2, DNNKRPS (SEQ ID NO: 67) and CDR3, GTWDTRLSRV (SEQ ID NO: 68).

The heavy chain CDRs of the P11 antibody have the following sequences per Chothia definition: CDR1, GGGSFSG (SEQ ID NO: 69), CDR2, EINHKGKTT (SEQ ID NO: 70) and CDR3, IVEAVGVTSFDS (SEQ ID NO: 66). The light chain CDRs of the P11 antibody have the following sequences per Chothia definition: CDR1, SGSTSNIGNNHVS (SEQ ID NO: 116), CDR2, DNNKRPS (SEQ ID NO: 67) and CDR3, GTWDTRLSRV (SEQ ID NO: 68).

1242_P11 VH nucleotide sequence
(SEQ ID NO: 10)
CAGGTGCAACTAACACAGTGGGGCGCAGGACTGTTGAAGCCTTCGGAG
ACCCTGTCCCTCACCTGCGCTGTCGGTGGTGGGTCCTTCAGCGGTTAC
TTCTGGACTTGGATCCGCCAGTCCCCAGGGAGGGGCCTGGAGTGGATT
GGAGAAATCAATCACAAAGGAAAAACAACCTACAACCCGACCCTCAAG
AGTCGACTGAGCATATTGGTTGACACCTTCAAGAACCAATTTTCCCTC
AAATTGAGGTCTGTGGTCGCCGCGGACACGGCTGTCTATTACTGTGCG
AGAATTGTGGAGGCAGTGGGAGTTACGTCCTTTGACTCCTGGGGCCAG
GGAATCCTGGTCACCGTCTCGAGC
1242_P11 VH amino acid sequence
(SEQ ID NO: 9)
QVQLTQWGAGLLKPSETLSLTCAVGGGSFSGYFWTWIRQSPGRGLEWI
GEINHKGKTTYNPTLKSRLSILVDTFKNQFSLKLRSVVAADTAVYYCA
RIVEAVGVTSFDSWGQGILVTVSS
1242_P11 VL nucleotide sequence
(SEQ ID NO: 12)
CAGTCTGTATTGACGCAGCCGCCCTCAGTGTCTGCGGCCCCAGGACAG
AAGGTCACCATCTCCTGCTCTGGAAGCACCTCCAACATTGGAAATAAT
CATGTATCTTGGTACCAACAACTCCCACAGACAGCCCCCAAACTCCTC
ATTTATGACAATAATAAGCGACCCTCTGGGATTCCTGACCGATTCTCT
GGCTCCAAGTCTGGCGCGTCAGCCACCCTGGGCATCACCGGACTCCAG
ACTGGCGACGAGGCCTATTATTACTGCGGGACGTGGGATACCAGGTTG
AGTCGGGTGTTCGGCGGAGGAACCAAACTGACCGTTCTA
1242_P11 VL amino acid sequence
(SEQ ID NO: 11)
QSVLTQPPSVSAAPGQKVTISCSGSTSNIGNNHVSWYQQLPQTAPKLL
IYDNNKRPSGIPDRFSGSKSGASATLGITGLQTGDEAYYYCGTWDTRL
SRVFLGGGTKLTV

The 1253_N12 antibody (also referred to herein as N12) includes a heavy chain variable region (SEQ ID NO: 13) encoded by the nucleic acid sequence shown below in SEQ ID NO: 14, and a light chain variable region (SEQ ID NO: 15) encoded by the nucleic acid sequence shown in SEQ ID NO: 16.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{1h} edit., NIH Publication no. 91-3242 U.S. Department of Health and Human Services.) are highlighted in bold in the sequences below.

The heavy chain CDRs of the N12 antibody have the following sequences per Kabat definition: CDR1, GYFWT (SEQ ID NO: 57), CDR2, EINHRGSSSYNPSLRS (SEQ ID NO: 71) and CDR3, ITEAVGVTSFDS (SEQ ID NO: 72). The light chain CDRs of the N12 antibody have the following sequences per Kabat definition: CDR1, SGSTSNIGNNYVS (SEQ ID NO: 117), CDR2, DDDKRPS (SEQ ID NO: 73) and CDR3, GTWDSSLSRV (SEQ ID NO: 52).

The heavy chain CDRs of the N12 antibody have the following sequences per Chothia definition: CDR1, GGSFSG (SEQ ID NO: 55), CDR2, EINHRGSSS (SEQ ID NO: 74) and CDR3, ITEAVGVTSFDS (SEQ ID NO: 72). The light chain CDRs of the N12 antibody have the following sequences per Chothia definition: CDR1, SGSTSNIGNNYVS (SEQ ID NO: 117), CDR2, DDDKRPS (SEQ ID NO: 73) and CDR3, GTWDSSLSRV (SEQ ID NO: 52).

1253_N12 VH nucleotide sequence
(SEQ ID NO: 14)
CAGGTGCAGCTACAACAGTGGGGCGCAGGACTGGTGAAGCCTTCGGAG
ACCCTGTCCGTCACCTGCGCTGTCTATGGTGGGTCCTTCAGTGGTTAT
TTCTGGACTTGGATCCGCCAGCCCCCAGGGAGGGGACTGGAGTGGATT
GGAGAAATCAATCACAGAGGAAGTTCCTCCTACAACCCGTCTCTCAGG
AGTCGACTAAGTATATCAGTGGACACGTCCAAGAACCAGTTTTCCCTA
AAGATGAGGTCTGTGACCGCCGCGGACACGGCTGTTTATTACTGTGCG
AGGATTACGGAGGCGGTGGGAGTGACCTCCTTTGACTCCTGGGGCCAG
GGAGTCCTGGTCACCGTCTCGAGC
1253_N12 VH amino acid sequence
(SEQ ID NO: 13)
QVQLQQWGAGLVKPSETLSVTCAVYGGSFSGYFWTWIRQPPGRGLEWI
GEINHRGSSSYNPSLRSRLSISVDTSKNQFSLKMRSVTAADTAVYYCA
RITEAVGVTSFDSWGQGVLVTVSS
1253_N12 VL nucleotide sequence
(SEQ ID NO: 16)
CAGTCTGTATTGACGCAGCCGCCCTCAGTGTCTGCGGCCCCAGGACAA
AAGGTCACCATCTCCTGCTCTGGAAGCACCTCCAACATTGGAAATAAT
TATGTATCCTGGTACCAGCAACTCCCACAAAGAGCCCCCAAACTCCTC
ATTTATGACGATGATAAGCGACCCTCAGGGATTCCTGACCGATTCTCT
GGCTCCAAGTCTGGCGCGTCGGCCACCCTGGCCATCACCGGACTCCAG
ACTGGGGACGAGGCCTATTATTATTGCGGAACATGGGATAGTAGCCTG
AGTCGGGTGTTCGGCGGAGGGACCAAACTGACCGTTCTA
(SEQ ID NO: 15)
1253_N12 VL amino acid sequence
QSVLTQPPSVSAAPGQKVTISCSGSTSNIGNNYVSWYQQLPQRAPKLL
IYDDDKRPSGIPDRFSGSKSGASATLAITGLQTGDEAYYYCGTWDSS
LSRVFGGGTKLTVL

The 1256_B2 antibody (also referred to herein as B2) includes a heavy chain variable region (SEQ ID NO: 17) encoded by the nucleic acid sequence shown below in SEQ ID NO: 18, and a light chain variable region (SEQ ID NO: 19) encoded by the nucleic acid sequence shown in SEQ ID NO: 20.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the B2 antibody have the following sequences per Kabat definition: CDR1, GYFWS (SEQ ID NO: 75), CDR2, EINHRGSSTYKSSLKT (SEQ ID NO: 121) and CDR3, ITEAVGVTSFDS (SEQ ID NO: 72). The light chain CDRs of the B2 antibody have the following sequences per Kabat definition: CDR1, SGSTSNIGNNYVS (SEQ ID NO: 117), CDR2, DNDKRPS (SEQ ID NO: 61) and CDR3, GTWDNNLSRV (SEQ ID NO: 77).

The heavy chain CDRs of the B2 antibody have the following sequences per Chothia definition: CDR1, GGSFSG (SEQ ID NO: 55), CDR2, EINHRGSST (SEQ ID NO: 78) and CDR3, ITEAVGVTSFDS (SEQ ID NO: 72). The light chain CDRs of the B2 antibody have the following sequences per Chothia definition: CDR1, SGSTSNIGNNYVS (SEQ ID NO: 117), CDR2, DNDKRPS (SEQ ID NO: 61) and CDR3, GTWDNNLSRV (SEQ ID NO: 77).

1256_B2 VH nucleotide sequence
(SEQ ID NO: 18)
CAGGTGCAGCTACAACAGTGGGGCGCAGGACTGTTGAAGCCTTCGGAG
ACCCTGTCCCTCACCTGCGCTGTCTATGGTGGGTCCTTCAGTGGTTAT
TTCTGGAGTTGGATCCGCCAGCCCCCAGGGAGGGGACTGGAATGGATT
GGGGAAATCAATCACAGAGGAAGTTCCACCTACAAGTCGTCTCTCAAG
ACTCGACTAACCATGTCAGTAGACACGTCCAAGAACCAGTTTTCCATA
AAGCTGACTTCTGTGACCGCCGCGGACACGGCTGTTTATTATTGTGCG
AGAATTACGGAGGCGGTGGGAGTTACCTCCTTTGACTCCTGGGGCCAG
GGAGTCCTGGTCACCGTCTCGAGC
1256_B2 VH amino acid sequence
(SEQ ID NO: 17)
QVQLQQWGAGLLKPSETLSLTCAVYGGSFSGYFWSWIRQPPGRGLEWI
GEINHRGSSTYKSSLKTRLTMSVDTSKNQFSIKLTSVTAADTAVYYCA
RITEAVGVTSFDSWGQGVLVTVSS
1256_B2 VL nucleotide sequence
(SEQ ID NO: 20)
CAGTCTGTATTGACGCAGCCGCCCTCAGTGTCTGCGGCCCCAGGACAA
AAGGTCACCATCTCCTGCTCTGGAAGCACCTCCAACATTGGAAATAAT
TATGTATCCTGGTACCAGCAACTCCCACAAAGAGCCCCCAGACTCCTC
ATTTATGACAATGATAAGCGACCCTCAGGGATTCCTGACCGATTCTCT
GGCTCCAAGTCTGGCGCGTCAGCCACCCTGGGCATCACCGGACTCCAG
ACTGGGGACGAGGCCTATTATTATTGCGGAACATGGGATAATAACCTG
AGTCGGGTGTTCGGCGGAGGGACCAAACTGACCGTTCTA
1256_B2 VL amino acid sequence
(SEQ ID NO: 19)
QSVLTQPPSVSAAPGQKVTISCSGSTSNIGNNYVSWYQQLPQRAPRLL
IYDNDKRPSGIPDRFSGSKSGASATLGITGLQTGDEAYYYCGTWDNNL
SRVFGGGTKLTVL

The 1250_I13 antibody (also referred to herein as 113) includes a heavy chain variable region (SEQ ID NO: 21) encoded by the nucleic acid sequence shown below in SEQ ID NO: 22, and a light chain variable region (SEQ ID NO: 23) encoded by the nucleic acid sequence shown in SEQ ID NO: 24.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the 113 antibody have the following sequences per Kabat definition: CDR1, GYFWT (SEQ ID NO: 57), CDR2, EINHRGTSS (SEQ ID NO: 79) and CDR3, ITEAVGFTSFDY (SEQ ID NO: 80). The light chain CDRs of the 113 antibody have the following sequences per Kabat definition: CDR1, SGSTSNIGSNYVS (SEQ ID NO: 118), CDR2, DNDKRPS (SEQ ID NO: 61) and CDR3, GTWDSSLSRV (SEQ ID NO: 52).

The heavy chain CDRs of the 113 antibody have the following sequences per Chothia definition: CDR1, GGSFSG (SEQ ID NO: 55), CDR2, EINHRGTSS (SEQ ID NO: 79) and CDR3, ITEAVGFTSFDY (SEQ ID NO: 80). The light chain CDRs of the 113 antibody have the following sequences per Chothia definition: CDR1, SGSTSNIGSNYVS (SEQ ID NO: 118), CDR2, DNDKRPS (SEQ ID NO: 61) and CDR3, GTWDSSLSRV (SEQ ID NO: 52).

1250_I13 VH nucleotide sequence
(SEQ ID NO: 22)
CAGGTGCAGCTACAACAGTGGGGCGCAGGACTGTTGAAGCCTTCGGAG
ACCCTGTCCCTCACCTGCGCTGTCTATGGTGGGTCCTTCAGTGGTTAT
TTCTGGACTTGGGTCCGCCAGCTCCCAGGGAGGGGACTGGAGTGGATT
GGAGAGATCAATCACAGAGGAACTTCCTCCTACAACCCGTCTCTCAGG
AGTCGACTAACCATATCAGTAGACACGTCCAAGAACCAGTTTTCCCTA
AAACTGAGTTCTGTGACCGCCGCGGACACGGCTGTTTATTATTGTGCG
AGAATTACGGAGGCGGTGGGCTTTACCTCCTTTGACTACTGGGGCCAG
GGAATCCTGGTCACCGTCTCGAGC
1250_I13 VH amino acid sequence
(SEQ ID NO: 21)
QVQLQQWGAGLLKPSETLSLTCAVYGGSFSGYFWTWVRQLPGRGLEWI
GEINHRGTSSYNPSLRSRLTISVDTSKNQFSLKLSSVTAADTAVYYCA
RITEAVGFTSFDYWGQGILVTVSS
1250_I13 VL nucleotide sequence
(SEQ ID NO: 24)
CAGTCTGTATTGACGCAGCCGCCCTCAATGTCTGCGGCCCCAGGACAA
AAGGTCACCATCTCCTGCTCTGGAAGCACCTCCAACATTGGAAGTAAT
TATGTATCCTGGTACCAGCAACTCCCACAAAGAGCCCCCAAACTCCTC
ATTTATGACAATGATAAGCGACCCTCAGGGATTCCTGACCGATTCTCT
GGCTCCAAGTCTGGCGCGTCAGCCACCCTGGACATCACCGGACTCCAG
ACTGGGGACGAGGCCTATTATTATTGCGGAACATGGGATAGTAGCCTG
AGTCGGGTGTTCGGCGGAGGGACCAAACTGACCGTTCTA
1250_I13 VL amino acid sequence
(SEQ ID NO: 23)
QSVLTQPPSNISAAPGQKVTISCSGSTSNIGSNYVSWYQQLPQRAPKL
LIYDNDKRPSGIPDRFSGSKSGASATLDITGLQTGDEAYYYCGTWDSS
LSRVFGGGTKLTVL

The 1252_B7 antibody (also referred to herein as B7) includes a heavy chain variable region (SEQ ID NO: 25) encoded by the nucleic acid sequence shown below in SEQ ID NO: 26, and a light chain variable region (SEQ ID NO: 27) encoded by the nucleic acid sequence shown in SEQ ID NO: 28.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the B7 antibody have the following sequences per Kabat definition: CDR1, GYFWS (SEQ ID NO: 75), CDR2, EINHSGSTNYNPSLKS (SEQ ID NO: 50) and CDR3, GMVVAGTRSDAFDI (SEQ ID NO: 64). The light chain CDRs of the B7 antibody have the following sequences per Kabat definition: CDR1, SGSSSNIGINTVN (SEQ ID NO: 81), CDR2, SNNQRPS (SEQ ID NO: 76) and CDR3, AAWDDSLNEV (SEQ ID NO: 85).

The heavy chain CDRs of the B7 antibody have the following sequences per Chothia definition: CDR1, IGSFRG (SEQ ID NO: 86), CDR2, EINHSGSTN (SEQ ID NO: 56) and CDR3, GMVVAGTRSDAFDI (SEQ ID NO: 64). The light chain CDRs of the B7 antibody have the following sequences per Chothia definition: CDR1, SGSSSNIGINTVN (SEQ ID NO: 81), CDR2, SNNQRPS (SEQ ID NO: 76) and CDR3, AAWDDSLNEV (SEQ ID NO: 85).

1252_B7 VH nucleotide sequence
(SEQ ID NO: 26)
CAGGTGCAGCTACAGCAGTGGGGCACAGGGCTGTTGAAGCCTTCGGAG
ACCCTGTCCCGCACCTGCGCTGTCTATATTGGGTCCTTCCGTGGTTAC
TTCTGGAGCTGGCTCCGCCAGCCCCCAGGGAAGGGGCTGGAGTGGATT
GGGGAGATCAATCATAGTGGAAGCACCAACTACAACCCGTCCCTCAAG
AGTCGAGTCACCATATCAGTAGACACGTCCAAGAACCAGTTCTCCCTG
AAGCTGACCTCTGTGACCGCCGCGGACACGGCTGTGTATTACTGTGCG
AGAGGCATGGTAGTGGCTGGAACCCGGAGTGATGCTTTTGATATCTGG
GGCCAAGGGACACTGGTCACCGTCTCCTCA
1252_B7 VH amino acid sequence
(SEQ ID NO: 25)
QVQLQQWGTGLLKPSETLSRTCAVYIGSFRGYFWSWLRQPPGKGLEWI
GEINHSGSTNYNPSLKSRVTISVDTSKNQFSLKLTSVTAADTAVYYCA
WRGMVVAGTRSDAFDIGQGTLVTVSS
1252_B7 VL nucleotide sequence
(SEQ ID NO: 28)
CAGAGTGTGCTGACTCAGCCACCCTCAGCGTCTGGGACCCCCGGGCAG
AGGGTCACCATCTCTTGTTCTGGAAGCAGCTCCAACATCGGAATTAAT
ACTGTAAACTGGTACCAACAACTCCCAGGAACGGCCCCCAGACTCGTC
ATCTATAGCAATAATCAGCGGCCCTCAGGGGTCCCTGCCCGATTTTCT
GGCTCCAAGTCTGGCACCTCAGCCTCCCTGGCCATCAATGGGCTCCAG
GCTGAGGATGAGGCTGATTATTACTGTGCAGCATGGGATGACAGCCTG
AATGAAGTATTCGGCGGAGGGACCAAGCTGACCGTTCTA
1252_B7 VL amino acid sequence
(SEQ ID NO: 27)
QSVLTQPPSASGTPGQRVTISCSGSSSNIGINTVNWYQQLPGTAPRLV
IYSNNQRPSGVPARFSGSKSGTSASLAINGLQAEDEADYYCAAWDDSL
NEVFGGGTKLTVL

The 1248_C17 antibody (also referred to herein as C17) includes a heavy chain variable region (SEQ ID NO: 29) encoded by the nucleic acid sequence shown below in SEQ ID NO: 30, and a light chain variable region (SEQ ID NO: 31) encoded by the nucleic acid sequence shown in SEQ ID NO: 32.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the C17 antibody have the following sequences per Kabat definition: CDR1, GYFWS (SEQ ID NO: 75), CDR2, EINHSGSTNYNPSLKS (SEQ ID NO: 50) and CDR3, GIVVAGTRSDAFDI (SEQ ID NO: 82). The light chain CDRs of the C17 antibody have the following sequences per Kabat definition: CDR1, SGSSSNIGINTVN (SEQ ID NO: 81), CDR2, NNNQRPS (SEQ ID NO: 83) and CDR3, AAWDDSLNEV (SEQ ID NO: 85).

The heavy chain CDRs of the C17 antibody have the following sequences per Chothia definition: CDR1, IGSFRG (SEQ ID NO: 86), CDR2, EINHSGSTN (SEQ ID NO: 56) and CDR3, GIVVAGTRSDAFDI (SEQ ID NO: 82). The light chain CDRs of the C17 antibody have the following sequences per Chothia definition: CDR1, SGSSSNIGINTVN (SEQ ID NO: 81), CDR2, NNNQRPS (SEQ ID NO: 83) and CDR3, AAWDDSLNEV (SEQ ID NO: 85).

1248_C17 VH nucleotide sequence
(SEQ ID NO: 30)
CAGGTGCAGCTACAGCAGTGGGGCGCAGGGCTGTTGAAGCCTTCGGAG
ACCCTGTCCCGCACCTGCGCTGTCTATATTGGGTCCTTCCGTGGTTAC
TTCTGGAGCTGGCTCCGCCAGCCCCCAGGGAAGGGGCTGGAGTGGATT
GGGGAGATCAATCATAGTGGAAGCACCAACTACAACCCGTCCCTCAAG
AGTCGAGTCACCATGTCAGTAGACACGTCCAAGAACCAGTTCTCCCTG
AAGCTGACCTCTGTGACCGCCGCGGACACGGCTGTGTATTACTGTGCG
AGAGGCATAGTCGTGGCTGGTACCCGGAGTGATGCTTTTGATATTTGG
GGCCAAGGGACACTGGTCACCGTCTCGAGC
1248_C17 VH amino acid sequence
(SEQ ID NO: 29)
QVQLQQWGAGLLKPSETLSRTCAVYIGSFRGYFWSWLRQPPGKGLEWI
GEINHSGSTNYNPSLKSRVTMSVDTSKNQFSLKLTSVTAADTAVYYCA
RGIVVAGTRSDAFDIWGQGTLVTVSS
1248_C17 VL nucleotide sequence
(SEQ ID NO: 32)
CAGTCTGTGCTGACTCAGCCACCCTCAGCGTCTGGGACCCCCGGGCAG
AGGGTCACCATCTCTTGTTCTGGAAGCAGCTCCAACATCGGAATAAAC
ACTGTAAATTGGTACCAACAACTCCCAGGAACGGCCCCCAGACTCGTC
ATCTATAACAATAATCAGCGGCCCTCAGGGGTCCCTGCCCGATTCTCT
GGCTCCAAGTCTGGCACCTCAGCCTCCCTGGCCATCAATGGGCTCCAG
TCTGAGGATGAGGCTGATTATTACTGTGCAGCATGGGATGACAGTCTG
AATGAAGTATTCGGCGGAGGGACCAAGCTGACCGTTCTA
1248_C17 VL amino acid sequence
(SEQ ID NO: 31)
QSVLTQPPSASGTPGQRVTISCSGSSSNIGINTVNWYQQLPGTAPRLV
IYNNNQRPSGVPARFSGSKSGTSASLAINGLQSEDEADYYCAAWDDSL
NEVFGGGTKLTVL

The 1247_A18 antibody (also referred to herein as A18) includes a heavy chain variable region (SEQ ID NO: 33) encoded by the nucleic acid sequence shown below in SEQ ID NO: 34, and a light chain variable region (SEQ ID NO: 35) encoded by the nucleic acid sequence shown in SEQ ID NO: 36.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the A18 antibody have the following sequences per Kabat definition: CDR1, SYWMN (SEQ ID NO: 87), CDR2, NINQDGTEKNYVDSVKG (SEQ ID NO: 88) and CDR3, GVFQGAPHFVF (SEQ ID NO: 89). The light chain CDRs of the A18 antibody have the following sequences per Kabat definition: CDR1, RSSQSLLHGNGFNYLD (SEQ ID NO: 90), CDR2, LGSDRAS (SEQ ID NO: 91) and CDR3, MQSLRTPLT (SEQ ID NO: 92).

The heavy chain CDRs of the A18 antibody have the following sequences per Chothia definition: CDR1, (SEQ ID NO: 93), CDR2, NINQDGTEKN (SEQ ID NO: 94) and CDR3, GVFQGAPHFVF (SEQ ID NO: 89). The light chain CDRs of the A18 antibody have the following sequences per Chothia definition: CDR1, RSSQSLLHGNGFNYLD (SEQ ID NO: 90), CDR2, LGSDRAS (SEQ ID NO: 91) and CDR3, MQSLRTPLT (SEQ ID NO: 92).

1247_A18 VH nucleotide sequence
(SEQ ID NO: 34)
GAGGTGCGCCTGGTGCAGTCTGGGGGAGGCTTGGTCCAGCCTGGGAGG
TCCCTGAGACTCTCCTGTGCAGCCTTTGAATTCACCTTTGGGAGTTAT
TGGATGAACTGGGTCCGCCAGGCTCCAGGGAAGGGGCTGGAGTGGGTG
GCCAACATAAACCAAGATGGAACTGAGAAAAACTATGTGGACTCTGTG
AAGGGCCGATTCACCATCTCCAGAGACAACACCAAGAATTCACTGTAT
CTGGAAATGGACAACTTGAGAGCCGACGACACGGGTATTTATTACTGT
GCGAGAGGTGTTTTCCAGGGGGCCCCACATTTTGTCTTCTGGGGCCAG
GGAGCCCTGGTCACCGTCTCGAGC
1247_A18 VH amino acid sequence
(SEQ ID NO: 33)
EVRLVQSGGGLVQPGRSLRLSCAAFEFTFGSYWMNWVRQAPGKGLEWV
ANINQDGTEKNYVDSVKGRFTISRDNTKNSLYLEMDNLRADDTGIYYC
ARGVFQGAPHFVFWGQGALVTVSS
1247_A18 VL nucleotide sequence
(SEQ ID NO: 36)
GAGACTGTACTTACTCAGTCTCCACTTTCCCTGGCCGTCACCCCTGGA
GAGCCGGCCTCCATCTCCTGCAGGTCTAGTCAGAGCCTCCTACATGGT
AATGGATTCAACTATTTGGATTGGTACCTGCAGAAGCCAGGCCAGTCT
CCACAGCTCCTGATCTACTTGGGTTCTGATCGGGCCTCCGGGGTCCCT
GACAGGTTCAGTGGCAGTGGATCAGGCACAGATTTTACACTGAAAATT
AGCAGGGTGGAGACTGAGGATGTTGGCGTTTATTACTGCATGCAAAGT
CTACGCACTCCTCTAACTTTTGGCCAGGGGACCAAACTGGAGATCAAA
1247_A18 VL amino acid sequence
(SEQ ID NO: 35)
ETVLTQSPLSLAVTPGEPASISCRSSQSLLHGNGFNYLDWYLQKPGQS
PQLLIYLGSDRASGVPDRFSGSGSGTDFTLKISRVETEDVGVYYCMQ
SLRTPLTFGQGTKLEIK

The 1252_O13 antibody (also referred to herein as 013) includes a heavy chain variable region (SEQ ID NO: 37) encoded by the nucleic acid sequence shown below in SEQ ID NO: 38, and a light chain variable region (SEQ ID NO:39) encoded by the nucleic acid sequence shown in SEQ ID NO:40.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the 013 antibody have the following sequences per Kabat definition: CDR1, RYDIS (SEQ ID NO: 95), CDR2, WMNPNSGNTGYAQKFQD (SEQ ID NO: 96) and CDR3, LRVESLGRRFFYAYNGMDV (SEQ ID NO: 97). The light chain CDRs of the 013 antibody have the following sequences per Kabat definition: CDR1, QASQDISNYLN (SEQ ID NO: 98), CDR2, DASNLET (SEQ ID NO: 99) and CDR3, QQYNNVLFT (SEQ ID NO: 100).

The heavy chain CDRs of the 013 antibody have the following sequences per Chothia definition: CDR1, GYTFNR (SEQ ID NO: 101), CDR2, WMNPNSGNTG (SEQ ID NO: 102) and CDR3, LRVESLGRRFFYAYNGMDV (SEQ ID NO: 97). The light chain CDRs of the 013 antibody have the following sequences per Chothia definition: CDR1, QASQDISNYLN (SEQ ID NO: 98), CDR2, DASNLET (SEQ ID NO: 99) and CDR3, QQYNNVLFT (SEQ ID NO: 100).

1252_O13 VH nucleotide sequence
(SEQ ID NO: 38)
CAGGTGCAGCTGGTGCAGTCTGGGGCTGAGGTGAAGAAGCCTGGGGCC
TCAGTGAAGGTCTCCTGCAAGGCTTCTGGCTACACCTTCAATAGGTAT
GATATCAGCTGGGTGCGACAGGCCACTGGACAAGGGCTTGAGTGGATG
GGATGGATGAATCCTAACAGTGGGAACACAGGGTATGCACAGAAGTTC
CAGGACAGAGTCACCATGACCAGGAACACCTCCATAAGAACAGCCTAC
ATGGAGCTGAGCAGTCTGAGATCTGACGACACGGCCGTGTATTACTGT
GCGAGTCTTAGGGTGGAATCGCTGGGCCGTCGTTTTTTCTACGCCTAC
AATGGTATGGACGTCTGGGGCCAAGGGACCACGGTCACCGTCTCGAGC
1252_O13 VH amino acid sequence
(SEQ ID NO: 37)
QVQLVQSGAEVKKPGASVKVSCKASGYTFNRYDISWVRQATGQGLEWM
GWMNPNSGNTGYAQKFQDRVTMTRNTSIRTAYMELSSLRSDDTAVYYC
ASLRVESLGRRFFYAYNGMDVWGQGTTVTVSS
1252_O13 VL nucleotide sequence
(SEQ ID NO: 40)
GACATCCAGATGACCCAGTCTCCATCCTCCCTGTCTGCATCTGTAGGA
GACAGAGTCACCATCTCTTGCCAGGCGAGTCAGGACATTAGCAACTAT
TTAAATTGGTATCAGCAGAAACCAGGGAAAGCCCCTAAACTCCTGATC
TACGATGCATCCAATTTGGAAACAGGGGTCCCATCAAGGTTCAGTGGA
AGTGGATCTGGGACAGATTTTACTTTCACTATCAGCAGCCTGCAGCCT
GAAGATATTGCAACATATTACTGTCAACAATATAATAATGTCCTTTTC
ACTTTCGGCGGGGGGACCAAGGTGGAGATCAAA
1252_O13 VL amino acid sequence
(SEQ ID NO: 39)
DIQMTQSPSSLSASVGDRVTISCQASQDISNYLNWYQQKPGKAPKLLI
YDASNLETGVPSRFSGSGSGTDFTFTISSLQPEDIATYYCQQYNNVLF
TFGGGTKVEIK

The 1038_D5 (TCN-445) antibody (also referred to herein as D5) includes a heavy chain variable region (SEQ ID NO: 41) encoded by the nucleic acid sequence shown below in SEQ ID NO: 42, and a light chain variable region (SEQ ID NO: 43) encoded by the nucleic acid sequence shown in SEQ ID NO: 44.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the D5 antibody have the following sequences per Kabat definition: CDR1, DFYFH (SEQ ID NO: 103), CDR2, WINPRSGATNYAHKFRG (SEQ ID NO: 104) and CDR3, DMRRENGYNFDGTFDY (SEQ ID NO: 105). The light chain CDRs of the D5 antibody have the following sequences per Kabat definition: CDR1, QASQDIKNYLN (SEQ ID NO: 106), CDR2, DASNLET (SEQ ID NO: 99) and CDR3, QRYDAFPLT (SEQ ID NO: 107).

The heavy chain CDRs of the D5 antibody have the following sequences per Chothia definition: CDR1, GYTFTD (SEQ ID NO: 108), CDR2, WINPRSGATN (SEQ ID NO: 109) and CDR3, DMRRENGYNFDGTFDY (SEQ ID NO: 105). The light chain CDRs of the D5 antibody have the following sequences per Chothia definition: CDR1, QASQDIKNYLN (SEQ ID NO: 106), CDR2, DASNLET (SEQ ID NO: 99) and CDR3, QRYDAFPLT (SEQ ID NO: 107).

1038_D5 (TCN-445) VH nucleotide sequence
(SEQ ID NO: 42)
CAGGTGCAATTGGTGCAGTCTGGGGCTGAGGTACAGAAGCCTGGGGCC
TCAATGAAAGTCTCCTGCAAGGCTTCTGGATACACCTTCACCGACTTC
TATTTTCATTGGGTGCGACAGGCCCCTGGACAAGGCCTTGAGTGGATG
GGATGGATCAACCCTAGAAGTGGTGCCACGAATTATGCACATAAATTT
CGGGGCAGGGTCACTATGACCAGTGACACGTCCATCAAAACAATCTAC
ATGAGTCTTGGTTGGCTGAGATCTGGTGACACGGCCGTATATTACTGT
GCGCGGGATATGAGACGTGAAAACGGTTACAATTTTGATGGGACTTTC
GACTACTGGGGCCAGGGAACCCTAGTCACCGTCTCGAGC
1038_D5 (TCN-445) VH amino acid sequence
(SEQ ID NO: 41)
QVQLVQSGAEVQKPGASMKVSCKASGYTFTDFYFHWVRQAPGQGLEWM
GWINPRSGATNYAHKFRGRVTMTSDTSIKTIYMSLGWLRSGDTAVYYC
ARDMRRENGYNFDGTFDYWGQGTLVTVSS
1038_D5 (TCN-445) VL nucleotide sequence
(SEQ ID NO: 44)
GACATCCAGATGACCCAGTCTCCACCCTCCCTGTCTGCATCTGTTGGA
GACAGAGTCACCATCACTTGCCAGGCGAGTCAGGACATTAAGAACTAT
TTAAATTGGTATCAGCAAAAACCAGGGAAAGCCCCTAAACTCCTGATC
TACGATGCATCCAATTTGGAAACAGGGGTCCCGTCAAGGTTCAGTGGA
AGTAGATCTGGGACAAATTTTACTTTCACCATCAACAGCCTGCAGCCT
GAGGATATTGCAACATATTTCTGTCAACGGTATGACGCTTTCCCGCTC
ACTTTCGGCGGTGGGTCCAAGGTGGAGATCAAA
1038_D5 (TCN-445) VL amino acid sequence
(SEQ ID NO: 43)
DIQMTQSPPSLSASVGDRVTITCQASQDIKNYLNWYQQKPGKAPKLLI
YDASNLETGVPSRFSGSRSGTNFTFTINSLQPEDIATYFCQRYDAFPL
TFGGGSKVEIK

The 1261_P5 antibody (also referred to herein as P5) includes a heavy chain variable region (SEQ ID NO: 45) encoded by the nucleic acid sequence shown below in SEQ ID NO: 46 and a light chain variable region (SEQ ID NO: 47) encoded by the nucleic acid sequence shown in SEQ ID NO: 48.

The amino acids encompassing the CDRs as defined by Chothia, C. et al. (1989, Nature, 342: 877-883) are underlined and those defined by Kabat E. A. et al. (1991, Sequences of Proteins of Immunological Interest, 5^{th} edit., NIH Publication no. 91-3242 U.S. Department of Health and Human Services.) are highlighted in bold in the sequences below.

The heavy chain CDRs of the P5 antibody have the following sequences per Kabat definition: CDR1, SYWMS (SEQ ID NO: 119), CDR2, NIKQDGSEKYYVDSVKG (SEQ ID NO: 110) and CDR3, DSEVAAAGTHFHY (SEQ ID NO: 111). The light chain CDRs of the P5 antibody have the following sequences per Kabat definition: CDR1, RASQSISTYLN (SEQ ID NO: 112), CDR2, AASSLQS (SEQ ID NO: 113) and CDR3, QQSYTALT (SEQ ID NO: 114).

The heavy chain CDRs of the P5 antibody have the following sequences per Chothia definition: CDR1, GFSFSS (SEQ ID NO: 84), CDR2, NIKQDGSEKY (SEQ ID NO: 120) and CDR3, DSEVAAAGTHFHY (SEQ ID NO: 111). The light chain CDRs of the P5 antibody have the following sequences per Chothia definition: CDR1, RASQSISTYLN (SEQ ID NO: 112), CDR2, AASSLQS (SEQ ID NO: 113) and CDR3, QQSYTALT (SEQ ID NO: 114).

1261_P5 VH nucleotide sequence
(SEQ ID NO: 46)
GAGGTGCAGCTGGTGGAGTCTGGGGGAGGCTTGGTCCAGCCTGGGGGG
TCCCTGAGACTCTCCTGTGCAGCCTCTGGATTCAGCTTTAGTAGTTAT
TGGATGAGCTGGGTCCGCCAGGCTCCAGGGAAGGGGCTGGAGTGGGTG
GCCAACATAAAGCAAGATGGAAGTGAGAAATACTATGTGGACTCTGTG
AAGGGCCGATTCACCATCTCCAGAGACAACGCCAAGAACTCACTGTAT
CTGCAAATGAACAGCCTGAGAGCCGAGGACACGGCCGTATATTACTGT
GCGAGAGATTCCGAAGTGGCAGCAGCTGGTACACACTTTCACTACTGG
GGCCAGGGAACCCTGGTCACCGTCTCCTCA
1261_P5 VH amino acid sequence
(SEQ ID NO: 45)
EVQLVESGGGLVQPGGSLRLSCAASGFSFSSYWMSWVRQAPGKGLEWV
ANIKQDGSEKYYVDSVKGRFTISRDNAKNSLYLQMNSLRAEDTAVYYC
ARDSEVAAAGTHFHYWGQGTLVTVSS
1261_P5 VL nucleotide sequence
(SEQ ID NO: 48)
GACATCCAGATGACCCAGTCTCCATCCTCCCTGTCTACATCTGTAGGA
GACAGAGTCACCATCACTTGCCGGGCAAGTCAGAGCATTAGCACTTAT
TTAAATTGGTATCAACAGAAACCAGGGAAAGCCCCTAACCTCCTGATC
TATGCTGCATCCAGTTTGCAAAGTGGGGTCCCATCAAGGTTCAGTGGC
AGTGGATCTGGGACAGATTTCACTCTCACCATCAGCAGTCTGCAACCT
GAAGATTTTGCAACTTACTACTGTCAACAGAGTTACACTGCGCTCACT
TTCGGCGGAGGGACCAAGGTGGAGATCAAA
1261_P5 VL amino acid sequence
(SEQ ID NO: 47)
DIQMTQSPSSLSTSVGDRVTITCRASQSISTYLNWYQQKPGKAPNLLI
YAASSLQSGVPSRFSGSGSGTDFTLTISSLQPEDFATYYCQQSYTALT
FGGGTKVEIK

HuCA antibodies of the invention also include antibodies that include a heavy chain variable region amino acid sequence that is at least 90%, 92%, 95%, 97% 98%, 99% or more identical the amino acid sequence of SEQ ID NO: 1, 5, 9, 13, 17, 21, 25, 29, 33, 37, 41, or 45 and/or a light chain variable region amino acid that is at least 90%, 92%, 95%, 97% 98%, 99% or more identical the amino acid sequence of SEQ ID NO: 3, 7, 11, 15, 19, 23, 27, 31, 35, 39, 43, or 47.

Alternatively, the monoclonal antibody is an antibody that binds to the same epitope as 1061_I16 (TCN-462), 1226_K16, 1242_P11, 1242_N12, 1256_B2, 1250_I13, 1252_B7, 1248_C17, 1247_A18, 1252_O13, 1038_D5 (TCN-445), and 1261_P5. Moreover, the monoclonal antibody is an antibody that binds to the same epitope as a huCA antibody described herein.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following definitions are useful in understanding the present invention:

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α)), delta (δ)), epsilon (ε), gamma (γ and mu (μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2. An IgM antibody consists of 5 of the basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}).

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). The present invention provides variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H}1, C_{H} 2 and C_{H} 3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an cancer cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the cancer cell.

As used herein, an antibody is said to be “immunospecific,” “specific for” or to “specifically bind” an antigen if it reacts at a detectable level with the antigen, preferably with an affinity constant, Ka, of greater than or equal to about 104 M-1, or greater than or equal to about 105 M-1, greater than or equal to about 106 M-1, greater than or equal to about 107 M-1, or greater than or equal to 108 M⁻¹. Affinity of an antibody for its cognate antigen is also commonly expressed as a dissociation constant K_{D}, and in certain embodiments, HuCA antibody specifically binds to a cancer antigen if it binds with a K_{D} of less than or equal to 10⁻4 M, less than or equal to about 10⁻5 M, less than or equal to about 10⁻6 M, less than or equal to 10⁻7 M, or less than or equal to 10⁻8 M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist” antibody is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

An antibody that “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). Preferably the cell is a cancer cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody that induces apoptosis is one that results in about 2 to 50 fold, preferably about 5 to 50 fold, and most preferably about 10 to 50 fold, induction of annexin binding relative to untreated cell in an annexin binding assay.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., PNAS (USA) 95:652-656 (1998).

“Fc receptor” or “FcR” describes a receptor that binds to the Fc region of an antibody. In certain embodiments, the FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FCγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes that express one or more FcRs and perform effector functions. Preferably, the cells express at least FcγRIII and perform ADCC effector function. Examples of human leukocytes that mediate ADCC include PBMC, NK cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, e.g., as described in Gazzano-Santoro et al., J. Immunol. Methods 202:163 (1996), may be performed.

A “mammal” for purposes of treating n infection, refers to any mammal, including humans, domestic and farm animals, and zoo, sports, or pet animals, such as dogs, cats, cattle, horses, sheep, pigs, goats, rabbits, etc. Preferably, the mammal is human.

“Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for an infection if, after receiving a therapeutic amount of an antibody according to the methods of the present invention, the patient shows observable and/or measurable reduction in or absence of one or more of the following: reduction in the number of tumor cells or absence of the tumor cells, one or more of the symptoms associated with the cancer; reduced morbidity and mortality, and improvement in quality of life issues. The above parameters for assessing successful treatment and improvement in the disease are readily measurable by routine procedures familiar to a physician.

The term “therapeutically effective amount” refers to an amount of an antibody or a drug effective to “treat” a disease or disorder in a subject or mammal. See preceding definition of “treating.”

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Administration “in combination with” one or more further therapeutic agents includes simultaneous (concurrent) and consecutive administration in any order.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below.

A “growth inhibitory agent” when used herein refers to a compound or composition which inhibits growth of a cell, either in vitro or in vivo. Examples of growth inhibitory agents include agents that block cell cycle progression, such as agents that induce G1 arrest and M-phase arrest. Classical M-phase blockers include the vinca alkaloids (vincristine, vinorelbine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE™, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Label” as used herein refers to a detectable compound or composition that is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition that is detectable.

The term “epitope tagged” as used herein refers to a chimeric polypeptide comprising a polypeptide fused to a “tag polypeptide.” The tag polypeptide has enough residues to provide an epitope against which an antibody can be made, yet is short enough such that it does not interfere with activity of the polypeptide to which it is fused. The tag polypeptide is also preferably fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

A “small molecule” is defined herein to have a molecular weight below about 500 Daltons.

The terms “nucleic acid” and “polynucleotide” are used interchangeably herein to refer to single- or double-stranded RNA, DNA, or mixed polymers. Polynucleotides may include genomic sequences, extra-genomic and plasmid sequences, and smaller engineered gene segments that express, or may be adapted to express polypeptides.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising CDRs and being capable of binding a cancer antigen or cancer cell.

An “isolated polypeptide” is one that has been identified and separated and/or recovered from a component of its natural environment. In preferred embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature or can be produced by recombinant or synthetic means.

A polynucleotide “variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of the invention and evaluating one or more biological activities of the encoded polypeptide as described herein and/or using any of a number of techniques well known in the art.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating one or more biological activities of the polypeptide as described herein and/or using any of a number of techniques well known in the art.

Modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences that encode said peptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

“Homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least 70%, at least 75%, at least 80%, at least 90%, at least 95%, at least 98%, or at least 99% polynucleotide or polypeptide homology with a polynucleotide or polypeptide described herein.

“Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells. Suitable vectors are disclosed below.

As used in this specification and the appended claims, the singular forms “a,” “an” and “the” include plural references unless the content clearly dictates otherwise.

As will be understood by the skilled artisan, general description of antibodies herein and methods of preparing and using the same also apply to individual antibody polypeptide constituents and antibody fragments.

The antibodies of the present invention may be polyclonal or monoclonal antibodies. However, in preferred embodiments, they are monoclonal. In particular embodiments, antibodies of the present invention are fully human antibodies. Methods of producing polyclonal and monoclonal antibodies are known in the art and described generally, e.g., in U.S. Pat. No. 6,824,780. Typically, the antibodies of the present invention are produced recombinantly, using vectors and methods available in the art, as described further below. Human antibodies may also be generated by in vitro activated B cells (see U.S. Pat. Nos. 5,567,610 and 5,229,275).

Human antibodies may also be produced in transgenic animals (e.g., mice) that are capable of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice results in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WO 97/17852. Such animals may be genetically engineered to produce human antibodies comprising a polypeptide of the present invention.

In certain embodiments, antibodies of the present invention are chimeric antibodies that comprise sequences derived from both human and non-human sources. In particular embodiments, these chimeric antibodies are humanized or Primatized™. In practice, humanized antibodies are typically human antibodies in which some hypervariable region residues and possibly some FR residues are substituted by residues from analogous sites in rodent antibodies.

In the context of the present invention, chimeric antibodies also include fully human antibodies wherein the human hypervariable region or one or more CDRs are retained, but one or more other regions of sequence have been replaced by corresponding sequences from a non-human animal.

The choice of non-human sequences, both light and heavy, to be used in making the chimeric antibodies is important to reduce antigenicity and human anti-non-human antibody responses when the antibody is intended for human therapeutic use. It is further important that chimeric antibodies retain high binding affinity for the antigen and other favorable biological properties. To achieve this goal, according to a preferred method, chimeric antibodies are prepared by a process of analysis of the parental sequences and various conceptual chimeric products using three-dimensional models of the parental human and non-human sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

As noted above, antibodies (or immunoglobulins) can be divided into five different classes, based on differences in the amino acid sequences in the constant region of the heavy chains. All immunoglobulins within a given class have very similar heavy chain constant regions. These differences can be detected by sequence studies or more commonly by serological means (i.e. by the use of antibodies directed to these differences). Antibodies, or fragments thereof, of the present invention may be any class, and may, therefore, have a gamma, mu, alpha, delta, or epsilon heavy chain. A gamma chain may be gamma 1, gamma 2, gamma 3, or gamma 4; and an alpha chain may be alpha 1 or alpha 2.

In a preferred embodiment, an antibody of the present invention, or fragment thereof, is an IgG. IgG is considered the most versatile immunoglobulin, because it is capable of carrying out all of the functions of immunoglobulin molecules. IgG is the major Ig in serum, and the only class of Ig that crosses the placenta. IgG also fixes complement, although the IgG4 subclass does not. Macrophages, monocytes, PMN's and some lymphocytes have Fc receptors for the Fc region of IgG. Not all subclasses bind equally well; IgG2 and IgG4 do not bind to Fc receptors. A consequence of binding to the Fc receptors on PMN's, monocytes and macrophages is that the cell can now internalize the antigen better. IgG is an opsonin that enhances phagocytosis. Binding of IgG to Fc receptors on other types of cells results in the activation of other functions. Antibodies of the present invention may be of any IgG subclass.

In another preferred embodiment, an antibody, or fragment thereof, of the present invention is an IgE. IgE is the least common serum Ig since it binds very tightly to Fc receptors on basophils and mast cells even before interacting with antigen. As a consequence of its binding to basophils and mast cells, IgE is involved in allergic reactions. Binding of the allergen to the IgE on the cells results in the release of various pharmacological mediators that result in allergic symptoms. IgE also plays a role in parasitic helminth diseases. Eosinophils have Fc receptors for IgE and binding of eosinophils to IgE-coated helminths results in killing of the parasite. IgE does not fix complement.

In various embodiments, antibodies of the present invention, and fragments thereof, comprise a variable light chain that is either kappa or lambda. The lambda chain may be any of subtype, including, e.g., lambda 1, lambda 2, lambda 3, and lambda 4.

As noted above, the present invention further provides antibody fragments comprising a polypeptide of the present invention. In certain circumstances there are advantages of using antibody fragments, rather than whole antibodies. For example, the smaller size of the fragments allows for rapid clearance, and may lead to improved access to certain tissues, such as solid tumors. Examples of antibody fragments include: Fab, Fab′, F(ab′)₂ and Fv fragments; diabodies; linear antibodies; single-chain antibodies; and multispecific antibodies formed from antibody fragments.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner.

In other embodiments, the antibody of choice is a single chain Fv fragment (scFv). See WO 93/16185; U.S. Pat. Nos. 5,571,894; and 5,587,458. Fv and sFv are the only species with intact combining sites that are devoid of constant regions. Thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

In certain embodiments, antibodies of the present invention are bispecific or multi-specific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-Cancer arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and FcγRIII (CD16), so as to focus and localize cellular defense mechanisms to the cancer cell. Bispecific antibodies may also be used to localize cytotoxic agents to cancer cells. These antibodies possess an Cancer-binding arm and an arm that binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies). WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H} 3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147: 60 (1991). A multivalent antibody may be internalized (and/or catabolized) faster than a bivalent antibody by a cell expressing an antigen to which the antibodies bind. The antibodies of the present invention can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a C_{L} domain.

Antibodies of the present invention further include single chain antibodies.

In particular embodiments, antibodies of the present invention are internalizing antibodies.

Amino acid sequence modification(s) of the antibodies described herein are contemplated. For example, it may be desirable to improve the binding affinity and/or other biological properties of the antibody. Amino acid sequence variants of the antibody may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final antibody, provided that the final construct possesses the desired characteristics. The amino acid changes also may alter post-translational processes of the antibody, such as changing the number or position of glycosylation sites. Any of the variations and modifications described above for polypeptides of the present invention may be included in antibodies of the present invention.

A useful method for identification of certain residues or regions of an antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with PSCA antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of an antibody include the fusion to the N- or C-terminus of the antibody to an enzyme (e.g., for ADEPT) or a polypeptide that increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative and non-conservative substitutions are contemplated.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody. Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and an antigen or cancer cell. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The antibody of the invention is modified with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-infection activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Antibodies of the present invention may also be modified to include an epitope tag or label, e.g., for use in purification or diagnostic applications. The invention also pertains to therapy with immunoconjugates comprising an antibody conjugated to an anti-cancer agent such as a cytotoxic agent or a growth inhibitory agent. Chemotherapeutic agents useful in the generation of such immunoconjugates have been described above.

Conjugates of an antibody and one or more small molecule toxins, such as a calicheamicin, maytansinoids, a trichothene, and CC1065, and the derivatives of these toxins that have toxin activity, are also contemplated herein.

In one preferred embodiment, an antibody (full length or fragments) of the invention is conjugated to one or more maytansinoid molecules. Maytansinoids are mitototic inhibitors that act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay.

Antibody-maytansinoid conjugates are prepared by chemically linking an antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody conjugates, including, for example, those disclosed in U.S. Pat. No. 5,208,020 or EP Patent 0 425 235 B1, and Chari et al., Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Immunoconjugates may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage. For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, Cancer Research 52: 127-131 (1992); U.S. Pat. No. 5,208,020) may be used.

Another immunoconjugate of interest comprises an antibody conjugated to one or more calicheamicin molecules. The calicheamicin family of antibiotics is capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Another drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Examples of other agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof that can be used include, e.g., diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232.

The present invention further includes an immunoconjugate formed between an antibody and a compound with nucleolytic activity (e.g., a ribonuclease or a DNA endonuclease such as a deoxyribonuclease; DNase).

For selective destruction of cancer cells, the antibody includes a highly radioactive atom. A variety of radioactive isotopes are available for the production of radioconjugated anti-PSCA antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other label is incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al. (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Alternatively, a fusion protein comprising the antibody and cytotoxic agent is made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

The antibodies of the present invention are also used in antibody dependent enzyme mediated prodrug therapy (ADET) by conjugating the antibody to a prodrug-activating enzyme which converts a prodrug (e.g., a peptidyl chemotherapeutic agent, see WO81/01145) to an active anti-cancer drug (see, e.g., WO 88/07378 and U.S. Pat. No. 4,975,278).

The enzyme component of the immunoconjugate useful for ADEPT includes any enzyme capable of acting on a prodrug in such a way so as to convert it into its more active, cytotoxic form. Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a cancer cell population.

The enzymes of this invention can be covalently bound to the antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984).

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The antibodies disclosed herein are also formulated as immunoliposomes. A “liposome” is a small vesicle composed of various types of lipids, phospholipids and/or surfactant that is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired a diameter. Fab′ fragments of the antibody of the present invention can be conjugated to the liposomes as described in Martin et al., J. Biol. Chem. 257: 286-288 (1982) via a disulfide interchange reaction. A chemotherapeutic agent is optionally contained within the liposome. See Gabizon et al., J. National Cancer Inst. 81(19)1484 (1989).

Antibodies of the present invention, or fragments thereof, may possess any of a variety of biological or functional characteristics. I

In particular embodiments, an antibody of the present invention is an antagonist antibody, which partially or fully blocks or inhibits a biological activity of a polypeptide or cell to which it specifically or preferentially binds. In other embodiments, an antibody of the present invention is a growth inhibitory antibody, which partially or fully blocks or inhibits the growth of an cancer cell to which it binds. In another embodiment, an antibody of the present invention induces apoptosis. In yet another embodiment, an antibody of the present invention induces or promotes antibody-dependent cell-mediated cytotoxicity or complement dependent cytotoxicity.

### Methods of Identifying and Producing Antibodies Specific for Cancer

The present invention provides novel methods for the identification of HuCA antibodies. These methods may be readily adapted to identify antibodies specific for other polypeptides expressed on the cell surface by cancer cells.

An exemplary cancer cell is derived from one of the following cancer types, including but not limited to, acute lymphoblastic leukemia, acute myeloid leukemia, adrenocortical carcinoma, AIDS-related cancers, AIDS-related lymphoma, anal cancer, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, mycosis fungoides, Sézary Syndrome, endometrial cancer, esophageal cancer, extracranial germ cell tumor, extragonadal germ cell tumor, eye cancer, tetraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, islet cell tumors (endocrine pancreas), Kaposi Sarcoma, kidney (renal cell) cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, non-small cell lung cancer, small cell lung cancer, non-Hodgkin lymphoma, primary central nervous system lymphoma, Waldenström macroglobulinemia, medulloblastoma, melanoma, intraocular (eye) melanoma, merkel cell carcinoma, mesothelioma malignant, mesothelioma, metastatic squamous neck cancer, mouth cancer, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine sarcoma, skin cancer (nonmelanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilm's Tumor.

In general, the methods include obtaining serum samples from patients that have been diagnosed with a cancer. These serum samples are then screened to identify those that contain antibodies specific for a particular polypeptide associated with the cancer, such as, e.g., a polypeptide specifically expressed on the surface of cancer cells, but not normal cells.

Once a patient is identified as having serum containing an antibody specific for the cancer polypeptide of interest, mononuclear and/or B cells obtained from the same patient are used to identify a cell or clone thereof that produces the antibody, using any of the methods described herein or available in the art. Once a B cell that produces the antibody is identified, cDNAs encoding the variable regions or fragments thereof of the antibody may be cloned using standard RT-PCR and primers specific for conserved antibody sequences, and subcloned in to expression vectors used for the recombinant production of monoclonal antibodies specific for the cancer polypeptide of interest.

In one embodiment, the present invention provides a method of identifying an antibody that specifically binds a cancer cell, comprising: contacting cancer cell or a cell expressing cancer antigen (e.g., protein) with a biological sample obtained from a patient having been diagnosed with cancer; determining an amount of antibody in the biological sample that binds to the cell; and comparing the amount determined with a control value, wherein if the value determined is at least two-fold greater than the control value, an antibody that specifically binds cancer cells is indicated.

Polynucleotide sequences encoding the antibodies, variable regions thereof, or antigen-binding fragments thereof may be subcloned into expression vectors for the recombinant production of HuCA antibodies. In one embodiment, this is accomplished by obtaining mononuclear cells from the patient from the serum containing the identified HuCA antibody was obtained; producing B cell clones from the mononuclear cells; inducing the B cells to become antibody-producing plasma cells; and screening the supernatants produced by the plasma cells to determine if it contains the HuCA antibody. Once a B cell clone that produces an HuCA antibody is identified, reverse-transcription polymerase chain reaction (RT-PCR) is performed to clone the DNAs encoding the variable regions or portions thereof of the HuCA antibody. These sequences are then subcloned into expression vectors suitable for the recombinant production of human HuCA antibodies. The binding specificity may be confirmed by determining the recombinant antibody's ability to bind cancer cells.

In particular embodiments of the methods described herein, B cells isolated from peripheral blood or lymph nodes are sorted, e.g., based on their being CD19 positive, and plated, e.g., as low as a single cell specificity per well, e.g., in 96, 384, or 1536 well configurations. The cells are induced to differentiate into antibody-producing cells, e.g., plasma cells, and the culture supernatants are harvested and tested for binding to cells expressing the cancer antigen on their surface using, e.g., FMAT or FACS analysis. Positive wells are then subjected to RT-PCR to amplify heavy and light chain variable regions of the IgG molecule expressed by the clonal daughter plasma cells. The resulting PCR products encoding the heavy and light chain variable regions, or portions thereof, are subcloned into human antibody expression vectors for recombinant expression. The resulting recombinant antibodies are then tested to confirm their original binding specificity and may be further tested for pan-specificity across various primary cancer cells or cancer cell lines.

Polynucleotides that encode the HuCA antibodies or portions thereof of the present invention may be isolated from cells expressing HuCA antibodies, according to methods available in the art and described herein, including amplification by polymerase chain reaction using primers specific for conserved regions of human antibody polypeptides. For example, light chain and heavy chain variable regions may be cloned from the B cell according to molecular biology techniques described in Coronella J A, Telleman P, Truong T D, Ylera F, Junghans R P. Nucleic Acids Res. 2000 Oct. 15; 28(20) or Tiller T, Meffre E, Yurasov S, Tsuiji M, Nussenzweig M C, Wardemann H. J Immunol Methods. 2008 Jan. 1; 329(1-2):112-24. In certain embodiments, polynucleotides encoding all or a region of both the heavy and light chain variable regions of the IgG molecule expressed by the clonal daughter plasma cells expressing the HuCA antibody are subcloned and sequenced. The sequence of the encoded polypeptide may be readily determined from the polynucleotide sequence. Isolated polynucleotides encoding a polypeptide of the present invention may be subcloned into an expression vector to recombinantly produce antibodies and polypeptides of the present invention, using procedures known in the art and described herein.

Binding properties of an antibody (or fragment thereof) to cancer cells or tissues may generally be determined and assessed using immunodetection methods including, for example, immunofluorescence-based assays, such as immuno-histochemistry (IHC) and/or fluorescence-activated cell sorting (FACS).

Following pre-screening of serum to identify patients (or subjects) that produce antibodies to an cancer antigen or polypeptide the methods of the present invention typically include the isolation or purification of B cells from a biological sample previously obtained from said patient or subject. The patient or subject may be currently or previously diagnosed with or suspected or having a particular cancer, or the patient or subject may be considered free or a particular disease or infection. Typically, the patient or subject is a mammal and, in particular embodiments, a human. The biological sample may be any sample that contains B cells, including but not limited to, lymph node or lymph node tissue, pleural effusions, peripheral blood, ascites, tumor tissue, or cerebrospinal fluid (CSF). In various embodiments, B cells are isolated from different types of biological samples, such as a biological sample affected by the cancer. However, it is understood that any biological sample comprising B cells may be used for any of the embodiments of the present invention.

Once isolated, the B cells are induced to produce antibodies, e.g., by culturing the B cells under conditions that support B cell proliferation or development into a plasmacyte, plasmablast, or plasma cell. The antibodies are then screened, typically using high throughput techniques, to identify an antibody that specifically binds to a target antigen, e.g., a particular tissue, cell, infectious agent, or polypeptide. In certain embodiments, the specific antigen, e.g., cell surface polypeptide bound by the antibody is not known, while in other embodiments, the antigen specifically bound by the antibody is known.

According to the present invention, B cells may be isolated from a biological sample, e.g., a tumor, tissue, peripheral blood or lymph node sample, by any means known and available in the art. B cells are typically sorted by FACS based on the presence on their surface of a B cell-specific marker, e.g., CD19, CD138, and/or surface IgG. However, other methods known in the art may be employed, such as, e.g., column purification using CD19 magnetic beads or IgG-specific magnetic beads, followed by elution from the column. However, magnetic isolation of B cells utilizing any marker may result in loss of certain B cells. Alternatively, B cells may be enriched by immunodepletion of other cell types using biotinylated antibodies including anti-CD3, anti-CD14, anti-CD16, anti-IgD, anti-IgM, anti-IgA. In certain embodiments, the isolated cells are not sorted but, instead, phicol-purified mononuclear cells isolated from tumor are directly plated to the appropriate or desired number of specificities per well.

In order to identify B cells that produce a huCA antibody, the B cells are typically plated at low density (e.g., a single cell specificity per well, 1-10 cells per well, 10-100 cells per well, 1-100 cells per well, less than 10 cells per well, or less than 100 cells per well) in multi-well or microtitre plates, e.g., in 96, 384, or 1536 well configurations. When the B cells are initially plated at a density greater than one cell per well, then the methods of the present invention may include the step of subsequently diluting cells in a well identified as producing an antigen-specific antibody, until a single cell specificity per well is achieved, thereby facilitating the identification of the B cell that produces the antigen-specific antibody. Cell supernatants or a portion thereof and/or cells may be frozen and stored for future testing and later recovery of antibody polynucleotides.

In certain embodiments, the B cells are cultured under conditions that favor the production of antibodies by the B cells. For example, the B cells may be cultured under conditions favorable for B cell proliferation and differentiation to yield antibody-producing plasmablast, plasmacytes, or plasma cells. In particular embodiments, the B cells are cultured in the presence of a B cell mitogen, such as lipopolysaccharide (LPS) or CD40 ligand. In one specific embodiment, B cells are differentiated to antibody-producing cells (See, Nature. 1991 Oct. 17; 353(6345):678-9. and Curr Opin Biotechnol. 2007 December; 18(6):523-8. Epub 2007 Dec. 11. Review.)

Cell culture supernatants or antibodies obtained therefrom may be tested for their ability to bind to a target antigen, using routine methods available in the art, including those described herein or disclosed in U.S. patent application Ser. No. 12/509,323, filed Jul. 24, 2009. In particular embodiments, culture supernatants are tested for the presence of antibodies that bind to a target antigen using high-throughput methods. For example, B cells may be cultured in multi-well microtitre dishes, such that robotic plate handlers may be used to simultaneously sample multiple cell supernatants and test for the presence of antibodies that bind to a target antigen. In particular embodiments, antigens are bound to beads, e.g., paramagnetic or latex beads) to facilitate the capture of antibody/antigen complexes. In other embodiments, antigens and antibodies are fluorescently labeled (with different labels) and FACS analysis is performed to identify the presence of antibodies that bind to target antigen. In one embodiment, antibody binding is determined using FMAT™ analysis and instrumentation (Applied Biosystems, Foster City, Calif.). FMAT™ is a fluorescence macro-confocal platform for high-throughput screening, which mix-and-read, non-radioactive assays using live cells or beads. In another embodiment the AlphaScreen™ assay method can be used. For this assay the antibody is captured from culture supernatant by an acceptor bead (for example Protein A coated) while the antigen is bound (for example through biotin derivitization) to a donor bead (for example streptavidin coated). Binding of antibody to antigen brings the donor and acceptor beads into close proximity, allowing for signal generation in an appropriate instrument.

In the context of comparing the binding of an antibody to a particular target antigen (e.g., a biological sample such as cancer tissue or cells) as compared to a control sample (e.g., a biological sample such as normal cell or tissue), in various embodiments, the antibody is considered to preferentially bind a particular target antigen if at least two-fold, at least three-fold, at least five-fold, or at least ten-fold more antibody binds to the particular target antigen as compared to the amount that binds a control sample.

The PCR products encoding the heavy and light chain variable regions or portions thereof are then subcloned into human antibody expression vectors and recombinantly expressed according to routine procedures in the art (see, e.g., U.S. Pat. No. 7,112,439). The nucleic acid molecules encoding a tumor-specific antibody or fragment thereof, as described herein, may be propagated and expressed according to any of a variety of well-known procedures for nucleic acid excision, ligation, transformation, and transfection. Thus, in certain embodiments expression of an antibody fragment may be preferred in a prokaryotic host cell, such as Escherichia coli (see, e.g., Pluckthun et al., Methods Enzymol. 178:497-515 (1989)). In certain other embodiments, expression of the antibody or an antigen-binding fragment thereof may be preferred in a eukaryotic host cell, including yeast (e.g., Saccharomyces cerevisiae, Schizosaccharomyces pombe, and Pichia pastoris); animal cells (including mammalian cells); or plant cells. Examples of suitable animal cells include, but are not limited to, myeloma, HEK293, COS, CHO, or hybridoma cells. Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the tumor-specific antibody (or fragment thereof) may be inserted. The regulatory elements will vary according to the particular host.

One or more replicable expression vectors containing a polynucleotide encoding a variable and/or constant region may be prepared and used to transform an appropriate cell line, for example, a non-producing myeloma cell line, such as a mouse NSO line or a bacteria, such as E. coli, in which production of the antibody will occur. In order to obtain efficient transcription and translation, the polynucleotide sequence in each vector should include appropriate regulatory sequences, particularly a promoter and leader sequence operatively linked to the variable domain sequence. Particular methods for producing antibodies in this way are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). While not required, in certain embodiments, regions of polynucleotides encoding the recombinant antibodies may be sequenced. DNA sequencing can be performed as described in Sanger et al. (Proc. Natl. Acad. Sci. USA 74:5463 (1977)) and the Amersham International plc sequencing handbook and including improvements thereto.

In particular embodiments, the resulting recombinant antibodies or fragments thereof are then tested to confirm their original specificity and may be further tested for pan-specificity, e.g., with related infectious agents. In particular embodiments, an antibody identified or produced according to methods described herein is tested for cell killing via antibody dependent cellular cytotoxicity (ADCC) or apoptosis, and/or well as its ability to internalize.

### Polynucleotides

The present invention, in other aspects, provides polynucleotide compositions. In preferred embodiments, these polynucleotides encode a polypeptide of the invention, e.g., a region of a variable chain of an antibody that binds to a cancer cell. Polynucleotides of the invention are single-stranded (coding or antisense) or double-stranded DNA (genomic, cDNA or synthetic) or RNA molecules. RNA molecules include, but are not limited to, HnRNA molecules, which contain introns and correspond to a DNA molecule in a one-to-one manner, and mRNA molecules, which do not contain introns. Alternatively, or in addition, coding or non-coding sequences are present within a polynucleotide of the present invention. Also alternatively, or in addition, a polynucleotide is linked to other molecules and/or support materials of the invention. Polynucleotides of the invention are used in the recombinant production of polypeptides of the invention.

Therefore, according to another aspect of the present invention, polynucleotide compositions are provided that include some or all of a polynucleotide sequence set forth in herein, complements of a polynucleotide sequence set forth in herein, and degenerate variants of a polynucleotide sequence set forth herein. Furthermore, the invention includes all polynucleotides that encode any polypeptide of the present invention.

In other related embodiments, the invention provides polynucleotide variants having substantial identity to the sequences set forth herein, for example those comprising at least 70% sequence identity, preferably at least 75%, 80%, 85%, 90%, 95%, 96%, 97%, 98%, or 99% or higher, sequence identity compared to a polynucleotide sequence of this invention, as determined using the methods described herein, (e.g., BLAST analysis using standard parameters). One skilled in this art will recognize that these values can be appropriately adjusted to determine corresponding identity of proteins encoded by two nucleotide sequences by taking into account codon degeneracy, amino acid similarity, reading frame positioning, and the like.

Typically, polynucleotide variants contain one or more substitutions, additions, deletions and/or insertions, preferably such that the immunogenic binding properties of the polypeptide encoded by the variant polynucleotide is not substantially diminished relative to a polypeptide encoded by a polynucleotide sequence specifically set forth herein. In additional embodiments, the present invention provides polynucleotide fragments comprising various lengths of contiguous stretches of sequence identical to or complementary to one or more of the sequences disclosed herein. For example, polynucleotides are provided by this invention that comprise at least about 10, 15, 20, 30, 40, 50, 75, 100, 150, 200, 300, 400, 500 or 1000 or more contiguous nucleotides of one or more of the sequences disclosed herein as well as all intermediate lengths there between. As used herein, the term “intermediate lengths” is meant to describe any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In preferred embodiments, the polypeptide encoded by the polynucleotide variant or fragment has the same binding specificity (i.e., specifically or preferentially binds to the same epitope) as the polypeptide encoded by the native polynucleotide. In certain preferred embodiments, the polynucleotides described above, e.g., polynucleotide variants, fragments and hybridizing sequences, encode polypeptides that have a level of binding activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. A nucleic acid fragment of almost any length is employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are included in many implementations of this invention.

It will be appreciated by those of ordinary skill in the art that, as a result of the degeneracy of the genetic code, there are multiple nucleotide sequences that encode a polypeptide as described herein. Some of these polynucleotides bear minimal homology to the nucleotide sequence of any native gene. Nonetheless, polynucleotides that encode a polypeptide of the present invention but which vary due to differences in codon usage are specifically contemplated by the invention. Further, alleles of the genes including the polynucleotide sequences provided herein are within the scope of the invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

In certain embodiments of the present invention, mutagenesis of the disclosed polynucleotide sequences is performed in order to alter one or more properties of the encoded polypeptide, such as its binding specificity or binding strength. Techniques for mutagenesis are well-known in the art, and are widely used to create variants of both polypeptides and polynucleotides. A mutagenesis approach, such as site-specific mutagenesis, is employed for the preparation of variants and/or derivatives of the polypeptides described herein. By this approach, specific modifications in a polypeptide sequence are made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences include the nucleotide sequence of the desired mutation, as well as a sufficient number of adjacent nucleotides, to provide a primer sequence of sufficient size and sequence complexity to form a stable duplex on both sides of the deletion junction being traversed. Mutations are employed in a selected polynucleotide sequence to improve, alter, decrease, modify, or otherwise change the properties of the polynucleotide itself, and/or alter the properties, activity, composition, stability, or primary sequence of the encoded polypeptide.

In other embodiments of the present invention, the polynucleotide sequences provided herein are used as probes or primers for nucleic acid hybridization, e.g., as PCR primers. The ability of such nucleic acid probes to specifically hybridize to a sequence of interest enable them to detect the presence of complementary sequences in a given sample. However, other uses are also encompassed by the invention, such as the use of the sequence information for the preparation of mutant species primers, or primers for use in preparing other genetic constructions. As such, nucleic acid segments of the invention that include a sequence region of at least about 15 nucleotide long contiguous sequence that has the same sequence as, or is complementary to, a 15 nucleotide long contiguous sequence disclosed herein is particularly useful. Longer contiguous identical or complementary sequences, e.g., those of about 20, 30, 40, 50, 100, 200, 500, 1000 (including all intermediate lengths) including full length sequences, and all lengths in between, are also used in certain embodiments.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting, and/or primers for use in, e.g., polymerase chain reaction (PCR). The total size of fragment, as well as the size of the complementary stretch(es), ultimately depends on the intended use or application of the particular nucleic acid segment. Smaller fragments are generally used in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 12 bases in length are generally preferred, though, in order to increase stability and selectivity of the hybrid, and thereby improve the quality and degree of specific hybrid molecules obtained. Nucleic acid molecules having gene-complementary stretches of 15 to 25 contiguous nucleotides, or even longer where desired, are generally preferred.

Hybridization probes are selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences is governed by various factors. For example, one may wish to employ primers from towards the termini of the total sequence.

Polynucleotide of the present invention, or fragments or variants thereof, are readily prepared by, for example, directly synthesizing the fragment by chemical means, as is commonly practiced using an automated oligonucleotide synthesizer. Also, fragments are obtained by application of nucleic acid reproduction technology, such as the PCR™ technology of U.S. Pat. No. 4,683,202, by introducing selected sequences into recombinant vectors for recombinant production, and by other recombinant DNA techniques generally known to those of skill in the art of molecular biology.

### Vectors, Host Cells and Recombinant Methods

The invention provides vectors and host cells comprising a nucleic acid of the present invention, as well as recombinant techniques for the production of a polypeptide of the present invention. Vectors of the invention include those capable of replication in any type of cell or organism, including, e.g., plasmids, phage, cosmids, and mini chromosomes. In various embodiments, vectors comprising a polynucleotide of the present invention are vectors suitable for propagation or replication of the polynucleotide, or vectors suitable for expressing a polypeptide of the present invention. Such vectors are known in the art and commercially available.

Polynucleotides of the present invention are synthesized, whole or in parts that are then combined, and inserted into a vector using routine molecular and cell biology techniques, including, e.g., subcloning the polynucleotide into a linearized vector using appropriate restriction sites and restriction enzymes. Polynucleotides of the present invention are amplified by polymerase chain reaction using oligonucleotide primers complementary to each strand of the polynucleotide. These primers also include restriction enzyme cleavage sites to facilitate subcloning into a vector. The replicable vector components generally include, but are not limited to, one or more of the following: a signal sequence, an origin of replication, and one or more marker or selectable genes.

In order to express a polypeptide of the present invention, the nucleotide sequences encoding the polypeptide, or functional equivalents, are inserted into an appropriate expression vector, i.e., a vector that contains the necessary elements for the transcription and translation of the inserted coding sequence. Methods well known to those skilled in the art are used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J., et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems are utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. Within one embodiment, the variable regions of a gene expressing a monoclonal antibody of interest are amplified from a hybridoma cell using nucleotide primers. These primers are synthesized by one of ordinary skill in the art, or may be purchased from commercially available sources (see, e.g., Stratagene (La Jolla, Calif.), which sells primers for amplifying mouse and human variable regions. The primers are used to amplify heavy or light chain variable regions, which are then inserted into vectors such as ImmunoZAP™ H or ImmunoZAP™ L (Stratagene), respectively. These vectors are then introduced into E. coli, yeast, or mammalian-based systems for expression. Large amounts of a single-chain protein containing a fusion of the Vh and Vl domains are produced using these methods (see Bird et al., Science 242:423-426 (1988)).

The “control elements” or “regulatory sequences” present in an expression vector are those non-translated regions of the vector, e.g., enhancers, promoters, 5′ and 3′ untranslated regions, that interact with host cellular proteins to carry out transcription and translation. Such elements may vary in their strength and specificity. Depending on the vector system and host utilized, any number of suitable transcription and translation elements, including constitutive and inducible promoters, are used.

Examples of promoters suitable for use with prokaryotic hosts include the phoa promoter, □-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also usually contain a Shine-Dalgarno sequence operably linked to the DNA encoding the polypeptide. Inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like are used.

A variety of promoter sequences are known for eukaryotes and any are used according to the present invention. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

In mammalian cell systems, promoters from mammalian genes or from mammalian viruses are generally preferred. Polypeptide expression from vectors in mammalian host cells are controlled, for example, by promoters obtained from the genomes of viruses such as polyoma virus, fowlpox virus, adenovirus (e.g., Adenovirus 2), bovine papilloma virus, avian sarcoma virus, cytomegalovirus (CMV), a retrovirus, hepatitis-B virus and most preferably Simian Virus 40 (SV40), from heterologous mammalian promoters, e.g., the actin promoter or an immunoglobulin promoter, and from heat-shock promoters, provided such promoters are compatible with the host cell systems. If it is necessary to generate a cell line that contains multiple copies of the sequence encoding a polypeptide, vectors based on SV40 or EBV may be advantageously used with an appropriate selectable marker. One example of a suitable expression vector is pcDNA-3.1 (Invitrogen, Carlsbad, Calif.), which includes a CMV promoter.

A number of viral-based expression systems are available for mammalian expression of polypeptides. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus that is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

In bacterial systems, any of a number of expression vectors are selected depending upon the use intended for the expressed polypeptide. For example, when large quantities are desired, vectors that direct high level expression of fusion proteins that are readily purified are used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase, so that a hybrid protein is produced; pIN vectors (Van Heeke, G. and S. M. Schuster (1989) J. Biol. Chem. 264:5503-5509); and the like. pGEX Vectors (Promega, Madison, Wis.) are also used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH are used. Examples of other suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. For reviews, see Ausubel et al. (supra) and Grant et al. (1987) Methods Enzymol. 153:516-544. Other yeast promoters that are inducible promoters having the additional advantage of transcription controlled by growth conditions include the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

In cases where plant expression vectors are used, the expression of sequences encoding polypeptides are driven by any of a number of promoters. For example, viral promoters such as the 35S and 19S promoters of CaMV are used alone or in combination with the omega leader sequence from TMV (Takamatsu, N. (1987) EMBO J. 6:307-311. Alternatively, plant promoters such as the small subunit of RUBISCO or heat shock promoters are used (Coruzzi, G. et al. (1984) EMBO J. 3:1671-1680; Broglie, R. et al. (1984) Science 224:838-843; and Winter, J., et al. (1991) Results Probl. Cell Differ. 17:85-105). These constructs can be introduced into plant cells by direct DNA transformation or pathogen-mediated transfection. Such techniques are described in a number of generally available reviews (see, e.g., Hobbs, S. or Murry, L. E. in McGraw Hill Yearbook of Science and Technology (1992) McGraw Hill, New York, N.Y.; pp. 191-196).

An insect system is also used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide are cloned into a non-essential region of the virus, such as the polyhedrin gene, and placed under control of the polyhedrin promoter. Successful insertion of the polypeptide-encoding sequence renders the polyhedrin gene inactive and produce recombinant virus lacking coat protein. The recombinant viruses are then used to infect, for example, S. frugiperda cells or Trichoplusia larvae, in which the polypeptide of interest is expressed (Engelhard, E. K. et al. (1994) Proc. Natl. Acad. Sci. 91:3224-3227).

Specific initiation signals are also used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon are provided. Furthermore, the initiation codon is in the correct reading frame to ensure correct translation of the inserted polynucleotide. Exogenous translational elements and initiation codons are of various origins, both natural and synthetic.

Transcription of a DNA encoding a polypeptide of the invention is often increased by inserting an enhancer sequence into the vector. Many enhancer sequences are known, including, e.g., those identified in genes encoding globin, elastase, albumin, α-fetoprotein, and insulin. Typically, however, an enhancer from a eukaryotic cell virus is used. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer is spliced into the vector at a position 5′ or 3′ to the polypeptide-encoding sequence, but is preferably located at a site 5′ from the promoter.

Expression vectors used in eukaryotic host cells (yeast, fungi, insect, plant, animal, human, or nucleated cells from other multicellular organisms) typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-PSCA antibody. One useful transcription termination component is the bovine growth hormone polyadenylation region. See WO94/11026 and the expression vector disclosed therein.

Suitable host cells for cloning or expressing the DNA in the vectors herein are the prokaryote, yeast, plant or higher eukaryote cells described above. Examples of suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Saccharomyces cerevisiae, or common baker's yeast, is the most commonly used among lower eukaryotic host microorganisms. However, a number of other genera, species, and strains are commonly available and used herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris. (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

In certain embodiments, a host cell strain is chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing that cleaves a “prepro” form of the protein is also used to facilitate correct insertion, folding and/or function. Different host cells such as CHO, COS, HeLa, MDCK, HEK293, and WI38, which have specific cellular machinery and characteristic mechanisms for such post-translational activities, are chosen to ensure the correct modification and processing of the foreign protein.

Methods and reagents specifically adapted for the expression of antibodies or fragments thereof are also known and available in the art, including those described, e.g., in U.S. Pat. Nos. 4,816,567 and 6,331,415. In various embodiments, antibody heavy and light chains, or fragments thereof, are expressed from the same or separate expression vectors. In one embodiment, both chains are expressed in the same cell, thereby facilitating the formation of a functional antibody or fragment thereof.

Full length antibody, antibody fragments, and antibody fusion proteins are produced in bacteria, in particular when glycosylation and Fc effector function are not needed, such as when the therapeutic antibody is conjugated to a cytotoxic agent (e.g., a toxin) and the immunoconjugate by itself shows effectiveness in cancer cell destruction. For expression of antibody fragments and polypeptides in bacteria, see, e.g., U.S. Pat. Nos. 5,648,237, 5,789,199, and 5,840,523, which describes translation initiation region (TIR) and signal sequences for optimizing expression and secretion. After expression, the antibody is isolated from the E. coli cell paste in a soluble fraction and can be purified through, e.g., a protein A or G column depending on the isotype. Final purification can be carried out using a process similar to that used for purifying antibody expressed e.g., in CHO cells.

Suitable host cells for the expression of glycosylated polypeptides and antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopicius (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses are used as the virus herein according to the present invention, particularly for transfection of Spodoptera frugiperda cells. Plant cell cultures of cotton, corn, potato, soybean, petunia, tomato, and tobacco are also utilized as hosts.

Methods of propagation of antibody polypeptides and fragments thereof in vertebrate cells in culture (tissue culture) are encompassed by the invention. Examples of mammalian host cell lines used in the methods of the invention are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for polypeptide production and cultured in conventional nutrient media modified as appropriate for inducing promoters, selecting transformants, or amplifying the genes encoding the desired sequences.

For long-term, high-yield production of recombinant proteins, stable expression is generally preferred. For example, cell lines that stably express a polynucleotide of interest are transformed using expression vectors that contain viral origins of replication and/or endogenous expression elements and a selectable marker gene on the same or on a separate vector. Following the introduction of the vector, cells are allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells are proliferated using tissue culture techniques appropriate to the cell type.

A plurality of selection systems are used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes that are employed in tk⁻ or aprt⁻ cells, respectively. Also, antimetabolite, antibiotic or herbicide resistance is used as the basis for selection; for example, dhfr, which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al. (1981) J. Mol. Biol. 150:1-14); and als or pat, which confer resistance to chlorsulfuron and phosphinotricin acetyltransferase, respectively (Murry, supra). Additional selectable genes have been described. For example, trpB allows cells to utilize indole in place of tryptophan, and hisD allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression is confirmed. For example, if the sequence encoding a polypeptide is inserted within a marker gene sequence, recombinant cells containing sequences are identified by the absence of marker gene function. Alternatively, a marker gene is placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence are identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Nonlimiting examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide is preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

Various labels and conjugation techniques are known by those skilled in the art and are used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof are cloned into a vector for the production of an mRNA probe. Such vectors are known in the art, are commercially available, and are used to synthesize RNA probes in vitro by addition of an appropriate RNA polymerase such as T7, T3, or SP6 and labeled nucleotides. These procedures are conducted using a variety of commercially available kits. Suitable reporter molecules or labels, which are used include, but are not limited to, radionuclides, enzymes, fluorescent, chemiluminescent, or chromogenic agents as well as substrates, cofactors, inhibitors, magnetic particles, and the like.

The polypeptide produced by a recombinant cell is secreted or contained intracellularly depending on the sequence and/or the vector used. Expression vectors containing polynucleotides of the invention are designed to contain signal sequences that direct secretion of the encoded polypeptide through a prokaryotic or eukaryotic cell membrane.

In certain embodiments, a polypeptide of the invention is produced as a fusion polypeptide further including a polypeptide domain that facilitates purification of soluble proteins. Such purification-facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Amgen, Seattle, Wash.). The inclusion of cleavable linker sequences such as those specific for Factor XA or enterokinase (Invitrogen. San Diego, Calif.) between the purification domain and the encoded polypeptide are used to facilitate purification. An exemplary expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors used for producing fusion proteins is provided in Kroll, D. J. et al. (1993; DNA Cell Biol. 12:441-453).

In certain embodiments, a polypeptide of the present invention is fused with a heterologous polypeptide, which may be a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells, the signal sequence is selected, for example, from the group of the alkaline phosphatase, penicillinase, 1 pp, or heat-stable enterotoxin II leaders. For yeast secretion, the signal sequence is selected from, e.g., the yeast invertase leader, a factor leader (including Saccharomyces and Kluyveromyces a factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

When using recombinant techniques, the polypeptide or antibody is produced intracellularly, in the periplasmic space, or directly secreted into the medium. If the polypeptide or antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris is removed by centrifugation. Where the polypeptide or antibody is secreted into the medium, supernatants from such expression systems are generally first concentrated using a commercially available protein concentration filter, for example, an Amicon or Millipore Pellicon ultrafiltration unit. Optionally, a protease inhibitor such as PMSF is included in any of the foregoing steps to inhibit proteolysis and antibiotics are included to prevent the growth of adventitious contaminants.

The polypeptide or antibody composition prepared from the cells are purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the polypeptide or antibody. Protein A is used to purify antibodies or fragments thereof that are based on human γ₁, γ₂, or γ₄ heavy chains (Lindmark et al., J. Immunol. Meth. 62:1-13 (1983)). Protein G is recommended for all mouse isotypes and for human γ₃ (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the polypeptide or antibody comprises a C_{H} 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the polypeptide or antibody to be recovered. Following any preliminary purification step(s), the mixture comprising the polypeptide or antibody of interest and contaminants are subjected to low pH hydrophobic interaction chromatography using an elution buffer at a pH between about 2.5-4.5, preferably performed at low salt concentrations (e.g., from about 0-0.25M salt).

### Pharmaceutical Compositions

The invention further includes pharmaceutical formulations including a polypeptide, antibody, or modulator of the present invention, at a desired degree of purity, and a pharmaceutically acceptable carrier, excipient, or stabilizer (Remington's Pharmaceutical Sciences 16th edition, Osol, A. Ed. (1980)). In certain embodiments, pharmaceutical formulations are prepared to enhance the stability of the polypeptide or antibody during storage, e.g., in the form of lyophilized formulations or aqueous solutions. Acceptable carriers, excipients, or stabilizers are nontoxic to recipients at the dosages and concentrations employed, and include, e.g., buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g. Zn-protein complexes); and/or non-ionic surfactants such as TWEEN™, PLURONICS™ or polyethylene glycol (PEG). In certain embodiments, the therapeutic formulation preferably comprises the polypeptide or antibody at a concentration of between 5-200 mg/ml, preferably between 10-100 mg/ml.

The formulations herein also contain one or more additional therapeutic agents suitable for the treatment of the particular indication, e.g., cancer being treated, or to prevent undesired side-effects. Preferably, the additional therapeutic agent has an activity complementary to the polypeptide or antibody of the resent invention, and the two do not adversely affect each other. For example, in addition to the polypeptide or antibody of the invention, an additional or second antibody, or a chemotherapeutic agent is added to the formulation. Such molecules are suitably present in the pharmaceutical formulation in amounts that are effective for the purpose intended.

The active ingredients, e.g., polypeptides and antibodies of the invention and other therapeutic agents, are also entrapped in microcapsules prepared, for example, by coacervation techniques or by interfacial polymerization, for example, hydroxymethylcellulose or gelatin-microcapsules and polymethylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations are prepared. Suitable examples of sustained-release preparations include, but are not limited to, semi-permeable matrices of solid hydrophobic polymers containing the antibody, which matrices are in the form of shaped articles, e.g., films, or microcapsules. Nonlimiting examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxyburyric acid.

Formulations to be used for in vivo administration are preferably sterile. This is readily accomplished by filtration through sterile filtration membranes.

### Diagnostic Uses

Antibodies and fragments thereof, and therapeutic compositions, of the invention specifically bind or preferentially bind to cancer cells or tissue, as compared to normal control cells and tissue. Thus, these anti-cancer antibodies are used to detect cancer cells or tissues in a patient, biological sample, or cell population, using any of a variety of diagnostic and prognostic methods, including those described herein. The ability of an anti-cancer antibody to detect cancer cells depends upon its binding specificity, which is readily determined by testing its ability to bind to cancer cells or tissues obtained from different patients, and/or from patients having different types of cancer.

The antibodies described herein detect cancer cells derived from the cancers including, but not limited to, acute lymphoblastic leukemia, acute myeloid leukemia, adrenocortical carcinoma, AIDS-related cancers, AIDS-related lymphoma, anal cancer, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, mycosis fungoides, Sézary Syndrome, endometrial cancer, esophageal cancer, extracranial germ cell tumor, extragonadal germ cell tumor, eye cancer, tetraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, islet cell tumors (endocrine pancreas), Kaposi Sarcoma, kidney (renal cell) cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, non-small cell lung cancer, small cell lung cancer, non-Hodgkin lymphoma, primary central nervous system lymphoma, Waldenström macroglobulinemia, medulloblastoma, melanoma, intraocular (eye) melanoma, merkel cell carcinoma, mesothelioma malignant, mesothelioma, metastatic squamous neck cancer, mouth cancer, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine sarcoma, skin cancer (nonmelanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilms Tumor.

In particular, the invention provides antibodies for the detection of cancer cells derived from endocrine (adrenal), brain, esophageal, breast, colon, kidney, liver, lung, muscle (smooth, striated, and skeletal), intestinal (particularly, the small intestine), nerve, reproductive (e.g. ovarian and testicular), or connective tissue (sarcomas) or organs.

Diagnostic methods generally involve contacting a biological sample obtained from a patient, such as, e.g., blood, serum, saliva, urine, sputum, a cell swab sample, or a tissue biopsy, with an HuCA antibody and determining whether the antibody preferentially binds to the sample as compared to a control sample or predetermined cut-off value, thereby indicating the presence of cancer cells. In particular embodiments, at least two-fold, three-fold, or five-fold more HuCA antibody binds to an cancer cell as compared to an appropriate control normal cell or tissue sample. A pre-determined cut-off value is determined, e.g., by averaging the amount of HuCA antibody that binds to several different appropriate control samples under the same conditions used to perform the diagnostic assay of the biological sample being tested.

Bound antibody is detected using procedures described herein and known in the art. In certain embodiments, diagnostic methods of the invention are practiced using HuCA antibodies that are conjugated to a detectable label, e.g., a fluorophore, to facilitate detection of bound antibody. However, they are also practiced using methods of secondary detection of the HuCA antibody. These include, for example, RIA, ELISA, precipitation, agglutination, complement fixation and immuno-fluorescence.

In certain procedures, the HuCA antibodies are labeled. The label is detected directly. Exemplary labels that are detected directly include, but are not limited to, radiolabels and fluorochromes. Alternatively, or in addition, labels are moieties, such as enzymes, that must be reacted or derivatized to be detected. Nonlimiting examples of isotope labels are ⁹⁹Tc, ¹⁴C, ¹³¹I, ¹²⁵I, ³H, ³²P and ³⁵S. Fluorescent materials that are used include, but are not limited to, for example, fluorescein and its derivatives, rhodamine and its derivatives, auramine, dansyl, umbelliferone, luciferia, 2,3-dihydrophthalazinediones, horseradish peroxidase, alkaline phosphatase, lysozyme, and glucose-6-phosphate dehydrogenase.

An enzyme label is detected by any of the currently utilized colorimetric, spectrophotometric, fluorospectro-photometric or gasometric techniques. Many enzymes which are used in these procedures are known and utilized by the methods of the invention. Nonlimiting examples are peroxidase, alkaline phosphatase, β-glucuronidase, β-D-glucosidase, β-D-galactosidase, urease, glucose oxidase plus peroxidase, galactose oxidase plus peroxidase and acid phosphatase.

The antibodies are tagged with such labels by known methods. For instance, coupling agents such as aldehydes, carbodiimides, dimaleimide, imidates, succinimides, bid-diazotized benzadine and the like are used to tag the antibodies with the above-described fluorescent, chemiluminescent, and enzyme labels. An enzyme is typically combined with an antibody using bridging molecules such as carbodiimides, periodate, diisocyanates, glutaraldehyde and the like. Various labeling techniques are described in Morrison, Methods in Enzymology 32b, 103 (1974), Syvanen et al., J. Biol. Chem. 284, 3762 (1973) and Bolton and Hunter, Biochem J. 133, 529 (1973).

HuCA antibodies of the present invention are capable of differentiating between patients with and patients without cancer, and determining whether or not a patient has cancer using the representative assays provided herein. According to one method, a biological sample is obtained from a patient suspected of having or known to have an cancer. In preferred embodiments, the biological sample includes cells from the patient. The sample is contacted with an HuCA antibody, e.g., for a time and under conditions sufficient to allow the HuCA antibody to bind to cancer cells present in the sample. For instance, the sample is contacted with an HuCA antibody for 10 seconds, 30 seconds, 1 minute, 5 minutes, 10 minutes, 30 minutes, 1 hour, 6 hours, 12 hours, 24 hours, 3 days or any point in between. The amount of bound HuCA antibody is determined and compared to a control value, which may be, e.g., a pre-determined value or a value determined from normal tissue sample. An increased amount of antibody bound to the patient sample as compared to the control sample is indicative of the presence of cancer cells in the patient sample.

In a related method, a biological sample obtained from a patient is contacted with an HuCA antibody for a time and under conditions sufficient to allow the antibody to bind to cancer cells. Bound antibody is then detected, and the presence of bound antibody indicates that the sample contains cancer cells. This embodiment is particularly useful when the HuCA antibody does not bind normal cells at a detectable level.

Different HuCA antibodies possess different binding and specificity characteristics. Depending upon these characteristics, particular HuCA antibodies are used to detect the presence of one or more types of cancer. For example, certain antibodies bind specifically to only one or several types of cancers, whereas others bind to all or a majority of different cancers. Antibodies specific for only one cancer are used to identify a particular cancer

In certain embodiments, antibodies that bind to a cancer cell preferably generate a signal indicating the presence of an infection in at least about 20% of patients with the infection being detected, more preferably at least about 30% of patients. Alternatively, or in addition, the antibody generates a negative signal indicating the absence of the cancer in at least about 90% of individuals without the cancer being detected. Each antibody satisfies the above criteria; however, antibodies of the present invention are used in combination to improve sensitivity.

The present invention also includes kits useful in performing diagnostic and prognostic assays using the antibodies of the present invention. Kits of the invention include a suitable container comprising a HuCA antibody of the invention in either labeled or unlabeled form. In addition, when the antibody is supplied in a labeled form suitable for an indirect binding assay, the kit further includes reagents for performing the appropriate indirect assay. For example, the kit includes one or more suitable containers including enzyme substrates or derivatizing agents, depending on the nature of the label. Control samples and/or instructions are also included.

### Therapeutic/Prophylactic Uses

Passive immunization has proven to be an effective and safe strategy for the prevention and treatment of viral diseases. (See Keller et al., Clin. Microbiol. Rev. 13:602-14 (2000); Casadevall, Nat. Biotechnol. 20:114 (2002); Shibata et al., Nat. Med. 5:204-10 (1999); and Igarashi et al., Nat. Med. 5:211-16 (1999), each of which are incorporated herein by reference)).

HuCA antibodies and fragments thereof, and therapeutic compositions, of the invention specifically bind or preferentially bind to cancer cells, as compared to normal control cells and tissue. Thus, these HuCA antibodies are used to selectively target cancer cells or tissues in a patient, biological sample, or cell population. In light of the specific binding properties of these antibodies, the present invention provides methods of regulating (e.g., inhibiting) the growth of cancer cells, methods of killing cancer cells, and methods of inducing apoptosis of cancer cells. These methods include contacting a cancer cell with an HuCA antibody of the invention. These methods are practiced in vitro, ex vivo, and in vivo.

In various embodiments, antibodies of the invention are intrinsically, therapeutically active. Alternatively, or in addition, antibodies of the invention are conjugated to a cytotoxic agent or growth inhibitory agent, e.g., a radioisotope or toxin, that is used in treating cancer cells bound or contacted by the antibody.

In one embodiment, the invention provides methods of treating or preventing cancer in a patient, including the steps of providing an HuCA antibody of the invention to a patient diagnosed with, at risk of developing, or suspected of having cancer. The methods of the invention are used in the first-line treatment, follow-on treatment, or in the treatment of a relapsed or refractory infection. Treatment with an antibody of the invention is a stand alone treatment. Alternatively, treatment with an antibody of the invention is one component or phase of a combination therapy regime, in which one or more additional therapeutic agents are also used to treat the patient.

Administration of a prophylactic agent can occur prior to the manifestation of symptoms characteristic cancer, such that a disease or disorder is prevented or, alternatively, delayed in its progression.

In another aspect, the antibody provides a therapeutic benefit. In various aspects, a therapeutic benefit includes reducing or decreasing progression, severity, frequency, duration or probability of one or more symptoms or complications of cancer. Still another aspect, a therapeutic benefit includes hastening or accelerating a subject's recovery from cancer.

Methods for protecting a subject from cancer or decreasing susceptibility of a subject to cancer are additionally provided. In one embodiment, a method includes administering to the subject an amount of huCA antibody t to protect the subject from cancer or effective to decrease susceptibility of the subject developing cancer

Optionally, the subject is further administered with a second agent such as, but not limited to, a second anti-cancer antibody, radiation, or a chemotherapeutic agent

For in vivo treatment of human and non-human patients, the patient is usually administered or provided a pharmaceutical formulation including a HuCA antibody of the invention. When used for in vivo therapy, the antibodies of the invention are administered to the patient in therapeutically effective amounts (i.e., amounts that eliminate or reduce the patient's tumor urden). The antibodies are administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. The antibodies may be administered parenterally, when possible, at the target cell site, or intravenously. Intravenous or subcutaneous administration of the antibody is preferred in certain embodiments. Therapeutic compositions of the invention are administered to a patient or subject systemically, parenterally, or locally.

For parenteral administration, the antibodies are formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate are also used. Liposomes are used as carriers. The vehicle contains minor amounts of additives such as substances that enhance isotonicity and chemical stability, e.g., buffers and preservatives. The antibodies are typically formulated in such vehicles at concentrations of about 1 mg/ml to 10 mg/ml.

The dose and dosage regimen depends upon a variety of factors readily determined by a physician, such as the nature of the infection and the characteristics of the particular cytotoxic agent or growth inhibitory agent conjugated to the antibody (when used), e.g., its therapeutic index, the patient, and the patient's history. Generally, a therapeutically effective amount of an antibody is administered to a patient. In particular embodiments, the amount of antibody administered is in the range of about 0.1 mg/kg to about 50 mg/kg of patient body weight. Depending on the type and severity of the infection, about 0.1 mg/kg to about 50 mg/kg body weight (e.g., about 0.1-15 mg/kg/dose) of antibody is an initial candidate dosage for administration to the patient, whether, for example, by one or more separate administrations, or by continuous infusion. The progress of this therapy is readily monitored by conventional methods and assays and based on criteria known to the physician or other persons of skill in the art.

In one particular embodiment, an immunoconjugate including the antibody conjugated with a cytotoxic agent is administered to the patient. Preferably, the immunoconjugate is internalized by the cell, resulting in increased therapeutic efficacy of the immunoconjugate in killing the cell to which it binds. In one embodiment, the cytotoxic agent targets or interferes with the nucleic acid in the cancer cell. Examples of such cytotoxic agents are described above and include, but are not limited to, maytansinoids, calicheamicins, ribonucleases and DNA endonucleases.

Other therapeutic regimens are combined with the administration of the HuCA antibody of the present invention. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

In certain embodiments, it is desirable to combine administration of an antibody of the invention with another antibody directed against another antigen associated with the cancer.

Aside from administration of the antibody protein to the patient, the invention provides methods of administration of the antibody by gene therapy. Such administration of nucleic acid encoding the antibody is encompassed by the expression “administering a therapeutically effective amount of an antibody”. See, for example, PCT Patent Application Publication WO96/07321 concerning the use of gene therapy to generate intracellular antibodies.

All of the above U.S. patents, U.S. patent application publications, U.S. patent applications, foreign patents, foreign patent applications and non-patent publications referred to in this specification and/or listed in the Application Data Sheetare incorporated herein by reference, in their entirety.

### EXAMPLES

### Example 1

### Identification of Tumor-Specific Antibodies

Tumor specific antibodies were identified in serum obtained from patients with either ovarian or breast cancer. The serum was tested for reactivity against multiple breast and ovarian cancer cell lines. Briefly, cells from 8 different cell lines were grown in culture including: breast adenocarcinoma cell lines BT474, SKBR3, MCF-7, 893 and MDA-MB-231; ovarian adenocarcinoma cell lines SKOV3 and OVCAR3; embryonic fibroblast line HEK 293E. Cells were harvested by centrifugation and resuspended in media containing varied combination of three different intracellular dyes (Celltracker Blue CMAC (Invitrogen, C2110), Celltracker Green CMFDA (Invitrogen, C7025) and Celltracker Orange CMRA (Invitrogen, C34551)) in varied combinations to uniquely mark the cell lines. The stained cells were then mixed and aliquoted to 96 well round bottom plates. Serum or plasma samples were diluted in FACS buffer, added to cells and incubated. After subsequent washing, antibodies binding to the cells were stained using anti-human IgG (FC)-biotin and streptavidin-AlexaFluor-647. Finally, the cell viability dye 7-AAD was added and individual samples analyzed by FACS.

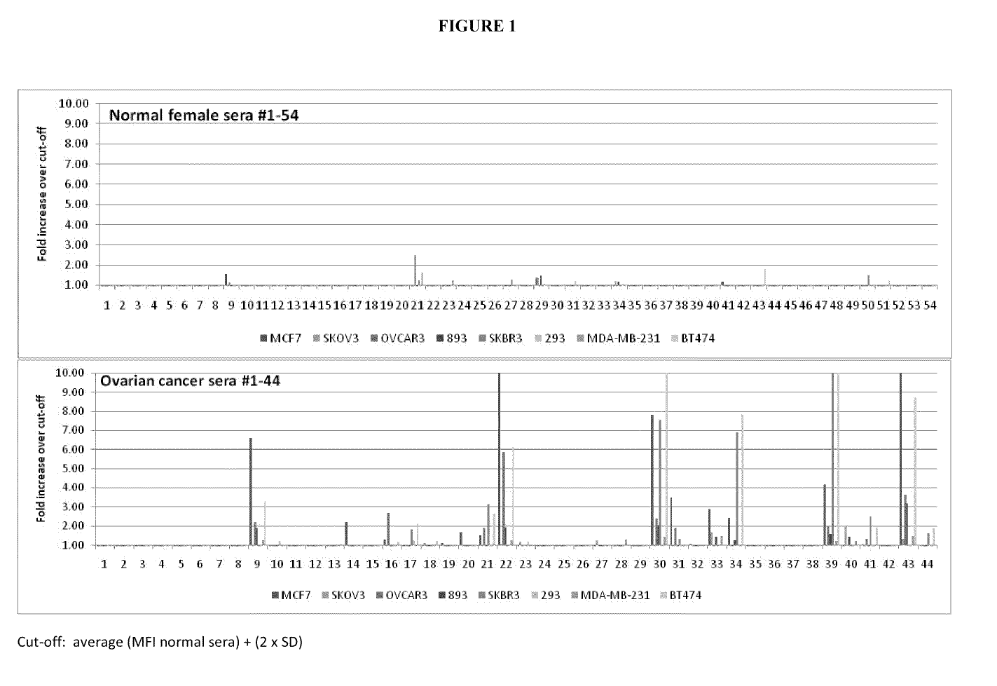

As shown in FIG. 1, serum obtained from many ovarian cancer patients reacted with various cancer cell lines but not the control line HEK293. In contrast, sera from normal subjects showed very little to no activity with any cell line. This data was used to establish an MFI cut-off value for comparative analysis of individual samples; the average MFI for all normal sera plus 2 standard deviations.

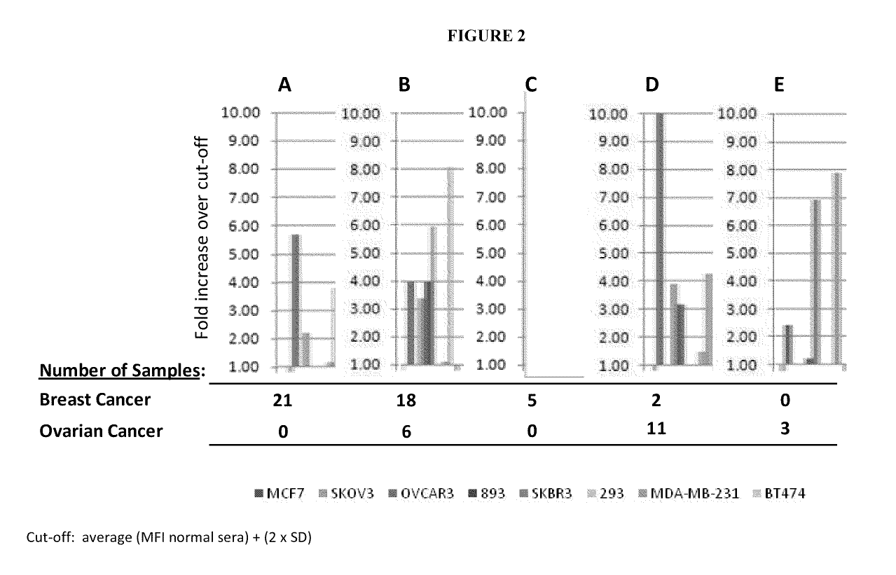

Moreover, the reactivity of the sera obtained from the patient population showed distinct patterns of reactivity against the various cancer cell lines (FIG. 2).

TABLE 1

| | Breast | Ovarian | | |
|---|---|---|---|---|
| Pat- | Cancer | Cancer | Stain Positive | Stain Negative |
| tern | Samples | Samples | Cell Lines | Cell Lines |
| A | 21 | 0 | MCF7, OVCAR3, | SKOV3, 893, SKBR3, |
| | | | BT474 | 293, MDA-MB-231 |
| B | 18 | 6 | MCF7, OVCAR3, | SKOV3, 293, MDA- |
| | | | 893, SKBR3, BT474 | MB-231, |
| C | 5 | 0 | 893, SKBR3, BT474 | MCF7, SKOV3, |
| | | | | OVCAR3, 293, MDA- |
| | | | | MB-231 |
| D | 2 | 11 | MCF7, OVCAR3, | SKOV3, SKBR3, 293, |
| | | | 893, BT474 | MDA-MB-231 |
| E | 0 | 3 | MCF7, SKBR3, | SKOV3, OVCAR3, |
| | | | BT474 | 893, 293, MDA- |
| | | | | MB-231 |

For example, seen in sera from both cancer types (18 breast cancer sera and 6 ovarian cancer sera) was a patterns of positive staining with MCF7, OVCAR3, 893, SKBR3 and BT474 cell lines and negative staining with SKOV3, 293, and MDA-MB-231 cell lines (Table 1). In addition, some cell line reactivity patterns were distinct for the different cancer types. For example, 21 breast cancer sera and no ovarian cancer sera showed the pattern of positive staining with MCF7, OVCAR3 and BT474 cell lines and negative staining with the other cell lines.

### Example 2

### Isolation of Tumor-Specific Antibodies

Tumor-specific B cells were isolated from a patient diagnosed with breast or ovarian cancer. Memory B cells (surface IgG+B cells) were enriched from PBMC by immunodepletion of other cell types using biotinylated antibodies including anti-CD3, anti-CD14, anti-CD16, anti-IgD, anti-IgM, anti-IgA. B-cells were then plated in 384-well format and cultured to produce antibody. After culture, the cell and supernatant components were separated and the cells lysed and stored frozen to preserve the mRNA component. The antibody-containing supernatants were transferred to 384-well plates for subsequent screening. Supernatants from cultured B cells isolated from the tumor sample were tested for their ability to bind breast cancer cells and normal tissue using high content imaging. Briefly, an aliquot of supernatant was incubated in a 384-well plate with a target cell line for which serum reactivity was observed (i.e., SK-BR-3 or OVCAR-3). The binding of antibody to cells was detected by secondary antibody staining with a fluorescently labeled anti-human-Fc. After washing, cells were further stained for viability and imaged using an In Cell imager. Wells showing positive antibody staining of viable cells were chosen for subsequent confirmation staining using FACS analysis. From reactive B-cell supernatants, recombinant human antibodies were prepared by cloning polynucleotides encoding the light and heavy chain IgG variable regions of the identified tumor specific antibody into expression vectors containing the corresponding constant regions. The resulting antibodies were recombinantly expressed in 293 cells and isolated from cell culture supernatant.

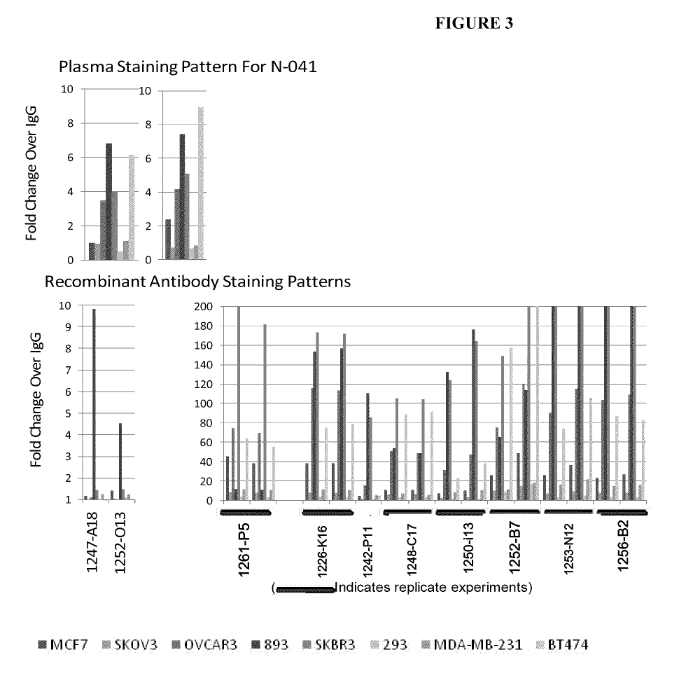

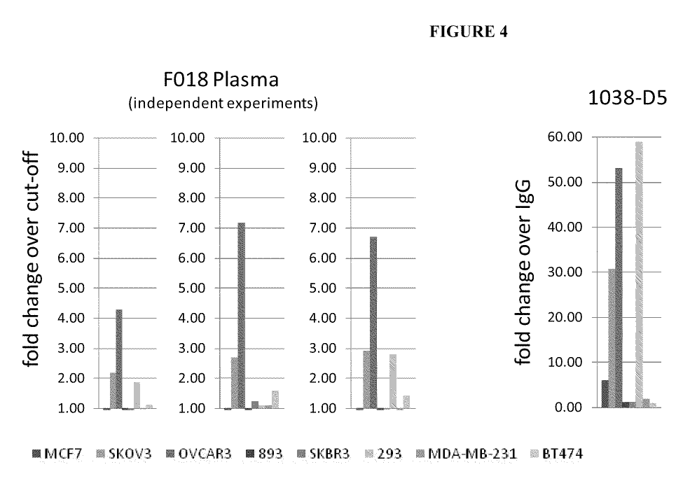

The antibodies designated 1247_A18, 1252_O13, 1226_K16, 1242_P11, 1253_N12, 1256_B2, 1250_I13, 1252_B7, 1248_C17, 1261_P5 were all derived from the same patient PBMC sample, designated N041, and specifically bound to breast and ovarian tumor cell lines but not the 293 control cell line (FIG. 3). Staining experiments with recombinant antibodies included a human IgG control that was used to normalize the data. Three different cell line staining patterns were observed among recombinant antibodies from donor N041. The reactivity pattern seen in the plasma assay was recapitulated in the staining pattern observed for 1226_K16, 1242_P11, 1256_B2, 1250_I13, 1252_B7, 1248_C17, and 1253-N12. A unique pattern of reactivity, staining only 893 cells, was seen with antibodies 1247_A18 and 1252_O13. Antibody 1261_P5 had a staining pattern similar to that seen in the plasma of donor N041 with the exception that 893 cells were not stained. Antibody 1038_D5 (TCN-445) was cloned from donor F018 and also recapitulates the staining pattern observed with plasma (FIG. 4). Antibody 1061_I16 (TCN-462) was cloned from donor F017 and strongly stained cell line OVCAR-3 as seen observed with the plasma but also strongly stained SKOV3 cells.

### Example 3

### Saturation Binding and Affinity Determination

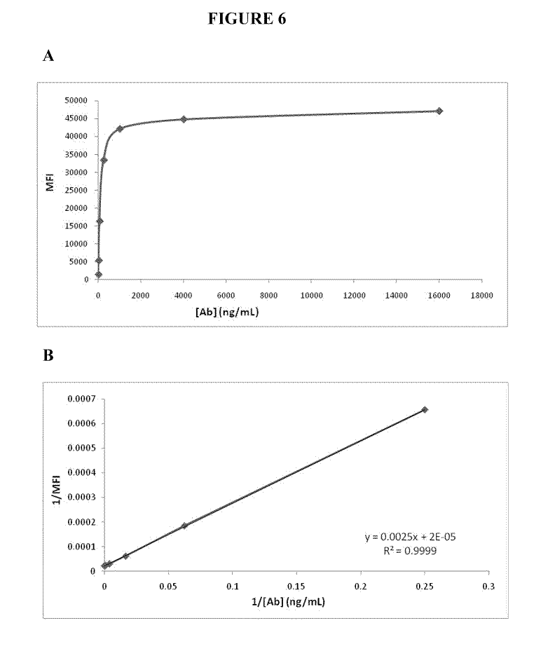

The affinity of individual antibodies for a target cell line was determined by saturation binding. Cells expressing the antibody target were aliquoted in 96-well plate and incubated with antibody at concentrations ranging from 1 mg/ml to 0.25 ng/ml. After washing, and staining with a secondary antibody, samples were analyzed by FACS. The mean fluorescence intensity (MFI) was determined at each concentration of antibody and the saturation curve for antibody 1038_D5 (TCN-445) is shown in FIG. 6A. The affinity was approximated from the double-reciprocal plot (shown for 1038_D5 (TCN-445) in FIG. 6B) according to the method described by Benedict, et al. (Benedict C A, MacKrell A J, Anderson W F. J Immunol Methods. 1997 Feb. 28; 201(2):223-31). Affinities determined using this method are tabulated below.

TABLE 2

| Antibody | Cell Line | Rel. Affinity (nM) |
|---|---|---|
| 1038-D5 (TCN-445) | OVCAR-3 | 0.8 |
| 1250-I13 | SK-BR-3 | 1.1 |
| 1252-B7 | SK-BR-3 | 2.2 |
| 1248-C17 | OVCAR-3 | 1.8 |
| 1253-N12 | SK-BR-3 | 1.1 |
| 1256-B2 | SK-BR-3 | 1.2 |
| 1226-K16 | OVCAR-3 | 1.6 |
| 1242-P11 | OVCAR-3 | 2.0 |

### Example 4

### Cytotoxic Activity of Tumor Specific Antibody 1038_D5 (TCN-445)

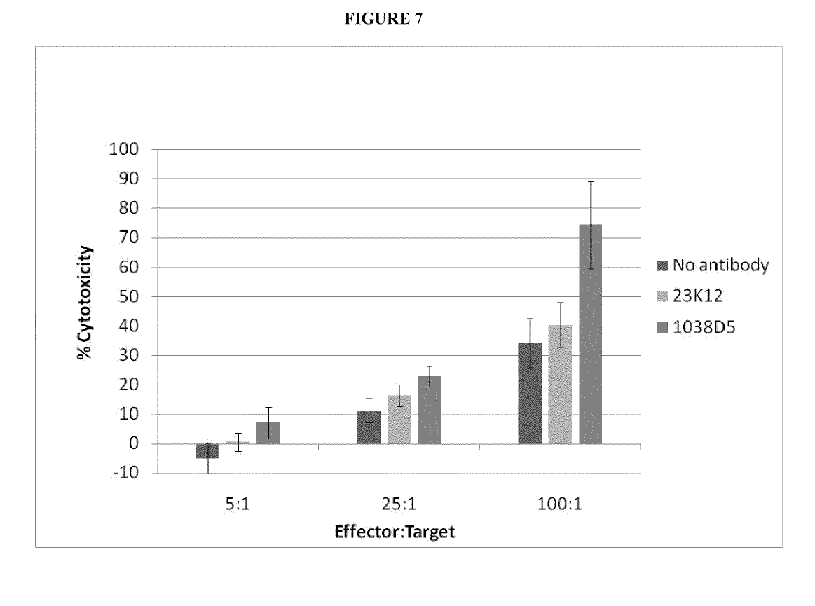

The ability of the tumor antibody 1038_D5 (TCN-445) to mediate the killing of tumor cells was examined using a antibody-mediated cell-mediated cytoxicity (ADCC) assay. The ovarian cancer cell line OVCAR3 cells were aliquoted at 10,000 cells/well in a 96-well plate in the presence of 5, 25 or 100-fold excess of PBMC and 200 ng/ml antibody. After incubation for seven hours, cell killing was assessed using the CytoTox-Glo assay (Promega). The percent ADCC was calculated as: % ADCC={[(Sample RLU)−(Target Spont.Release)]/[(Max release)−(Target Spont.Release)]}×100. As shown in FIG. 7, antibody 1038_D5 (TCN-445) induced significant ADCC of OVCAR-3 cells as compared to no antibody or matched IgG1 isotype control, 23K12, specific for an irrelevant antigen (approximately 55% maximum killing). These results demonstrate that antibody 1038_D5 (TCN-445) may be used therapeutically to induce killing of tumor cells.

### Example 5

### Immunohistochemistry

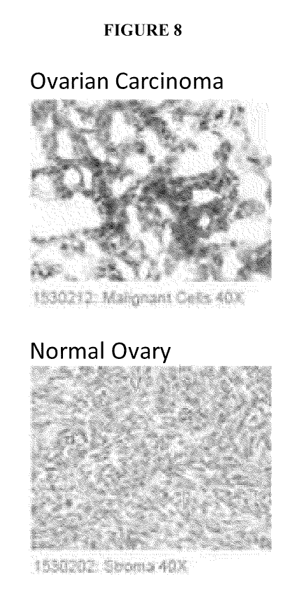

The antibody 1038_D5 (TCN-445) was tested for its ability to bind a variety of different tumor and normal cells. The tumor specificity of antibody 1038_D5 (TCN-445) was examined using standard immunohistochemistry techniques. As indicated in Table 3 with an example shown in FIG. 8, 1038_D5 (TCN-445) specifically bound to 6 out of 10 clinical isolates of an ovarian carcinoma, but it did not bind to any of 3 clinical isolates of normal ovary. Additionally 1038_D5 (TCN-445) did not bind other normal tissue such as brain heart, kidney, liver, lung, pancreas, muscle or spleen. This data demonstrates the tumor specificity of the antibody 1038_D5 (TCN-445), a highly desirable characteristic in a cancer therapeutic.

TABLE 3

| | # Samples Staining |
|---|---|
| Tissue | Positive |
| OVCAR3 | >75% |
| Ovarian Carcinoma | 6/10 |
| Normal Ovary | 0/3 |
| Brain, Cortex | 0/1 |
| Heart | 0/1 |
| Kidney | *0/1 |
| Liver | 0/1 |
| Lung | 0/1 |
| Pancreas | **1/1 |
| Skeletal Muscle | 0/1 |
| Spleen | ***0/1 |

*Background staining in PT similar to that obtained in absence of primary Ab
**Rare, inflammatory cells positive
***Granulocytes positive

### Example 6

### 1038_D5 (TCN-445) Tumor Cell Line Binding Survey

Cancer cells were contacted with TCN-445 to determine the ability of this monoclonal antibody to broadly bind to different subtypes of cancer. The results shown in Table 4 below demonstrate that TCN-445 binds to soft tissue sarcoma cells (A-673), pulmonary carcinoma cells (Calu-6), gastric carcinoma cells (Hs 746T), breast carcinoma cells (MCF-7), ovarian carcinoma cells (A2780 and OVCAR-3), and brain carcinoma (glioblastoma multiform) (U87 MG).

TABLE 4

| | | Relative |
|---|---|---|
| | | Staining |
| Cell Line | Type | Intensity |
| A-673 | Rhabdomyosarcoma | +++ |
| ARH-77 | Myeloma | − |
| Calu-6 | Pulmonary | + |
| FaDu | Squamous cell | − |
| Hs 746T | Gastric | ++ |
| HCT 116 | Colorectal | − |
| HT-29 | Colorectal | − |
| LoVo | Colorectal | − |
| LS 174T | Colorectal | − |
| MCF-7 | Breast | +++ |
| MX-1 | Breast | − |
| A2780 | Ovarian | ++ |
| OVCAR-3 | Ovarian | +++ |
| SU-DHL-4 | Follicular B cell | − |
| | lymphoma | |
| U-87 MG | Glioblastoma | + |
| | (multiform) | |

### Example 7

### Immunohistochemistry on Frozen Normal Tissues

### Summary

Antibody 1038_D5 (TCN-445) is a fully human monoclonal antibody targeting a protein expressed in cancer cells. The purpose of this study was to evaluate immunohistochemical staining of this antibody and the isotype control antibody 2N9 on frozen sections of human normal tissues, positive and negative control cell lines, and a tumor microarray of breast carcinoma samples.

For this portion of the study, immunohistochemistry was performed on a frozen normal tissue array, three full section samples of normal colon, three full section samples of normal small intestine, the positive cell line OVCAR3, and the negative cell line SKBR3. In addition, serial sections were evaluated with the isotype control antibody 2N9, as well as performing all procedures in the absence of primary antibody (including treatment with the anti-FITC antibody and HRP-DAB signal) as a control for background produced by the secondary antibody and detection system. The secondary antibody and detection system showed positive staining of granulocytes, macrophages, and rare fibroblasts but all other cell types in the tissues were negative.

The positive control cell line, OVCAR3, showed moderate to strong cytoplasmic and membranous staining of >75% of the cells with antibody 1038_D5 (TCN-445) at an antibody dilution of 1:100. The negative control cell line was negative with antibody 1038_D5 (TCN-445), and the isotype control antibody was negative within both the positive and negative control cell lines.

The three samples of normal colon and three samples of normal small intestine showed no significant differences in staining between antibody 1038_D5 (TCN-445) and the isotype control antibody at the tested concentration (1:100). Background staining within mucin, granulocytes, macrophages and occasional fibroblasts were present with either antibody followed by the secondary antibody and detection system.

In the normal frozen human tissue array, the following tissues showed no significant difference in staining compared to the isotype control antibody, and can be interpreted as negative for staining with antibody 1038_D5 (TCN-445) at a dilution of 1:100: adrenal, brain cerebellum, brain cerebrum, pituitary, breast, colon, esophagus, heart, kidney, liver, lung, skeletal muscle, mesothelium (pericardium), nerve, ovary, pancreas, placenta, prostate, salivary gland, skin, small intestine, spleen, stomach, testis, thymus, thyroid, tonsil, uterus with endometrium, uterine cervix, and bone marrow. In addition, the isotype control showed positive staining of epidermis that was negative with the test antibody.

In summary, antibody 1038_D5 (TCN-445), at a dilution of 1:100, showed moderate to strong staining of the positive control cells, was negative in the negative control cell line, and showed no significant positive staining within the 30 normal frozen human tissues tested when compared to the isotype control antibody.

### Methods

Antibody Titration Protocol, Positive Control Study Results, and Experimental Protocol: Antibody titration experiments were conducted with antibody 1038_D5 (TCN-445) and isotype control antibody 2N9 (supplied by Theraclone, humanized monoclonal antibodies; FITC labeled) to establish dilutions that would result in minimal background and maximal detection of signal. Serial dilutions were performed at 1:50, 1:100, 1:200 and 1:400 on fresh frozen tissues supplied by LifeSpan and positive and negative control cell lines supplied by Theraclone and prepared by LifeSpan. The dilution of 1:100 was selected for the study. Antibodies 1038_D5 (TCN-445) and 2N9 were used as the primary antibodies, and the principal detection system consisted of a Sigma anti-FITC secondary made in mouse (catalog# F-5636), with a DAKO Envision peroxidase labeled polymer (DAKO catalog# K4001) and DAB plus (DAKO catalog# K3468) as the chromagen, which was used to produce a brown-colored deposit.

Tissues were also stained with positive control antibodies (CD31 and vimentin) to ensure that the tissue antigens were preserved and accessible for immunohistochemical analysis. Only tissues that were positive for CD31 and vimentin staining were selected for the remainder of the study. The negative controls consisted of performing the entire immunohistochemistry procedure on adjacent sections in the absence of primary antibodies.

Slides stained at a dilution of 1:100 were imaged with a DVC 1310C digital camera coupled to a Nikon microscope. Images were stored as TIFF files with Adobe Photoshop.

Pathology scoring for the tabular portion of the study was reported based on the intensity of the staining, determined by the following numeric scale:

0=negative
1=blush
2=faint
3=moderate
4=strong
(NI)=not included (meaning cell type was not present)

### Full Section Colon and Small Intestine Samples

Three samples of frozen normal colon and three samples of frozen normal small intestine were analyzed. Full reporting on each sample is provided below.

### Colon

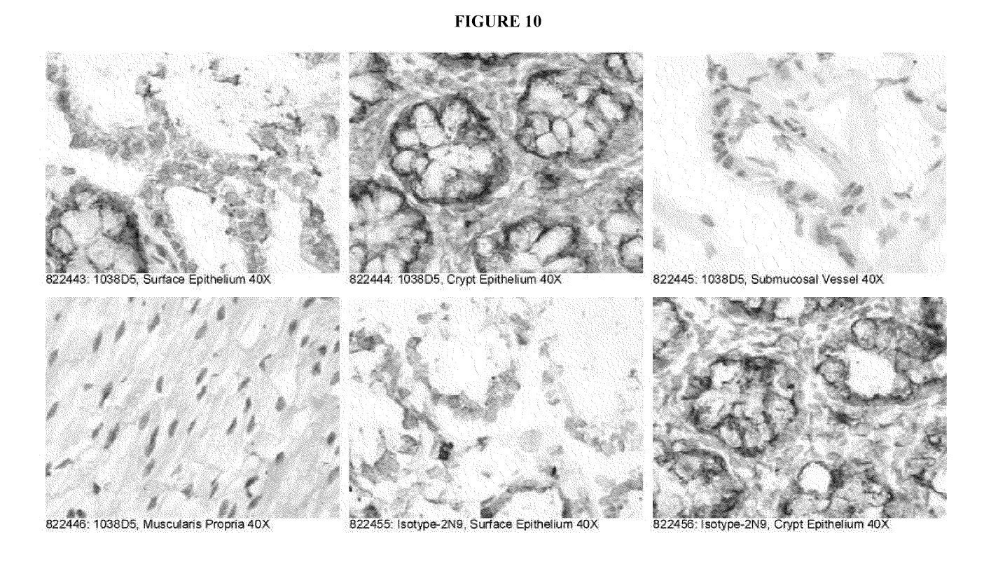

Sample 1: This sample of normal colon was obtained from a 41-year-old male who died of an intracerebral hemorrhage (FIG. 10).

Antibody 1038_D5 (TCN-445), at a dilution of 1:100, showed moderate membranous staining in absorptive epithelium that slightly exceeded the background level seen with the isotype control antibody. The level of staining differed very minimally between antibody 1038_D5 (TCN-445) and the isotype control antibody. Smooth muscle, vessels, and the myenteric plexus were negative. Granulocytes or macrophages showed positive staining that was identical to the background staining seen with the isotype control antibody.

The isotype control antibody showed faint to occasionally moderate staining of mucinous secretions along the luminal border and moderate staining of granulocytes or macrophages similar to the background seen with the secondary antibody and DAB detection system.

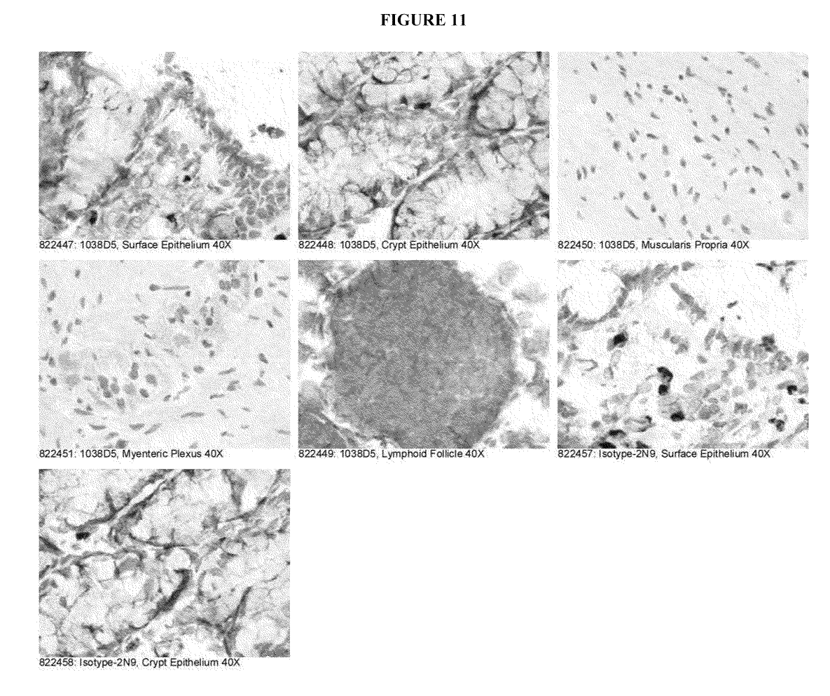

Sample 2: This sample of normal colon was obtained at transverse colon resection from a 76-year-old female (FIG. 11).

Antibody 1038_D5 (TCN-445), at a dilution of 1:100, showed faint to moderate membranous staining in absorptive epithelium that did not differ significantly from the background level seen with the isotype control antibody. Smooth muscle, vessels, and other stromal cells were negative.

Granulocytes or macrophages showed positive staining that was identical to the background staining seen with the isotype control antibody.

The isotype control antibody showed faint to moderate staining of mucinous secretions along the luminal border and moderate staining of granulocytes or macrophages similar to the background seen with the secondary antibody and DAB detection system.

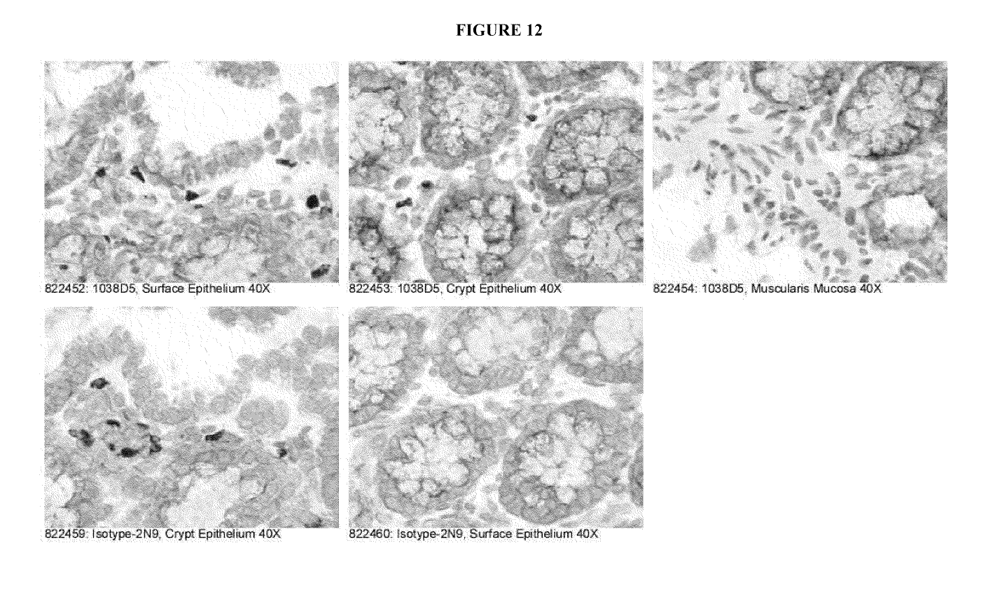

Sample 3: This sample of normal colon was obtained at sigmoid colon resection from a 59-year old female (FIG. 12).

Antibody 1038_D5 (TCN-445), at a dilution of 1:100, showed patchy faint to moderate membranous staining in absorptive epithelium that did not differ significantly from the background level seen with the isotype control antibody. Smooth muscle, vessels, and lymphocytes were negative. Granulocytes or macrophages also showed positive staining that was identical to the background staining seen with the isotype control antibody.

The isotype control antibody showed faint to moderate staining of mucinous secretions along the luminal border and moderate staining of granulocytes or macrophages similar to the background seen with the secondary antibody and DAB detection system.

### Small Intestine

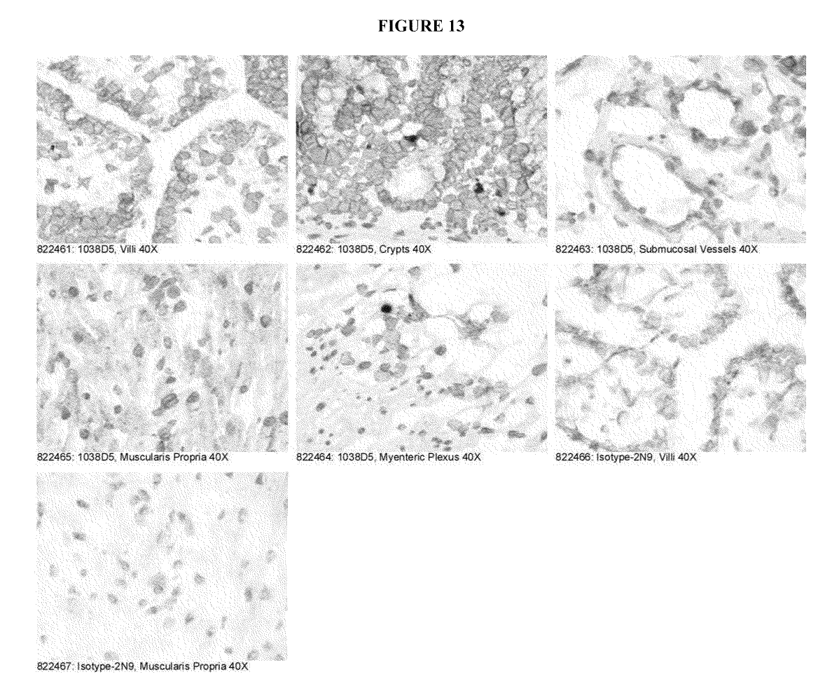

Sample 1: This sample of normal small intestine was obtained from a 56-year-old female (FIG. 13).

Antibody 1038_D5 (TCN-445), at a dilution of 1:100, showed patchy faint to moderate membranous staining in absorptive epithelium that did not differ significantly from the background level seen with the isotype control antibody. Lymphocytes were negative, as were plasma cells and vessels. Smooth muscle of the muscularis mucosa and muscularis propria showed patchy faint staining.

Granulocytes or macrophages showed positive staining that was identical to the background staining seen with the isotype control antibody.

The isotype control antibody showed faint to moderate staining of mucinous secretions along the luminal border and moderate staining of granulocytes or macrophages similar to the background seen with the secondary antibody and DAB detection system.

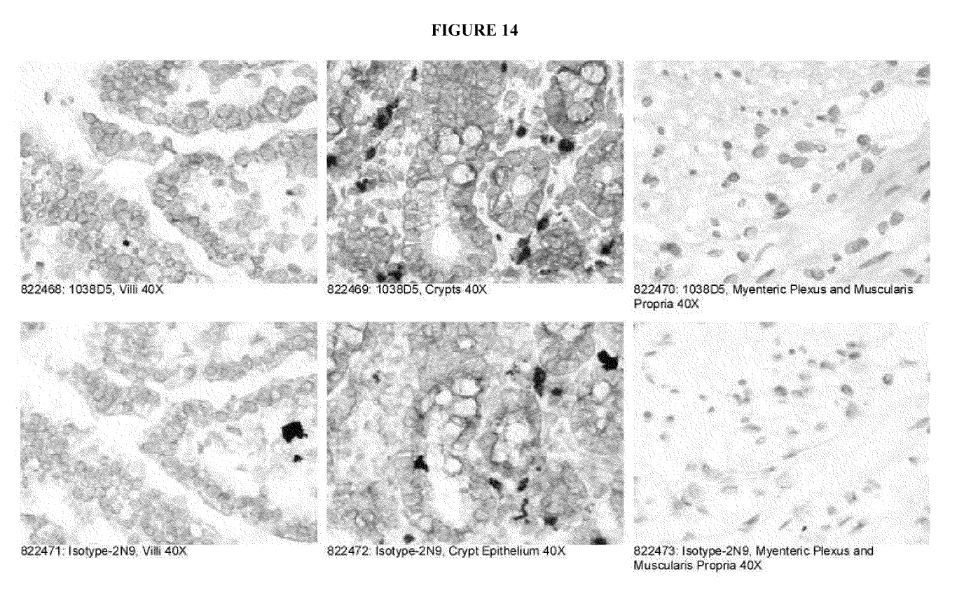

Sample 2: This sample of normal small intestine was obtained from a 51-year-old female (FIG. 14).

Antibody 1038_D5 (TCN-445), at a dilution of 1:100, showed faint to moderate membranous staining in absorptive epithelium that did not differ significantly from the background level seen with the isotype control antibody. Lymphocytes were negative, as were plasma cells and vessels. Smooth muscle of the muscularis mucosa and muscularis propria showed negative to occasional patchy faint staining. Granulocytes or macrophages showed positive staining that was identical to the background staining seen with the isotype control antibody.

The isotype control antibody showed faint to moderate staining of mucinous secretions along the luminal border and moderate staining of granulocytes or macrophages similar to the background seen with the secondary antibody and DAB detection system.

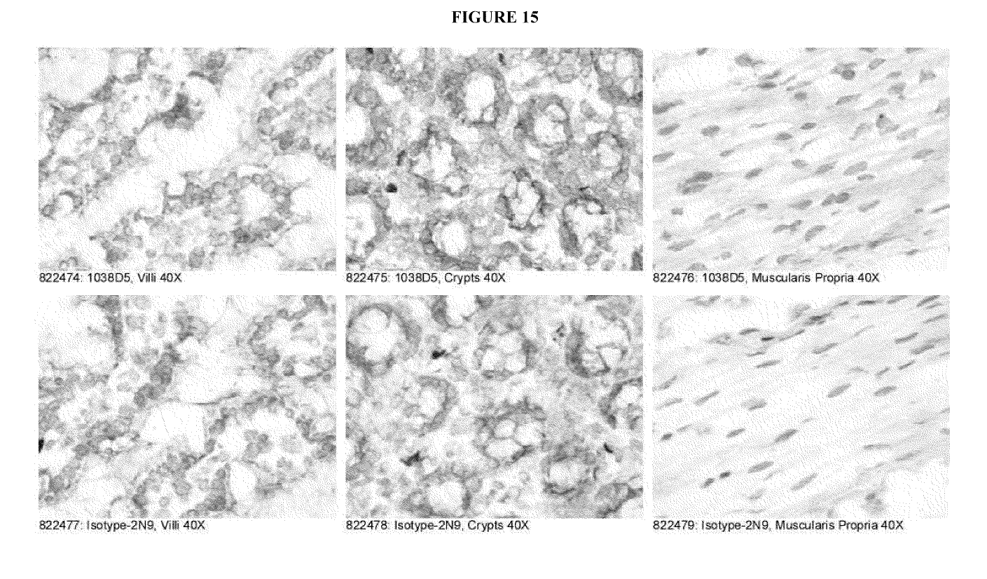

Sample 3: This sample of normal small intestine was obtained from a 55-year-old male who died of an intracranial hemorrhage (FIG. 15).

Antibody 1038_D5 (TCN-445), at a dilution of 1:100, showed largely faint to rare moderate membranous staining in absorptive epithelium that did not differ significantly from the background level seen with the isotype control antibody. Plasma cells, fibroblasts and vessels were largely negative or showed blush background staining. Smooth muscle of the muscularis mucosa and muscularis propria showed negative to occasional patchy faint staining. Granulocytes or macrophages showed positive staining that was identical to the background staining seen with the isotype control antibody.

The isotype control antibody showed faint to moderate staining of mucinous secretions along the luminal border and moderate staining of granulocytes or macrophages similar to the background seen with the secondary antibody and DAB detection system.

TABLE 5
Tabular Data for Full Sections of Normal Colon

| | |
|---|---|
| 1038D5 | Isotype-2N9 |

Sample 1

| | | | | | | |
|---|---|---|---|---|---|---|
| Epithelium | 3-4 | M | Many | 3-4 | M | Many |
| Sm muscle | 0 | | | 0 | | |
| Inf cell | 2-3 | C | | 2-3 | C | |
| Gang cell | 0 | | | 0 | | |

Sample 2

| | | | | | | |
|---|---|---|---|---|---|---|
| Epithelium | 2-3 | M | Many | 2-3 | M | Many |
| Sm muscle | 0 | | | 0 | | |
| Inf cell | 3 | C | Occ | 3 | C | Occ |
| Gang cell | 0 | | | 0 | | |

Sample 3

| | | | | | | |
|---|---|---|---|---|---|---|
| Epithelium | 2-3 | M | | 2-3 | M | |
| Sm muscle | 0 | | | 0 | | |
| Inf cell | 3-4 | C | Occ | 3-4 | C | Occ |
| Gang cell | 0 | | | 0 | | |

| Cells | Type | |
|---|---|---|
| OVCAR3 | Positive | 3-4 |
| SKBR3 | Negative | 0 |

TABLE 6
Tabular Data for Full Sections of Normal Small Intestine

| | |
|---|---|
| 1038D5 | Isotype-2N9 |

Sample 1

| | | | | | | |
|---|---|---|---|---|---|---|
| Epithelium | 2-3 | M | Occ | 2-3 | M | Occ |
| Sm muscle | 2 | C | Occ | 0 | | |
| Inf cell | 3-4 | C | Occ | 3-4 | C | Occ |
| Gang cell | 0 | | | 0 | | |

Sample 2

| | | | | | | |
|---|---|---|---|---|---|---|
| Epithelium | 2-3 | M | Many | 2-3 | M | Many |
| Sm muscle | 2 | C | Occ | 0 | | |
| Inf cell | 3-4 | C | Occ | 3-4 | C | Occ |
| Gang cell | 0 | | | 0 | | |

Sample 3

| | | | | | | |
|---|---|---|---|---|---|---|
| Epithelium | 2-3 | M | Many | 2-3 | M | Many |
| Sm muscle | 2 | C | Occ | 0 | | |
| Inf cell | 2-3 | C | Occ | 2-3 | C | Occ |
| Gang cell | 0 | | | 0 | | |

Key:
Sm muscle: smooth muscle
Inf cell: inflammatory cell
Gang cell: ganglion cell
Occ: occasional

### Example 8

### FDA Tissue Microarray

### Donor Information

Donor tissue is human. Moreover the tissue is derived from normal adults. Normal, as used in this example, is defined as not containing a tumor. Alternatively, or in addition, normal tissue is harvested from a human who has not been diagnosed as having any known disease. Moreover, normal tissue may be derived from a donor who died from a disease that could not have affected the donated organ, allowing the donated organ to be considered normal.

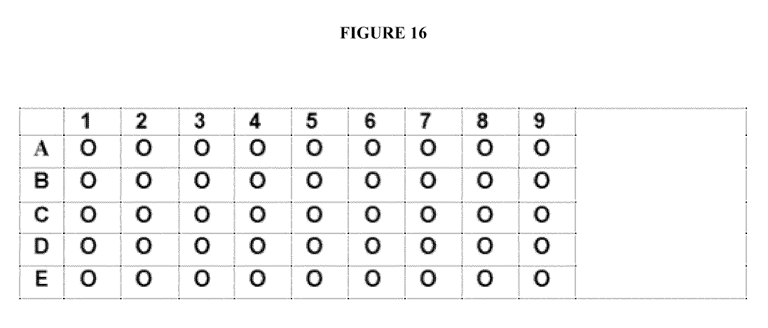

TABLE 7
Slide 1 (see FIG. 16 for microarray schematic)

| Position | Tissue Type | Age | Gender | Diagnosis |
|---|---|---|---|---|
| A1 | Adrenal 1 | 80 | M | Normal |
| A2 | Adrenal 2 | 70 | F | Normal |
| A3 | Adrenal 3 | 63 | M | Normal |
| A4 | Brain, Cerebellum 1 | 70 | F | Normal |
| A5 | Brain, Cerebellum 2 | 84 | M | Normal |
| A6 | Brain, Cerebellum 3 | 60 | F | Normal |
| A7 | Brain, Cerebrum 1 | 70 | F | Normal |
| A8 | Brain, Cerebrum 2 | 60 | M | Normal |
| A9 | Brain, Cerebrum 3 | 84 | M | Normal |
| B1 | Brain, pituitary 1 | 76 | F | Normal |
| B2 | Brain, pituitary 2 | 63 | M | Normal |
| B3 | Brain, Pituitary 3 | 57 | F | Normal |
| B4 | Breast 1 | 41 | F | Normal |
| B5 | Breast 2 | 56 | F | Normal |
| B6 | Breast 3 | 46 | F | Normal |
| B7 | Colon 1 | 56 | M | Normal |
| B8 | Colon 2 | 26 | M | Normal |
| B9 | Colon 3 | 17 | M | Normal |
| C1 | Esophagus 1 | 21 | F | Normal |
| C2 | Esophagus 2 | 66 | M | Normal |
| C3 | Esophagus 3 | 79 | M | Normal |
| C4 | Heart 1 | 70 | F | Normal |
| C5 | Heart 2 | 87 | F | Normal |
| C6 | Heart 3 | 57 | F | Normal |
| C7 | Kidney 1 | 74 | F | Normal |
| C8 | Kidney 2 | 63 | F | Normal |
| C9 | Kidney 3 | 62 | M | Normal |
| D1 | Liver 1 | 70 | F | Normal |
| D2 | Liver 2 | 71 | M | Normal |
| D4 | Liver 3 | 30 | M | Normal |
| D4 | Lung 1 | 57 | M | Normal |
| D5 | Lung 2 | 26 | M | Normal |
| D6 | Lung 3 | 83 | F | Normal |
| D7 | Skeletal Muscle 1 | 71 | M | Normal |
| D8 | Skeletal Muscle 2 | 79 | M | Normal |
| D9 | Skeletal muscle 3 | 57 | F | Normal |
| E1 | Mesothileal 1 | 70 | F | Normal, pericardium |
| E2 | Mesothileal 2 | 28 | M | Normal, pericardium |
| E3 | Mesothileal 3 | 82 | F | Normal, pericardium |
| E4 | Nerve 1 | 66 | M | Normal |
| E5 | Nerve 2 | 84 | F | Normal |
| E6 | Nerve 3 | 78 | F | Normal |
| E7 | Ovary 1 | 78 | F | Normal |
| E8 | Ovary 2 | 37 | F | Normal |
| E9 | Ovary 3 | 74 | F | Normal |

TABLE 8
Slide 2 (see FIG. 16 for arrangement)

| Position | Tissue Type | Age | Gender | Diagnosis |
|---|---|---|---|---|
| A1 | Pancreas 1 | 62 | M | Normal |
| A2 | Pancreas 2 | 69 | F | Normal |
| A3 | Pancreas 3 | 44 | M | Normal |
| A4 | Placenta 1 | 25 | F | Normal |
| A5 | Placenta 2 | 29 | F | Normal |
| A6 | Placenta 3 | 27 | F | Normal |
| A7 | Prostate 1 | 44 | M | Normal |
| A8 | Prostate 2 | 50 | M | Normal |
| A9 | Prostate 3 | 69 | M | Normal |
| B1 | Salivary Gland 1 | 66 | M | Normal |
| B2 | Salivary gland 2 | 44 | M | Normal |
| B3 | Salivary Gland 3 | 84 | M | Normal |
| B4 | Skin 1 | 83 | F | Normal |
| B5 | Skin 2 | 44 | F | Normal |
| B6 | Skin 3 | 21 | F | Normal |
| B7 | Small intestine 1 | 86 | F | Normal |
| B8 | Small intestine 2 | 50 | M | Normal |
| B9 | Small Intestine 3 | 50 | F | Normal |
| C1 | Spleen 1 | 24 | M | Normal |
| C2 | Spleen 2 | 70 | F | Normal |
| C3 | Spleen 3 | 26 | M | Normal |
| C4 | Stomach 1 | 71 | M | Normal |
| C5 | Stomach 2 | 56 | M | Normal |
| C6 | Stomach 3 | 24 | M | Normal |
| C7 | Testis 1 | 23 | M | Normal |
| C8 | Testis 2 | 26 | M | Normal |
| C9 | Testis 3 | 26 | M | Normal |
| D1 | Thymus 1 | 24 | M | Normal |
| D2 | Thymus 2 | 28 | M | Normal |
| D3 | Thymus 3 | 28 | M | Normal |
| D4 | Thyroid 1 | 62 | M | Normal |
| D5 | Thyroid 2 | 25 | M | Normal |
| D6 | Thyroid 3 | 59 | M | Normal |
| D7 | Tonsil 1 | 15 | F | Normal |
| D8 | Tonsil 2 | 25 | M | Normal |
| D9 | Tonsil 3 | 20 | F | Normal |
| E1 | Uterus 1 | 56 | F | Normal |
| E2 | Uterus 2 | 47 | F | Normal |
| E3 | Uterus 3 | 32 | F | Normal |
| E4 | Uterus, Cervix 1 | 42 | F | Normal |
| E5 | Uterus, Cervix 2 | 49 | F | Normal |
| E6 | Uterus, Cervix 3 | 30 | F | Normal |
| E7 | Bone Marrow 1 | 86 | F | Normal |
| E8 | Bone marrow 2 | 70 | M | Normal |
| E9 | Bone Marrow 3 | 61 | F | Normal |

### Tabular Data for Normal Frozen Samples on FDA Tissue Microarray

Tables 9 and 11, corresponding to slides 1 and 2, respectively, demonstrate that the 1038_D5 (TCN-445) antibody provides minimal staining for normal, non-cancerous tissue. These data are similar to the results observed for the isotype control antibody, 2N9, which is specific for cytomegalovirus (CMV) (Tables 10 and 12, corresponding to slides 1 and 2, respectively).

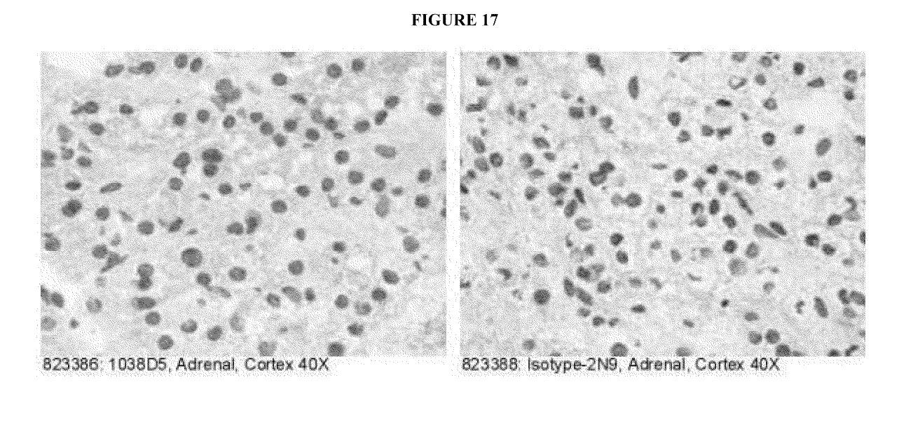

Representative photographs that depict staining of both the monoclonal antibody 1038_D5 (TCN-445) and the isotype control antibody 2N9 are shown in FIGS. 17-23.

Representative Images from Tissues on the Frozen FDA Array

Adrenal (Slide 1, Position A2): This sample of normal adrenal gland was obtained from a 70-year-old female (FIG. 17).

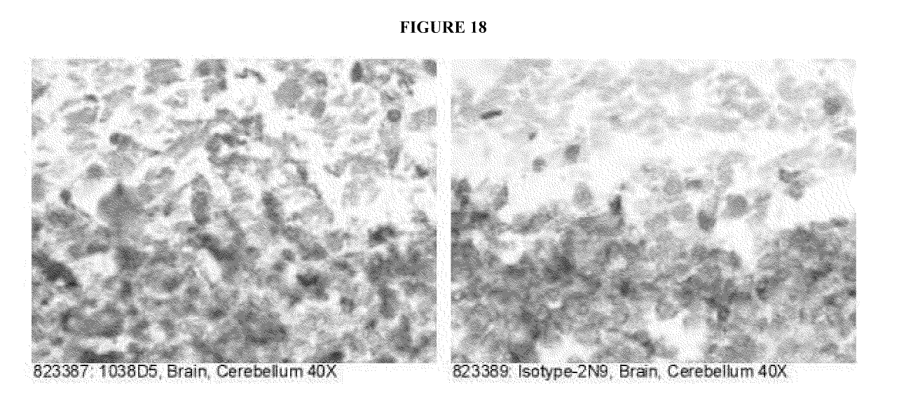

Brain, Cerebellum (Slide 1, Position A6): This sample of normal brain was obtained from a 60-year-old female (FIG. 18).

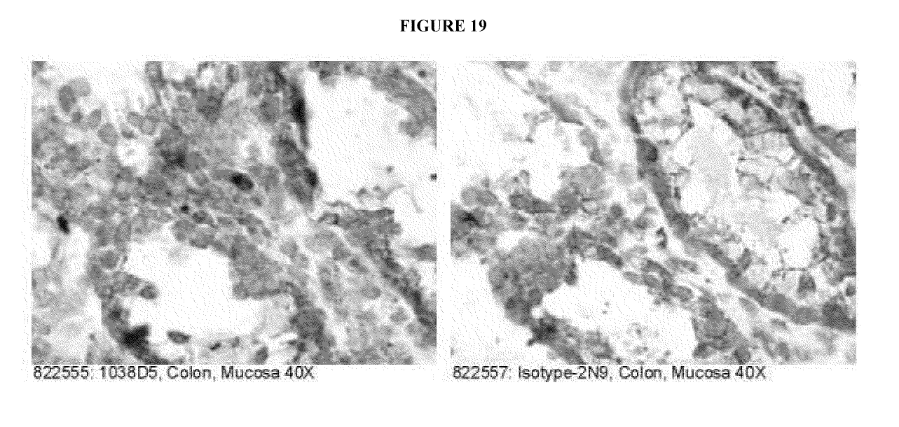

Colon (Slide 1, Position B7): This sample of normal colon was obtained from a 56-year-old male (FIG. 19).

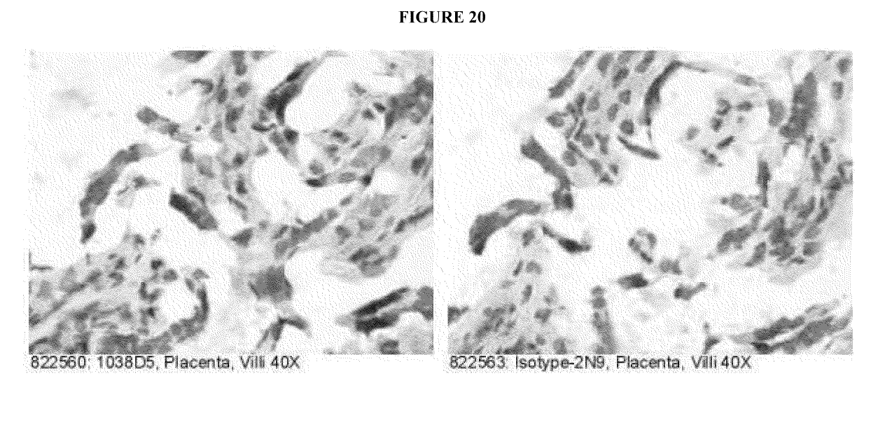

Placenta (Slide 2, Position A5): This sample of normal placenta was obtained from a 29-year-old female (FIG. 20).

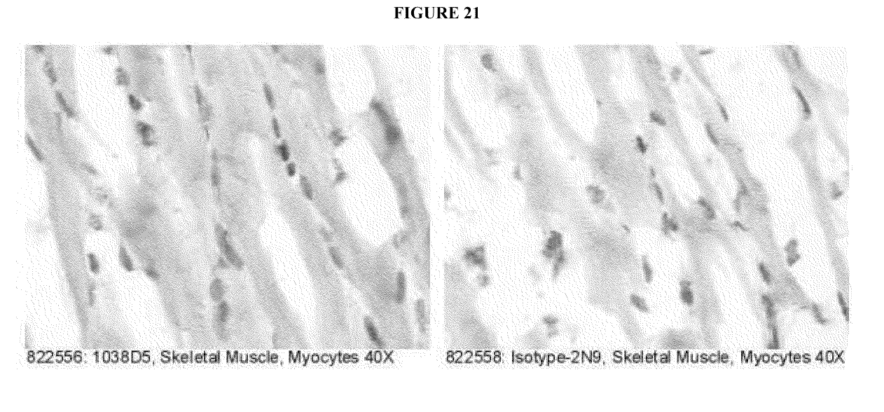

Skeletal Muscle (Slide 1, Position D7): This sample of normal skeletal muscle was obtained from a 71-year-old male (FIG. 21).

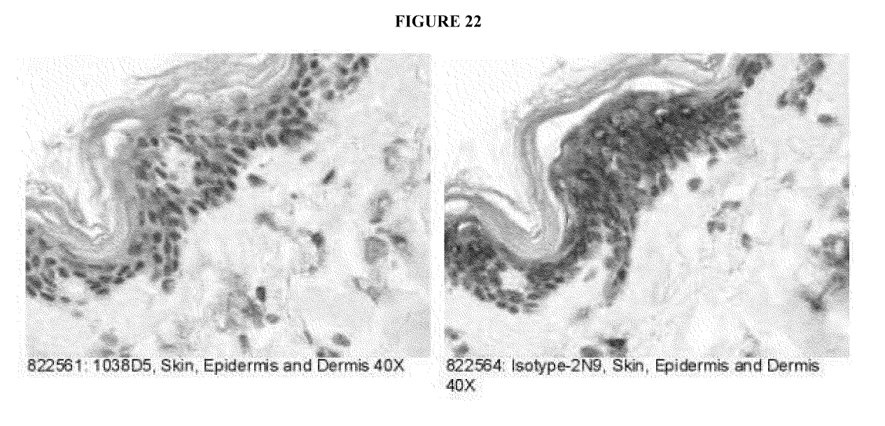

Skin (Slide 2, Position B4): This sample of normal skin was obtained from an 83-year-old female (FIG. 22).

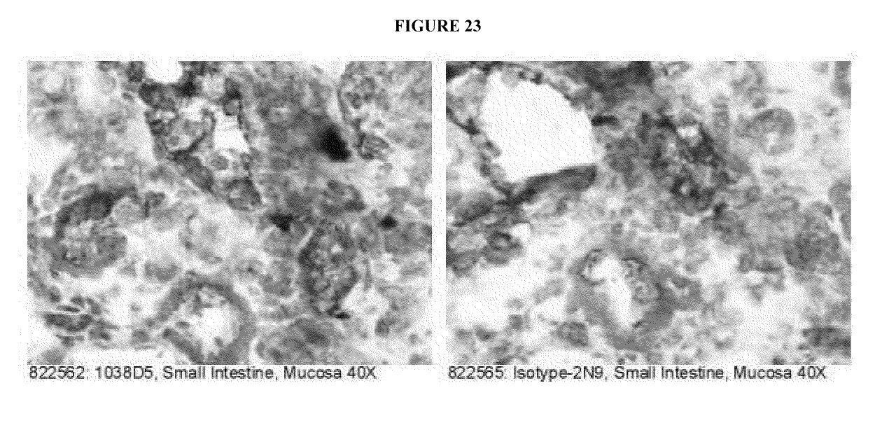

Small Intestine (Slide 2, Position B8): This sample of normal small intestine was obtained from a 50-year-old male (FIG. 23).

Accordingly, the staining present in these photographs is minimal, because the tissue is normal, non-cancerous tissue.

These data and photographs recapitulate the expected results for the application of cancer and CMV specific antibodies to non-cancerous and non-infected tissue. Consequently, a positive result obtained with an anti-cancer antibody of the invention would be readily discernable over the minimal background staining described herein.

TABLE 9
Slide 1 - 1038_D5 (TCN-445)

| | 1 | | | 2 | | | 3 | | | 4 | | | 5 | | |
|---|---|---|---|---|---|---|---|---|---|---|---|---|---|---|---|
| | Adrenal | | | Adrenal | | | Adrenal | | | Brain, Cerebellum | | | Brain, Cerebellum | | |
| A | cx | 0 | | cx | 0 | | cx | 0 | | neu | 0 | | neu | 0 | |
| | med | NA | | med | NA | | med | NA | | glia | 0 | | glia | 0 | |
| | other | 0 | | other | 0 | | other | 0 | | other | 0 | | other | 0 | |
| | Brain, pituitary | | | Brain, pituitary | | | Brain, pituitary | | | Breast | | | Breast | | |
| B | anterior | 0 | | anterior | 0-2 | rare | anterior | 0-2 | rare | epi | 0-2 | rare | epi | 0-2 | rare |
| | | | | | [C] | | | [C] | | | [C] | | | [C] | |
| | posterior | NA | | posterior | NA | | posterior | NA | | stroma | 0 | | stroma | 0 | |
| | other | 2 | occ | other | 2 | rare | other | 2 | rare | other | 0 | | other | 0 | |
| | | [C] | | | [C] | | | [C] | | | | | | | |
| | esophagus | | | esophagus | | | esophagus | | | Heart | | | Heart | | |
| C | epi | 0 | | epi | 0 | | epi | NA | | myo | 0 | | myo | 0 | |
| | sm musc | 0 | | sm musc | 0 | | sm musc | 0 | | other | 0 | | Other- | 2 | rare |
| | | | | | | | | | | | | | fib | [C] | |
| | Skel musc | NA | | Skel musc | NA | | Skel musc | NA | | | | | | | |
| | other | 0 | | Other- | 3 | | Other- | 3 | | | | | | | |
| | | | | inf | [C] | | inf | [C] | | | | | | | |
| | Liver | | | Liver | | | Liver | | | Lung | | | Lung | | |
| D | hep | 0 | | hep | 0 | | hep | 0-1 | | airway | NA | | airway | NA | |
| | | | | | | | | [C\| | | | | | | | |
| | bile duct | 0 | | bile duct | 0 | | bile duct | 0 | | pneum | 0 | | pneum | 0 | |
| | kupf | 2-3 | | kupf | 2 | | kupf | 0-1 | | al mac | 0 | | al mac | 0-1 | |
| | | [C] | | | [C] | | | [C] | | | | | | [C] | |
| | other | 0 | | other | 0 | | other | 0 | | other | 0 | | Other- | 3 | |
| | | | | | | | | | | | | | gran | [C] | |
| | Mesothelium | | | Mesothelium | | | Mesothelium | | | Nerve | | | Nerve | | |
| E | meso | 0 | | meso | NA | | meso | NA | | Schwann | 0 | | Schwann | 0 | |
| | stroma | NA | | stroma | 0 | | stroma | 0 | | other | 0 | | other | 0 | |
| | Other- | 2 | | other | 0 | | other | 0 | | | | | | | |
| | mac. fib | [C] | | | | | | | | | | | | | |

| | 6 | | | 7 | | 8 | | 9 | | |
|---|---|---|---|---|---|---|---|---|---|---|
| | Brain, Cerebellum | | | Brain, Cerebellum | | Brain, Cerebellum | | Brain, Cerebellum | | |
| A | neu | 0 | | neu | 0 | neu | 0 | neu | 0 | |
| | glia | 0 | | glia | 0 | glia | 0 | glia | 0 | |
| | other | 0 | | other | 0 | other | 0 | other | 0 | |
| | Breast | | | Colon | | Colon | | Colon | | |
| B | epi | 0-2 | rare | epi | 3 | epi | 3 | epi | 3 | |
| | | [C] | | | [C, M] | | [C, M] | | [C, M] | |
| | stroma | 0 | | sm | 1-2 | sm | 0 | sm | 0 | |
| | | | | | [C] | | | | | |
| | other | 0 | | inf | 3 | inf | 3 | inf | 3 | |
| | | | | | [C] | | [C] | | [C] | |
| | | | | gang | NA | gang | NA | gang | NA | |
| | | | | other | 0 | other | 0 | other | 0 | |
| | Heart | | | Kid cx | | Kid cx | | Kid cx | | |
| C | myo | 0 | | Glom | 0 | Glom | 0 | Glom | 0 | |
| | Other | 0 | | RTE | 0 | RTE | 0 | RTE | 0-2 | rare |
| | | | | | | | | | [C] | |
| | | | | other | 0 | other | 0 | other | 0 | |
| | Lung | | | Skeletal Muscle | | Skeletal Muscle | | Skeletal Muscle | | |
| D | airway | NA | | myo | 0 | myo | 0 | myo | 0 | |
| | pneum | 0 | | other | 0 | other | 0 | other | 0 | |
| | al mac | 0 | | | | | | | | |
| | Other- | 3 | | | | | | | | |
| | gran | [C] | | | | | | | | |
| | Nerve | | | Ovary | | Ovary | | Ovary | | |
| E | Schwann | 0 | | follicle | 0 | follicle | NA | follicle | NA | |
| | other | 0 | | stroma | 0 | stroma | 0 | stroma | 0 | |
| | | | | vessels | 0 | vessels | 0 | vessels | 0 | |
| | | | | other | 0 | other | 0 | other | 0 | |

TABLE 10
Slide 1 - Isotype 2N9

| | 1 | | | 2 | | | 3 | | | 4 | | | 5 | | |
|---|---|---|---|---|---|---|---|---|---|---|---|---|---|---|---|
| | Adrenal | | | Adrenal | | | Adrenal | | | Brain, Cerebellum | | | Brain, Cerebellum | | |
| A | cx | 0 | | cx | 0 | | cx | 0 | | neu | 0 | | neu | 0 | |
| | med | NA | | med | NA | | med | NA | | glia | 0 | | glia | 0 | |
| | other | 0 | | other | 0 | | other | 0 | | other | 0 | | other | 0 | |
| | Brain, pituitary | | | Brain, pituitary | | | Brain, pituitary | | | Breast | | | Breast | | |
| B | anterior | 0 | | anterior | 0-2 | rare | anterior | 0-2 | rare | epi | 0-2 | rare | epi | 0-2 | rare |
| | | | | | [C] | | | [C] | | | [C] | | | [C] | |
| | posterior | NA | | posterior | NA | | posterior | NA | | stroma | 0 | | stroma | 0 | |
| | other | 2 | rare | other | 2 | rare | other | 2 | rare | other | 0 | | other | 0 | |
| | | [C] | | | [C] | | | [C] | | | | | | | |
| | esophagus | | | esophagus | | | esophagus | | | Heart | | | Heart | | |
| C | epi | 0 | | epi | 0 | | epi | NA | | myo | 0 | | myo | 0 | |
| | sm musc | 0 | | sm musc | 0 | | sm musc | 0 | | other | 0 | | Other- | 0 | |
| | | | | | | | | | | | | | fib | | |
| | Skel musc | NA | | Skel musc | NA | | Skel musc | NA | | | | | | | |
| | other | 0 | | Other- | 3 | | Other- | 3 | | | | | | | |
| | | | | inf | [C] | | inf | [C] | | | | | | | |
| | Liver | | | Liver | | | Liver | | | Lung | | | Lung | | |
| D | hep | 0 | | hep | 0 | | hep | 0 | | airway | NA | | airway | NA | |
| | bile duct | 0 | | bile duct | 0 | | bile duct | 0 | | pneum | 0 | | pneum | 0 | |
| | kupf | 2-3 | | kupf | 2 | | kupf | 0-1 | | al mac | 0 | | al mac | 0-1 | |
| | | [C] | | | [C] | | | [C] | | | | | | [C] | |
| | other | 0 | | other | 0 | | other | 0 | | other | 0 | | Other- | 3 | |
| | | | | | | | | | | | | | gran | [C] | |
| | Mesothelium | | | Mesothelium | | | Mesothelium | | | Nerve | | | Nerve | | |
| E | meso | 0 | | meso | NA | | meso | NA | | Schwann | 0 | | Schwann | 0 | |
| | stroma | NA | | stroma | 0 | | stroma | 0 | | other | 0 | | other | 0 | |
| | Other- | 2 | | other | 0 | | other | 0 | | | | | | | |
| | mac, fib | [C] | | | | | | | | | | | | | |

| | 6 | | | 7 | | 8 | | 9 | | |
|---|---|---|---|---|---|---|---|---|---|---|
| | Brain, Cerebellum | | | Brain, Cerebellum | | Brain, Cerebellum | | Brain, Cerebellum | | |
| A | neu | 0 | | neu | 0 | neu | 0 | neu | 0 | |
| | glia | 0 | | glia | 0 | glia | 0 | glia | 0 | |
| | other | 0 | | other | 0 | other | 0 | other | 0 | |
| | Breast | | | Colon | | Colon | | Colon | | |
| B | epi | 0-2 | rare | epi | 3 | epi | 3 | epi | 3 | |
| | | [C] | | | [C, M] | | [C, M] | | [C, M] | |
| | stroma | 0 | | sm | 1-2 | sm | 0 | sm | 0 | |
| | other | 0 | | inf | 3 | inf | 3 | inf | 3 | |
| | | | | | [C] | | [C] | | [C] | |
| | | | | gang | NA | gang | NA | gang | NA | |
| | | | | other | 0 | other | 0 | other | 0 | |
| | Heart | | | Kid cx | | Kid cx | | Kid cx | | |
| C | myo | 0 | | Glom | 0 | Glom | 0 | Glom | 0 | |
| | Other | 0 | | RTE | 0 | RTE | 0 | RTE | 0-2 | rare |
| | | | | | | | | | [C] | |
| | | | | other | 0 | other | 0 | other | 0 | |
| | Lung | | | Skeletal Muscle | | Skeletal Muscle | | Skeletal Muscle | | |
| D | airway | NA | | myo | 0 | myo | 0 | myo | 0 | |
| | pneum | 0 | | other | 0 | other | 0 | other | 0 | |
| | al mac | 0 | | | | | | | | |
| | Other- | 3 | | | | | | | | |
| | gran | [C] | | | | | | | | |
| | Nerve | | | Ovary | | Ovary | | Ovary | | |
| E | Schwann | 0 | | follicle | NA | follicle | NA | follicle | NA | |
| | other | 0 | | stroma | 0 | stroma | 0 | stroma | 0 | |
| | | | | vessels | 0 | vessels | 0 | vessels | 0 | |
| | | | | other | 0 | other | 0 | other | 0 | |

TABLE 11
Slide 2 - 1038_D5 (TCN-445)

| | 1 | | 2 | | | 3 | | 4 | | | 5 | | |
|---|---|---|---|---|---|---|---|---|---|---|---|---|---|
| | Pancreas | | Pancreas | | | Pancreas | | Placenta | | | Placenta | | |
| A | acini | 0-1 | acini | 0-1 | | acini | 2-3 | tropho | 0 | | tropho | 0 | |
| | | [C] | | [C] | | | [C] | | | | | | |
| | islet | 0 | islet | 0 | | islet | 0 | stroma | 3 | | stroma | 2 | |
| | | | | | | | | | [C, M] | | | [C] | |
| | duct | 0 | duct | 0 | | duct | 0 | other | 0 | | other | 0 | |
| | other - | 0-1 | other - | 0 | | other - | 2 | | | | | | |
| | mac, fib | [C] | mac, fib | | | mac, fib | [C] | | | | | | |
| | Salivary Gland | | Salivary Gland | | | Salivary Gland | | Skin | | | Skin | | |
| B | epi | 0 | epi | 0 | | epi | 0 | epiderm | 0 | | epiderm | 0 | |
| | other | 0 | other | 2 | rare | other | 0 | derm | 2 | rare | derm | 2 | rare |
| | | | | [C] | | | | (fib) | [C] | | (fib) | [C] | |
| | | | | | | | | other | 0 | | other | 0 | |
| | Spleen | | Spleen | | | Spleen | | Stomach | | | Stomach | | |
| C | lym | 0 | lym | 0 | | lym | 0 | epi | NA | | epi | 2 | SS |
| | | | | | | | | | | | | [C] | |
| | rd pulp | 0 | rd pulp | 0 | | rd pulp | 0 | sm | 0 | | sm | 0 | |
| | other- | 3 | other- | 3 | | other- | 3 | other | 0 | | other | 2 | rare |
| | gran | [C] | gran | [C] | | gran | [C] | | | | | [C] | |
| | Thymus | | Thymus | | | Thymus | | Thyroid | | | Thyroid | | |
| D | lym | 0 | lym | 0 | | lym | 0 | follicle | 0 | | follicle | 0 | |
| | epi | 0 | epi | 0 | | epi | 0 | other - | 3 | rare | other - | 0 | |
| | | | | | | | | fib, gran | [C] | | fib, gran | | |
| | other - | 3 | other - | 3 | | other - | 3 | | | | | | |
| | gran | [C] | gran | [C] | | gran | [C] | | | | | | |
| | Uterus | | Uterus | | | Uterus | | Uterus, Cervix | | | Uterus, Cervix | | |
| E | endo | NA | endo | 0 | | endo | 0 | epi | NA | | epi | NA | |
| | myo | 0 | myo | 0 | | myo | 0 | gland | NA | | gland | NA | |
| | other - | 3 | other - | 0 | | other - | 0 | sm | 0 | | sm | 0 | |
| | gran | [C] | gran | | | gran | | | | | | | |
| | | | | | | | | other | 0 | | other | 0 | |

| | 6 | | 7 | | 8 | | 9 | |
|---|---|---|---|---|---|---|---|---|
| | Placenta | | Prostate | | Prostate | | Prostate | |
| A | tropho | 0 | gland | 0 | gland | 0 | gland | 0 |
| | stroma | 2-3 | stroma | 2 | stroma | 0-3 | stroma | 0-3 |
| | | [C, M] | | [C] | | [C] | | [C] |
| | other | 0 | other | 0 | other | 0 | other | 0 |

| | | | |
|---|---|---|---|
| Skin | Small Intestine | Small Intestine | Small Intestine |

| | | | | | | | | | | | | |
|---|---|---|---|---|---|---|---|---|---|---|---|---|
| B | epiderm | 0 | | epi | 3 | | epi | 3 | | epi | 3 | |
| | | | | | [C, M] | | | [C, M] | | | [C, M] | |
| | derm | 2 | rare | sm | 0-1 | | sm | 0 | | sm | 0 | |
| | (fib) | [C] | | | [C] | | | | | | | |
| | other | 0 | | inf | 3 | rare | inf | 3 | rare | inf | 3 | rare |
| | | | | | [C] | | | [C] | | | [C] | |
| | | | | gang | 0 | | gang | NA | | gang | 0 | |
| | | | | other | 0 | | other | 0 | | other | 0 | |

| | | | |
|---|---|---|---|
| Stomach | Testis | Testis | Testis |

| | | | | | | | | | | |
|---|---|---|---|---|---|---|---|---|---|---|
| C | epi | NA | | semi | 0 | | semi | 0 | semi | 0 |
| | sm | 0 | | leydig | 2 | rare | leydig | 0 | leydig | 0 |
| | | | | | [C] | | | | | |
| | other | 2 | rare | other | 0 | | other | 0 | other | 0 |
| | | [C] | | | | | | | | |

| | | | |
|---|---|---|---|
| Thyroid | Tonsil | Tonsil | Tonsil |

| | | | | | | | | | | | |
|---|---|---|---|---|---|---|---|---|---|---|---|
| D | follicle | 0 | epi | 0 | | epi | 0 | | epi | 0 | |
| | other - | 0 | lym | 0 | | lym | 0 | | lym | 0 | |
| | fib, gran | | | | | | | | | | |
| | | | other - | 3 | rare | other - | 3 | rare | other - | 3 | rare |
| | | | gran | [C] | | gran | [C] | | gran | [C] | |

| | | | |
|---|---|---|---|
| Uterus, Cervix | Bone Marrow | Bone Marrow | Bone Marrow |

| | | | | | | | | | |
|---|---|---|---|---|---|---|---|---|---|
| E | epi | NA | ery | 0 | ery | 0-1 | ery | 2 | occ |
| | | | | | | [C] | | [N] | |
| | gland | 3 | mye | 2 | mye | 2 | mye | 2 | |
| | | [C] | | [N] | | [N] | | [N] | |
| | sm | 0 | mega | 0 | mega | 0 | mega | 0 | |
| | other | 0 | mac | 0-1 | mac | 1-2 | mac | 1-2 | |
| | | | | [C] | | [C] | | [C] | |

| | | | | | | |
|---|---|---|---|---|---|---|
| inucin | other - | 3 | other - | 3 | other - | 3 |
| | gran | [C] | gran | [C] | gran | [C] |

TABLE 12
Slide 2 - Isotype 2N9

| | 1 | | 2 | | | 3 | | 4 | | | 5 | | |
|---|---|---|---|---|---|---|---|---|---|---|---|---|---|
| | Pancreas | | Pancreas | | | Pancreas | | Placenta | | | Placenta | | |
| A | acini | 1-2 | acini | 2-3 | | acini | 2-3 | tropho | 0 | | tropho | 0 | |
| | | [C] | | [C] | | | [C] | | | | | | |
| | islet | 0 | islet | 0 | | islet | 0 | stroma | 0-1 | | stroma | 0-1 | |
| | | | | | | | | | [C] | | | [C] | |
| | duct | 0 | duct | 0 | | duct | 0 | other | 0 | | other | 0 | |
| | other - | 2 | other - | 0 | | other - | 0 | | | | | | |
| | mac, fib | [C] | mac, fib | | | mac, fib | | | | | | | |
| | Salivary Gland | | Salivary Gland | | | Salivary Gland | | Skin | | | Skin | | |
| B | epi | 0 | epi | 0-2 | | epi | 0 | epiderm | 3 | | epiderm | 3 | |
| | | | | [C] | | | | | [C] | | | [C] | |
| | other | 0 | other | 2 | rare | other | 0 | derm | 2 | rare | derm | 2 | rare |
| | | | | [C] | | | | (fib) | [C] | | (fib) | [C] | |
| | | | | | | | | other | 0 | | other | 0 | |
| | Spleen | | Spleen | | | Spleen | | Stomach | | | Stomach | | |
| C | lym | 0 | lym | 0 | | lym | 0 | epi | NA | | epi | 2 | rare |
| | | | | | | | | | | | | [C] | |
| | rd pulp | 0 | rd pulp | 0 | | rd pulp | 0 | sm | 0 | | sm | 0 | |
| | other- | 3 | other- | 3 | | other- | 3 | other | | | other | 2 | rare |
| | gran | [C] | gran | [C] | | gran | [C] | | | | | [C] | |
| | Thymus | | Thymus | | | Thymus | | Thyroid | | | Thyroid | | |
| D | lym | 0 | lym | 0 | | lym | 0 | follicle | 0 | | follicle | 0 | |
| | epi | 2 | epi | 2 | | epi | 2 | other - | 3 | rare | other - | 0 | |
| | | [C] | | [C] | | | [C] | fib, gran | [C] | | fib, gran | | |
| | other - | 3 | other - | 3 | | other - | 3 | | | | | | |
| | gran | [C] | gran | [C] | | gran | [C] | | | | | | |
| | Uterus | | Uterus | | | Uterus | | Uterus, Cervix | | | Uterus, Cervix | | |
| E | endo | NA | endo | 0 | | endo | 0 | epi | NA | | epi | NA | |
| | myo | 0 | myo | 0 | | myo | 0 | gland | NA | | gland | NA | |
| | other - | 3 | other - | 0 | | other - | 0 | sm | 0 | | sm | 0 | |
| | gran | [C] | gran | | | gran | | | | | | | |
| | | | | | | | | other | 0 | | other | 0 | |

| | 6 | | | 7 | | | 8 | | | 9 | | |
|---|---|---|---|---|---|---|---|---|---|---|---|---|
| | Placenta | | | Prostate | | | Prostate | | | Prostate | | |
| A | tropho | 0 | | gland | 0 | | gland | 0 | | gland | 0 | |
| | stroma | 0-2 | | stroma | 0 | | stroma | 0 | | stroma | 0-3 | |
| | | [C, M] | | | | | | | | | [C] | |
| | other | 0 | | other | o | | other | o | | other | o | |
| | Skin | | | Small Intestine | | | Small Intestine | | | Small Intestine | | |
| B | epiderm | 3 | | epi | 3 | | epi | 3 | | epi | 3 | |
| | | [C] | | | [C, M] | | | [C, M] | | | [C, M] | |
| | derm | 2 | rare | sm | 0-1 | | sm | 0 | | sm | 0 | |
| | (fib) | [C] | | | [C] | | | | | | | |
| | other | 0 | | inf | 3 | rare | inf | 3 | rare | inf | 3 | rare |
| | | | | | [C] | | | [C] | | | [C] | |
| | | | | gang | 0 | | gang | NA | | gang | 0 | |
| | | | | other | 0 | | other | 0 | | other | 0 | |
| | Stomach | | | Testis | | | Testis | | | Testis | | |
| C | epi | NA | | semi | 0 | | semi | 0 | | semi | 0 | |
| | sm | 0 | | leydig | 2 | rare | leydig | 0 | | leydig | 0 | |
| | | | | | [C] | | | | | | | |
| | other | 2 | occ | other | 0 | | other | 0 | | other | 0 | |
| | | [C] | | | | | | | | | | |
| | Thyroid | | | Tonsil | | | Tonsil | | | Tonsil | | |
| D | follicle | 0 | | epi | 0 | | epi | 0 | | epi | 0 | |
| | other - | 0 | | lym | 0 | | lym | 0 | | lym | 0 | |
| | fib, gran | | | | | | | | | | | |
| | | | | other - | 3 | rare | other - | 3 | rare | other - | 3 | rare |
| | | | | gran | [C] | | gran | [C] | | gran | [C] | |
| | Uterus, Cervix | | | Bone Marrow | | | Bone Marrow | | | Bone Marrow | | |
| E | epi | NA | | ery | 0 | | ery | 0-1 | | ery | 2 | occ |
| | | | | | | | | [C] | | | [N] | |
| | gland | 3 | | mye | 2 | | mye | 2 | | mye | 2 | |
| | | [C] | | | [N] | | | [N] | | | [N] | |
| | sm | 0 | | mega | 0 | | mega | 0 | | mega | 0 | |
| | other | 0 | | mac | 0-1 | | mac | 1-2 | | mac | 1-2 | |
| | | | | | [C] | | | [C] | | | [C] | |

| | | | | | | |
|---|---|---|---|---|---|---|
| mucin | other - | 3 | other - | 3 | other - | 3 |
| | gran | [C] | gran | [C] | gran | [C] |

Key for Tables 9-12:

C—cytoplasmic staining
M—membranous staining
N—nuclear staining
occ—occasional
SS—subset
Al mac—alveolar macrophages
Cx—cortex
Derm—dermis
Endo—endothelium
Epi—epithelium
Epiderm—epidermis
Ery—erythroid precursors
Fib—fibroblasts
Gang—ganglion cells or ganglia
Glom—glomeruli, glomerular
Gran—granulocyte
Hep—hepatocytes
Inf—inflammatory cells
Kupf—Kupffer cells
Leydig—Leydig cells
Lym—lymphocytes
Mac—macrophages
Med—medulla
Mega—megakaryocytes
Meso—mesothelium
Mye—myeloid cells, myeloid precursors
Myo—myocytes
Neu—neurons

Pneum—pneumocytes

Rd pulp—red pulp
RTE—renal tubular epithelium
Semi—seminiferous epithelium
Skel musc—skeletal muscle
Sm, sm musc—smooth muscle
Tropho—trophoblasts

### Other Embodiments

Although specific embodiments of the invention have been described herein for purposes of illustration, various modifications may be made without deviating from the spirit and scope of the invention. Accordingly, the invention is not limited except as by the appended claims.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed:

1. An isolated fully human monoclonal anti-cancer antibody wherein said monoclonal antibody is 1061_I16 (TCN-462), 1226_K16, 1242_P11, 1242_N12, 1256_B2, 1250_I13, 1252_B7, 1248_C17, 1247_A18, 1252_O13, 1038_D5 (TCN-445), or 1261_P5.

2. An isolated fully human monoclonal anti-cancer antibody or fragment thereof, wherein said antibody comprises:
(a) a V_{H} CDR1 region comprising the amino acid sequence of SEQ ID NO: 49, 57, 75, 87, 95, 101, 103 or 119;
(b) a V_{H} CDR2 region comprising the amino acid sequence of SEQ ID NO: 50, 59, 65, 71, 79, 88, 96, 104, 110 or 121; and
(c) a V_{H} CDR3 region comprising the amino acid sequence of SEQ ID NO: 51, 58, 64, 66, 72, 80, 82, 89, 97, 105, or 111.

3. The antibody of claim 2, wherein said antibody further comprises:
(a) a V_{L} CDR1 region comprising the amino acid sequence of SEQ ID NO: 60, 81, 90, 98, 106, 112, 115, 116, 117 or 118;
(b) a V_{L} CDR2 region comprising the amino acid sequence of SEQ ID NO: 53, 61, 67, 73, 76, 83, 91, 99, or 113; and
(c) a V_{L} CDR3 region comprising the amino acid sequence of SEQ ID NO: 52, 54, 62, 68, 77, 85, 92, 100, 107, or 114.

4. An isolated fully human monoclonal anti-cancer antibody or fragment thereof, wherein said antibody comprises:
(a) a V_{H} CDR1 region comprising the amino acid sequence of SEQ ID NO: 55, 63, 69, 84, 86, 93, 101, or 108;
(b) a V_{H} CDR2 region comprising the amino acid sequence of SEQ ID NO: 56, 65, 70, 74, 78, 94, 102, 109 or 120; and
(c) a V_{H} CDR3 region comprising the amino acid sequence of SEQ ID NO: 51, 58, 64, 66, 72, 80, 82, 89, 97, 105, or 111.

5. The antibody of claim 4, wherein said antibody further comprises:
(a) a V_{L} CDR1 region comprising the amino acid sequence of SEQ ID NO: 60, 81, 90, 98, 106, 112, 115, 116, 117 or 118;
(b) a V_{L} CDR2 region comprising the amino acid sequence of SEQ ID NO: 53, 61, 67, 73, 76, 83, 91, 99, or 113; and
(c) a V_{L} CDR3 region comprising the amino acid sequence of SEQ ID NO: 52, 54, 62, 68, 77, 85, 92, 100, 107, or 114.

6. An isolated fully human monoclonal anti-cancer antibody or fragment thereof comprising:
a) a heavy chain sequence comprising the amino acid sequence of SEQ ID NO: 1 and a light chain sequence comprising amino acid sequence SEQ ID NO: 3;
b) a heavy chain sequence comprising the amino acid sequence of SEQ ID NO: 5 and a light chain sequence comprising amino acid sequence SEQ ID NO: 7;
c) a heavy chain sequence comprising the amino acid sequence of SEQ ID NO: 9 and a light chain sequence comprising amino acid sequence SEQ ID NO: 11;
d) a heavy chain sequence comprising the amino acid sequence of SEQ ID NO: 13 and a light chain sequence comprising amino acid sequence SEQ ID NO: 15;
e) a heavy chain sequence comprising the amino acid sequence of SEQ ID NO: 17 and a light chain sequence comprising amino acid sequence SEQ ID NO: 19;
f) a heavy chain sequence comprising the amino acid sequence of SEQ ID NO: 21 and a light chain sequence comprising amino acid sequence SEQ ID NO: 23;
g) a heavy chain sequence comprising the amino acid sequence of SEQ ID NO: 25 and a light chain sequence comprising amino acid sequence SEQ ID NO: 27;
h) a heavy chain sequence comprising the amino acid sequence of SEQ ID NO: 29 and a light chain sequence comprising amino acid sequence SEQ ID NO: 31;
i) a heavy chain sequence comprising the amino acid sequence of SEQ ID NO: 33 and a light chain sequence comprising amino acid sequence SEQ ID NO: 35;
j) a heavy chain sequence comprising the amino acid sequence of SEQ ID NO: 37 and a light chain sequence comprising amino acid sequence SEQ ID NO: 39;
k) a heavy chain sequence comprising the amino acid sequence of SEQ ID NO: 41 and a light chain sequence comprising amino acid sequence SEQ ID NO: 43; or
l) a heavy chain sequence comprising the amino acid sequence of SEQ ID NO: 45 and a light chain sequence comprising amino acid sequence SEQ ID NO: 47.

7. An isolated anti-cancer antibody, wherein said antibody has a heavy chain with three CDRs comprising an amino acid sequence selected from the group consisting of the amino acid sequences of SGYYWS (SEQ ID NO: 49), EINHSGSTNYNPSLKS (SEQ ID NO: 50) and GGGRAGGSCCIRRPREYFQH (SEQ ID NO: 51), and a light chain with three CDRs that include an amino acid sequence selected from the group consisting of the amino acid sequences of KSSQSVLYSSNNKNYLA (SEQ ID NO: 115), WASTRES (SEQ ID NO: 53) and QQYYSTPPRT (SEQ ID NO: 54).

8. An isolated anti-cancer antibody, wherein said antibody has a heavy chain with three CDRs comprising an amino acid sequence selected from the group consisting of the amino acid sequences of GGSFSG (SEQ ID NO: 55), EINHSGSTN (SEQ ID NO: 56) and GGGRAGGSCCIRRPREYFQH (SEQ ID NO: 51) and a light chain with three CDRs that include an amino acid sequence selected from the group consisting of the amino acid sequences of KSSQSVLYSSNNKNYLA (SEQ ID NO: 115), WASTRES (SEQ ID NO: 53) and QQYYSTPPRT (SEQ ID NO: 54).

9. An isolated anti-cancer antibody, wherein said antibody has a heavy chain with three CDRs comprising an amino acid sequence selected from the group consisting of the amino acid sequences of GYFWT (SEQ ID NO: 57), EINHRRTTTSNPSLRS (SEQ ID NO: 59) and ITEAVGVTSFDY (SEQ ID NO: 58), and a light chain with three CDRs that include an amino acid sequence selected from the group consisting of the amino acid sequences of SGSTSNIGNNFVA (SEQ ID NO: 60), DNDKRPS (SEQ ID NO: 61) and GTWDSTLSRV (SEQ ID NO: 62).

10. An isolated anti-cancer antibody, wherein said antibody has a heavy chain with three CDRs comprising an amino acid sequence selected from the group consisting of the amino acid sequences of GGSLSG (SEQ ID NO: 63), EINHRRTTT (SEQ ID NO: 65) and ITEAVGVTSFDY (SEQ ID NO: 58), and a light chain with three CDRs that include an amino acid sequence selected from the group consisting of the amino acid sequences of SGSTSNIGNNFVA (SEQ ID NO: 60), DNDKRPS (SEQ ID NO: 61) and GTWDSTLSRV (SEQ ID NO: 62).

11. An isolated anti-cancer antibody, wherein said antibody has a heavy chain with three CDRs comprising an amino acid sequence selected from the group consisting of the amino acid sequences of GYFWT (SEQ ID NO: 57), EINHKGKTTYNPTLKS (SEQ ID NO: 65) and IVEAVGVTSFDS (SEQ ID NO: 66), and a light chain with three CDRs that include an amino acid sequence selected from the group consisting of the amino acid sequences of SGSTSNIGNNHVS (SEQ ID NO: 116), DNNKRPS (SEQ ID NO: 67) and GTWDTRLSRV (SEQ ID NO: 68).

12. An isolated anti-cancer antibody, wherein said antibody has a heavy chain with three CDRs comprising an amino acid sequence selected from the group consisting of the amino acid sequences of GGGSFSG (SEQ ID NO: 69), EINHKGKTT (SEQ ID NO: 70) and IVEAVGVTSFDS (SEQ ID NO: 66), and a light chain with three CDRs that include an amino acid sequence selected from the group consisting of the amino acid sequences of SGSTSNIGNNHVS (SEQ ID NO: 116), DNNKRPS (SEQ ID NO: 67) and GTWDTRLSRV (SEQ ID NO: 68).

13. An isolated anti-cancer antibody, wherein said antibody has a heavy chain with three CDRs comprising an amino acid sequence selected from the group consisting of the amino acid sequences of GYFWT (SEQ ID NO: 57), EINHRGSSSYNPSLRS (SEQ ID NO: 71) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DDDKRPS (SEQ ID NO: 73) and GTWDSSLSRV (SEQ ID NO: 52).

14. An isolated anti-cancer antibody, wherein said antibody has a heavy chain with three CDRs comprising an amino acid sequence selected from the group consisting of the amino acid sequences of GGSFSG (SEQ ID NO: 55), EINHRGSSS (SEQ ID NO: 74) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DDDKRPS (SEQ ID NO: 73) and GTWDSSLSRV (SEQ ID NO: 52).

15. An isolated anti-cancer antibody, wherein said antibody has a heavy chain with three CDRs comprising an amino acid sequence selected from the group consisting of the amino acid sequences of GYFWS (SEQ ID NO: 75), EINHRGSSTYKSSLKT (SEQ ID NO: 121) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DNDKRPS (SEQ ID NO: 61) and GTWDNNLSRV (SEQ ID NO: 77).

16. An isolated anti-cancer antibody, wherein said antibody has a heavy chain with three CDRs comprising an amino acid sequence selected from the group consisting of the amino acid sequences of GGSFSG (SEQ ID NO: 55), EINHRGSST (SEQ ID NO: 78) and ITEAVGVTSFDS (SEQ ID NO: 72), and a light chain with three CDRs that include an amino acid sequence selected from the group consisting of the amino acid sequences of SGSTSNIGNNYVS (SEQ ID NO: 117), DNDKRPS (SEQ ID NO: 61) and GTWDNNLSRV (SEQ ID NO: 77).

17. An isolated anti-cancer antibody, wherein said antibody has a heavy chain with three CDRs comprising an amino acid sequence selected from the group consisting of the amino acid sequences of GYFWT (SEQ ID NO: 57), EINHRGTSS (SEQ ID NO: 79) and ITEAVGFTSFDY (SEQ ID NO: 80), and a light chain with three CDRs that include an amino acid sequence selected from the group consisting of the amino acid sequences of SGSTSNIGSNYVS (SEQ ID NO: 118), DNDKRPS (SEQ ID NO: 61) and GTWDSSLSRV (SEQ ID NO: 52).

18. An isolated anti-cancer antibody, wherein said antibody has a heavy chain with three CDRs comprising an amino acid sequence selected from the group consisting of the amino acid sequences of GGSFSG (SEQ ID NO: 55), EINHRGTSS (SEQ ID NO: 79) and ITEAVGFTSFDY (SEQ ID NO: 80), and a light chain with three CDRs that include an amino acid sequence selected from the group consisting of the amino acid sequences of SGSTSNIGSNYVS (SEQ ID NO: 118), DNDKRPS (SEQ ID NO: 61) and GTWDSSLSRV (SEQ ID NO: 52).

19. An isolated anti-cancer antibody, wherein said antibody has a heavy chain with three CDRs comprising an amino acid sequence selected from the group consisting of the amino acid sequences of GYFWS (SEQ ID NO: 75), EINHSGSTNYNPSLKS (SEQ ID NO: 50) and GMVVAGTRSDAFDI (SEQ ID NO: 64), and a light chain with three CDRs that include an amino acid sequence selected from the group consisting of the amino acid sequences of SGSSSNIGINTVN (SEQ ID NO: 81), SNNQRPS (SEQ ID NO: 76) and AAWDDSLNEV (SEQ ID NO: 85).

20. An isolated anti-cancer antibody, wherein said antibody has a heavy chain with three CDRs comprising an amino acid sequence selected from the group consisting of the amino acid sequences of IGSFRG (SEQ ID NO: 86), EINHSGSTN (SEQ ID NO: 56) and GMVVAGTRSDAFDI (SEQ ID NO: 64), and a light chain with three CDRs that include an amino acid sequence selected from the group consisting of the amino acid sequences of SGSSSNIGINTVN (SEQ ID NO: 81), SNNQRPS (SEQ ID NO: 76) and AAWDDSLNEV (SEQ ID NO: 85).

21. An isolated anti-cancer antibody, wherein said antibody has a heavy chain with three CDRs comprising an amino acid sequence selected from the group consisting of the amino acid sequences of GYFWS (SEQ ID NO: 75), EINHSGSTNYNPSLKS (SEQ ID NO: 50) and GIVVAGTRSDAFDI (SEQ ID NO: 82), and a light chain with three CDRs that include an amino acid sequence selected from the group consisting of the amino acid sequences of SGSSSNIGINTVN (SEQ ID NO: 81), NNNQRPS (SEQ ID NO: 83) and AAWDDSLNEV (SEQ ID NO: 85).

22. An isolated anti-cancer antibody, wherein said antibody has a heavy chain with three CDRs comprising an amino acid sequence selected from the group consisting of the amino acid sequences of IGSFRG (SEQ ID NO: 86), EINHSGSTN (SEQ ID NO: 56) and GIVVAGTRSDAFDI (SEQ ID NO: 82), and a light chain with three CDRs that include an amino acid sequence selected from the group consisting of the amino acid sequences of SGSSSNIGINTVN (SEQ ID NO: 81), NNNQRPS (SEQ ID NO: 83) and AAWDDSLNEV (SEQ ID NO: 85).

23. An isolated anti-cancer antibody, wherein said antibody has a heavy chain with three CDRs comprising an amino acid sequence selected from the group consisting of the amino acid sequences of SYWMN (SEQ ID NO: 87), NINQDGTEKNYVDSVKG (SEQ ID NO: 88) and GVFQGAPHFVF (SEQ ID NO: 89), and a light chain with three CDRs that include an amino acid sequence selected from the group consisting of the amino acid sequences of RSSQSLLHGNGFNYLD (SEQ ID NO: 90), LGSDRAS (SEQ ID NO: 91) and MQSLRTPLT (SEQ ID NO: 92).

24. An isolated anti-cancer antibody, wherein said antibody has a heavy chain with three CDRs comprising an amino acid sequence selected from the group consisting of the amino acid sequences of EFTFGS (SEQ ID NO: 93), NINQDGTEKN (SEQ ID NO: 94) and GVFQGAPHFVF (SEQ ID NO: 89), and a light chain with three CDRs that include an amino acid sequence selected from the group consisting of the amino acid sequences of RSSQSLLHGNGFNYLD (SEQ ID NO: 90), LGSDRAS (SEQ ID NO: 91) and MQSLRTPLT (SEQ ID NO: 92).

25. An isolated anti-cancer antibody, wherein said antibody has a heavy chain with three CDRs comprising an amino acid sequence selected from the group consisting of the amino acid sequences of RYDIS (SEQ ID NO: 95), WMNPNSGNTGYAQKFQD (SEQ ID NO: 96) and LRVESLGRRFFYAYNGMDV (SEQ ID NO: 97), and a light chain with three CDRs that include an amino acid sequence selected from the group consisting of the amino acid sequences of QASQDISNYLN (SEQ ID NO: 98), DASNLET (SEQ ID NO: 99) and QQYNNVLFT (SEQ ID NO: 100).

26. An isolated anti-cancer antibody, wherein said antibody has a heavy chain with three CDRs comprising an amino acid sequence selected from the group consisting of the amino acid sequences of GYTFNR (SEQ ID NO: 101), WMNPNSGNTG (SEQ ID NO: 102) and LRVESLGRRFFYAYNGMDV (SEQ ID NO: 97), and a light chain with three CDRs that include an amino acid sequence selected from the group consisting of the amino acid sequences of QASQDISNYLN (SEQ ID NO: 98), DASNLET (SEQ ID NO: 99) and QQYNNVLFT (SEQ ID NO: 100).

27. An isolated anti-cancer antibody, wherein said antibody has a heavy chain with three CDRs comprising an amino acid sequence selected from the group consisting of the amino acid sequences of DFYFH (SEQ ID NO: 103), WINPRSGATNYAHKFRG (SEQ ID NO: 104) and DMRRENGYNFDGTFDY (SEQ ID NO: 105), and a light chain with three CDRs that include an amino acid sequence selected from the group consisting of the amino acid sequences of QASQDIKNYLN (SEQ ID NO: 106), DASNLET (SEQ ID NO: 99) and QRYDAFPLT (SEQ ID NO: 107).

28. An isolated anti-cancer antibody, wherein said antibody has a heavy chain with three CDRs comprising an amino acid sequence selected from the group consisting of the amino acid sequences of GYTFTD (SEQ ID NO: 108), WINPRSGATN (SEQ ID NO: 109) and DMRRENGYNFDGTFDY (SEQ ID NO: 105), and a light chain with three CDRs that include an amino acid sequence selected from the group consisting of the amino acid sequences of QASQDIKNYLN (SEQ ID NO: 106), DASNLET (SEQ ID NO: 99) and QRYDAFPLT (SEQ ID NO: 107).

29. An isolated anti-cancer antibody, wherein said antibody has a heavy chain with three CDRs comprising an amino acid sequence selected from the group consisting of the amino acid sequences of SYWMS (SEQ ID NO: 119), NIKQDGSEKYYVDSVKG (SEQ ID NO: 110) and DSEVAAAGTHFHY (SEQ ID NO: 111), and a light chain with three CDRs that include an amino acid sequence selected from the group consisting of the amino acid sequences of RASQSISTYLN (SEQ ID NO: 112), AASSLQS (SEQ ID NO: 113) and QQSYTALT (SEQ ID NO: 114).

30. An isolated anti-cancer antibody, wherein said antibody has a heavy chain with three CDRs comprising an amino acid sequence selected from the group consisting of the amino acid sequences of GFSFSS (SEQ ID NO: 84), NIKQDGSEKY (SEQ ID NO: 120) and DSEVAAAGTHFHY (SEQ ID NO: 111), and a light chain with three CDRs that include an amino acid sequence selected from the group consisting of the amino acid sequences of RASQSISTYLN (SEQ ID NO: 112), AASSLQS (SEQ ID NO: 113) and QQSYTALT (SEQ ID NO: 114).

31. An antibody that binds the same epitope as monoclonal antibody 1061_I16 (TCN-462), 1226_K16, 1242_P11, 1242_N12, 1256_B2, 1250_I13, 1252_B7, 1248_C17, 1247_A18, 1252_O13, 1038_D5 (TCN-445), or 1261_P5.

32. A pharmaceutical composition comprising any one of the antibodies according to any one of claims 1-31 and a pharmaceutical carrier.

33. The composition of claim 32, further comprising a second anti-cancer antibody or a chemotherapeutic agent.

34. A diagnostic kit comprising the antibody according to any one of claims 1-31.

35. A method of treating or alleviating a symptom of cancer comprising, administering to a subject the composition of claim 32 or 33.

36. The method of claim 35, wherein said cancer is breast cancer or ovarian cancer.

37. A method for determining the presence of cancer in a patient, comprising the steps of:
(a) contacting a biological sample obtained from the patient with the antibody according to any one of claims 1-31;
(b) detecting an amount of the antibody that binds to the biological sample; and
(c) comparing the amount of antibody that binds to the biological sample to a control value, and therefrom determining the presence or absence of cancer in the patient.


SEQUENCE LISTING (121 sequences)

SEQ ID NO: 1 - protein, 126 aa, Homo sapiens
QLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGGRAGGSCCIRRPREYFQHWGQGTLVTVSS

SEQ ID NO: 2 - DNA, 384 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggcgggggtagggctggtggtagctgctgtatccgacgcccccgagaatacttccagcactggggccagggcaccctggtcaccgtctcctca

SEQ ID NO: 3 - protein, 114 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPRTFGQGTKLEIK

SEQ ID NO: 4 - DNA, 342 bp, Homo sapiens
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaggacttttggccaggggaccaagctggagatcaaa

SEQ ID NO: 5 - protein, 120 aa, Homo sapiens
QVQLQQWGAGLLKPSETLSLTCAVYGGSLSGYFWTWIRQPPGRGLEWIGEINHRRTTTSNPSLRSRLTISLDTSKNQFSLKLSSVTAADTAVYYCARITEAVGVTSFDYWGQGILVTVSS

SEQ ID NO: 6 - DNA, 360 bp, Homo sapiens
caggtacagctacaacagtggggcgcaggactgttgaagccctcggagaccctgtccctcacctgcgctgtctatggtgggtccttaagtggttatttctggacttggatccgccagcccccagggaggggactggagtggattggggaaatcaatcacagacgaactaccacctccaacccgtctctcaggagtcgactaaccatatcactagacacgtccaagaaccagttttccctaaagctgagttctgtgaccgccgcggacacggctgtttattattgtgcgagaattacggaggcggtgggggttacctcgtttgactactggggccagggaatcctggtcaccgtctcgagc

SEQ ID NO: 7 - protein, 109 aa, Homo sapiens
QSVLTQPPSVSAAPGQKVNISCSGSTSNIGNNFVAWYQQLPQRAPKLLIYDNDKRPSGIPDRFSGSKSGASATLGITGLQSGDEAYYYCGTWDSTLSRVFGGGTKLTVL

SEQ ID NO: 8 - DNA, 327 bp, Homo sapiens
cagtctgtattgacgcagccgccctcagtgtctgcggccccaggacaaaaggtcaacatctcctgctctggaagcacctccaacattggaaataattttgtggcctggtaccagcaactcccacaaagagcccccaaactcctcatttacgacaatgataagcgaccctcagggattcctgaccgattctctggctccaagtccggcgcgtcagccaccctgggcatcaccggactccaaagtggggacgaggcctattattattgcggaacatgggacagtaccctgagtcgggtgttcggcggagggactaagctgaccgttcta

SEQ ID NO: 9 - protein, 120 aa, Homo sapiens
QVQLTQWGAGLLKPSETLSLTCAVGGGSFSGYFWTWIRQSPGRGLEWIGEINHKGKTTYNPTLKSRLSILVDTFKNQFSLKLRSVVAADTAVYYCARIVEAVGVTSFDSWGQGILVTVSS

SEQ ID NO: 10 - DNA, 360 bp, Homo sapiens
caggtgcaactaacacagtggggcgcaggactgttgaagccttcggagaccctgtccctcacctgcgctgtcggtggtgggtccttcagcggttacttctggacttggatccgccagtccccagggaggggcctggagtggattggagaaatcaatcacaaaggaaaaacaacctacaacccgaccctcaagagtcgactgagcatattggttgacaccttcaagaaccaattttccctcaaattgaggtctgtggtcgccgcggacacggctgtctattactgtgcgagaattgtggaggcagtgggagttacgtcctttgactcctggggccagggaatcctggtcaccgtctcgagc

SEQ ID NO: 11 - protein, 109 aa, Homo sapiens
QSVLTQPPSVSAAPGQKVTISCSGSTSNIGNNHVSWYQQLPQTAPKLLIYDNNKRPSGIPDRFSGSKSGASATLGITGLQTGDEAYYYCGTWDTRLSRVFGGGTKLTVL

SEQ ID NO: 12 - DNA, 327 bp, Homo sapiens
cagtctgtattgacgcagccgccctcagtgtctgcggccccaggacagaaggtcaccatctcctgctctggaagcacctccaacattggaaataatcatgtatcttggtaccaacaactcccacagacagcccccaaactcctcatttatgacaataataagcgaccctctgggattcctgaccgattctctggctccaagtctggcgcgtcagccaccctgggcatcaccggactccagactggcgacgaggcctattattactgcgggacgtgggataccaggttgagtcgggtgttcggcggaggaaccaaactgaccgttcta

SEQ ID NO: 13 - protein, 120 aa, Homo sapiens
QVQLQQWGAGLVKPSETLSVTCAVYGGSFSGYFWTWIRQPPGRGLEWIGEINHRGSSSYNPSLRSRLSISVDTSKNQFSLKMRSVTAADTAVYYCARITEAVGVTSFDSWGQGVLVTVSS

SEQ ID NO: 14 - DNA, 360 bp, Homo sapiens
caggtgcagctacaacagtggggcgcaggactggtgaagccttcggagaccctgtccgtcacctgcgctgtctatggtgggtccttcagtggttatttctggacttggatccgccagcccccagggaggggactggagtggattggagaaatcaatcacagaggaagttcctcctacaacccgtctctcaggagtcgactaagtatatcagtggacacgtccaagaaccagttttccctaaagatgaggtctgtgaccgccgcggacacggctgtttattactgtgcgaggattacggaggcggtgggagtgacctcctttgactcctggggccagggagtcctggtcaccgtctcgagc

SEQ ID NO: 15 - protein, 109 aa, Homo sapiens
QSVLTQPPSVSAAPGQKVTISCSGSTSNIGNNYVSWYQQLPQRAPKLLIYDDDKRPSGIPDRFSGSKSGASATLAITGLQTGDEAYYYCGTWDSSLSRVFGGGTKLTVL

SEQ ID NO: 16 - DNA, 327 bp, Homo sapiens
cagtctgtattgacgcagccgccctcagtgtctgcggccccaggacaaaaggtcaccatctcctgctctggaagcacctccaacattggaaataattatgtatcctggtaccagcaactcccacaaagagcccccaaactcctcatttatgacgatgataagcgaccctcagggattcctgaccgattctctggctccaagtctggcgcgtcggccaccctggccatcaccggactccagactggggacgaggcctattattattgcggaacatgggatagtagcctgagtcgggtgttcggcggagggaccaaactgaccgttcta

SEQ ID NO: 17 - protein, 120 aa, Homo sapiens
QVQLQQWGAGLLKPSETLSLTCAVYGGSFSGYFWSWIRQPPGRGLEWIGEINHRGSSTYKSSLKTRLTMSVDTSKNQFSIKLTSVTAADTAVYYCARITEAVGVTSFDSWGQGVLVTVSS

SEQ ID NO: 18 - DNA, 360 bp, Homo sapiens
caggtgcagctacaacagtggggcgcaggactgttgaagccttcggagaccctgtccctcacctgcgctgtctatggtgggtccttcagtggttatttctggagttggatccgccagcccccagggaggggactggaatggattggggaaatcaatcacagaggaagttccacctacaagtcgtctctcaagactcgactaaccatgtcagtagacacgtccaagaaccagttttccataaagctgacttctgtgaccgccgcggacacggctgtttattattgtgcgagaattacggaggcggtgggagttacctcctttgactcctggggccagggagtcctggtcaccgtctcgagc

SEQ ID NO: 19 - protein, 109 aa, Homo sapiens
QSVLTQPPSVSAAPGQKVTISCSGSTSNIGNNYVSWYQQLPQRAPRLLIYDNDKRPSGIPDRFSGSKSGASATLGITGLQTGDEAYYYCGTWDNNLSRVFGGGTKLTVL

SEQ ID NO: 20 - DNA, 327 bp, Homo sapiens
cagtctgtattgacgcagccgccctcagtgtctgcggccccaggacaaaaggtcaccatctcctgctctggaagcacctccaacattggaaataattatgtatcctggtaccagcaactcccacaaagagcccccagactcctcatttatgacaatgataagcgaccctcagggattcctgaccgattctctggctccaagtctggcgcgtcagccaccctgggcatcaccggactccagactggggacgaggcctattattattgcggaacatgggataataacctgagtcgggtgttcggcggagggaccaaactgaccgttcta

SEQ ID NO: 21 - protein, 120 aa, Homo sapiens
QVQLQQWGAGLLKPSETLSLTCAVYGGSFSGYFWTWVRQLPGRGLEWIGEINHRGTSSYNPSLRSRLTISVDTSKNQFSLKLSSVTAADTAVYYCARITEAVGFTSFDYWGQGILVTVSS

SEQ ID NO: 22 - DNA, 360 bp, Homo sapiens
caggtgcagctacaacagtggggcgcaggactgttgaagccttcggagaccctgtccctcacctgcgctgtctatggtgggtccttcagtggttatttctggacttgggtccgccagctcccagggaggggactggagtggattggagagatcaatcacagaggaacttcctcctacaacccgtctctcaggagtcgactaaccatatcagtagacacgtccaagaaccagttttccctaaaactgagttctgtgaccgccgcggacacggctgtttattattgtgcgagaattacggaggcggtgggctttacctcctttgactactggggccagggaatcctggtcaccgtctcgagc

SEQ ID NO: 23 - protein, 109 aa, Homo sapiens
QSVLTQPPSMSAAPGQKVTISCSGSTSNIGSNYVSWYQQLPQRAPKLLIYDNDKRPSGIPDRFSGSKSGASATLDITGLQTGDEAYYYCGTWDSSLSRVFGGGTKLTVL

SEQ ID NO: 24 - DNA, 327 bp, Homo sapiens
cagtctgtattgacgcagccgccctcaatgtctgcggccccaggacaaaaggtcaccatctcctgctctggaagcacctccaacattggaagtaattatgtatcctggtaccagcaactcccacaaagagcccccaaactcctcatttatgacaatgataagcgaccctcagggattcctgaccgattctctggctccaagtctggcgcgtcagccaccctggacatcaccggactccagactggggacgaggcctattattattgcggaacatgggatagtagcctgagtcgggtgttcggcggagggaccaaactgaccgttcta

SEQ ID NO: 25 - protein, 122 aa, Homo sapiens
QVQLQQWGTGLLKPSETLSRTCAVYIGSFRGYFWSWLRQPPGKGLEWIGEINHSGSTNYNPSLKSRVTISVDTSKNQFSLKLTSVTAADTAVYYCARGMVVAGTRSDAFDIWGQGTLVTVSS

SEQ ID NO: 26 - DNA, 366 bp, Homo sapiens
caggtgcagctacagcagtggggcacagggctgttgaagccttcggagaccctgtcccgcacctgcgctgtctatattgggtccttccgtggttacttctggagctggctccgccagcccccagggaaggggctggagtggattggggagatcaatcatagtggaagcaccaactacaacccgtccctcaagagtcgagtcaccatatcagtagacacgtccaagaaccagttctccctgaagctgacctctgtgaccgccgcggacacggctgtgtattactgtgcgagaggcatggtagtggctggaacccggagtgatgcttttgatatctggggccaagggacactggtcaccgtctcctca

SEQ ID NO: 27 - protein, 109 aa, Homo sapiens
QSVLTQPPSASGTPGQRVTISCSGSSSNIGINTVNWYQQLPGTAPRLVIYSNNQRPSGVPARFSGSKSGTSASLAINGLQAEDEADYYCAAWDDSLNEVFGGGTKLTVL

SEQ ID NO: 28 - DNA, 327 bp, Homo sapiens
cagagtgtgctgactcagccaccctcagcgtctgggacccccgggcagagggtcaccatctcttgttctggaagcagctccaacatcggaattaatactgtaaactggtaccaacaactcccaggaacggcccccagactcgtcatctatagcaataatcagcggccctcaggggtccctgcccgattttctggctccaagtctggcacctcagcctccctggccatcaatgggctccaggctgaggatgaggctgattattactgtgcagcatgggatgacagcctgaatgaagtattcggcggagggaccaagctgaccgttcta

SEQ ID NO: 29 - protein, 122 aa, Homo sapiens
QVQLQQWGAGLLKPSETLSRTCAVYIGSFRGYFWSWLRQPPGKGLEWIGEINHSGSTNYNPSLKSRVTMSVDTSKNQFSLKLTSVTAADTAVYYCARGIVVAGTRSDAFDIWGQGTLVTVSS

SEQ ID NO: 30 - DNA, 366 bp, Homo sapiens
caggtgcagctacagcagtggggcgcagggctgttgaagccttcggagaccctgtcccgcacctgcgctgtctatattgggtccttccgtggttacttctggagctggctccgccagcccccagggaaggggctggagtggattggggagatcaatcatagtggaagcaccaactacaacccgtccctcaagagtcgagtcaccatgtcagtagacacgtccaagaaccagttctccctgaagctgacctctgtgaccgccgcggacacggctgtgtattactgtgcgagaggcatagtcgtggctggtacccggagtgatgcttttgatatttggggccaagggacactggtcaccgtctcgagc

SEQ ID NO: 31 - protein, 109 aa, Homo sapiens
QSVLTQPPSASGTPGQRVTISCSGSSSNIGINTVNWYQQLPGTAPRLVIYNNNQRPSGVPARFSGSKSGTSASLAINGLQSEDEADYYCAAWDDSLNEVFGGGTKLTVL

SEQ ID NO: 32 - DNA, 327 bp, Homo sapiens
cagtctgtgctgactcagccaccctcagcgtctgggacccccgggcagagggtcaccatctcttgttctggaagcagctccaacatcggaataaacactgtaaattggtaccaacaactcccaggaacggcccccagactcgtcatctataacaataatcagcggccctcaggggtccctgcccgattctctggctccaagtctggcacctcagcctccctggccatcaatgggctccagtctgaggatgaggctgattattactgtgcagcatgggatgacagtctgaatgaagtattcggcggagggaccaagctgaccgttcta

SEQ ID NO: 33 - protein, 120 aa, Homo sapiens
EVRLVQSGGGLVQPGRSLRLSCAAFEFTFGSYWMNWVRQAPGKGLEWVANINQDGTEKNYVDSVKGRFTISRDNTKNSLYLEMDNLRADDTGIYYCARGVFQGAPHFVFWGQGALVTVSS

SEQ ID NO: 34 - DNA, 360 bp, Homo sapiens
gaggtgcgcctggtgcagtctgggggaggcttggtccagcctgggaggtccctgagactctcctgtgcagcctttgaattcacctttgggagttattggatgaactgggtccgccaggctccagggaaggggctggagtgggtggccaacataaaccaagatggaactgagaaaaactatgtggactctgtgaagggccgattcaccatctccagagacaacaccaagaattcactgtatctggaaatggacaacttgagagccgacgacacgggtatttattactgtgcgagaggtgttttccagggggccccacattttgtcttctggggccagggagccctggtcaccgtctcgagc

SEQ ID NO: 35 - protein, 112 aa, Homo sapiens
ETVLTQSPLSLAVTPGEPASISCRSSQSLLHGNGFNYLDWYLQKPGQSPQLLIYLGSDRASGVPDRFSGSGSGTDFTLKISRVETEDVGVYYCMQSLRTPLTFGQGTKLEIK

SEQ ID NO: 36 - DNA, 336 bp, Homo sapiens
gagactgtacttactcagtctccactttccctggccgtcacccctggagagccggcctccatctcctgcaggtctagtcagagcctcctacatggtaatggattcaactatttggattggtacctgcagaagccaggccagtctccacagctcctgatctacttgggttctgatcgggcctccggggtccctgacaggttcagtggcagtggatcaggcacagattttacactgaaaattagcagggtggagactgaggatgttggcgtttattactgcatgcaaagtctacgcactcctctaacttttggccaggggaccaaactggagatcaaa

SEQ ID NO: 37 - protein, 128 aa, Homo sapiens
QVQLVQSGAEVKKPGASVKVSCKASGYTFNRYDISWVRQATGQGLEWMGWMNPNSGNTGYAQKFQDRVTMTRNTSIRTAYMELSSLRSDDTAVYYCASLRVESLGRRFFYAYNGMDVWGQGTTVTVSS

SEQ ID NO: 38 - DNA, 384 bp, Homo sapiens
caggtgcagctggtgcagtctggggctgaggtgaagaagcctggggcctcagtgaaggtctcctgcaaggcttctggctacaccttcaataggtatgatatcagctgggtgcgacaggccactggacaagggcttgagtggatgggatggatgaatcctaacagtgggaacacagggtatgcacagaagttccaggacagagtcaccatgaccaggaacacctccataagaacagcctacatggagctgagcagtctgagatctgacgacacggccgtgtattactgtgcgagtcttagggtggaatcgctgggccgtcgttttttctacgcctacaatggtatggacgtctggggccaagggaccacggtcaccgtctcgagc

SEQ ID NO: 39 - protein, 107 aa, Homo sapiens
DIQMTQSPSSLSASVGDRVTISCQASQDISNYLNWYQQKPGKAPKLLIYDASNLETGVPSRFSGSGSGTDFTFTISSLQPEDIATYYCQQYNNVLFTFGGGTKVEIK

SEQ ID NO: 40 - DNA, 321 bp, Homo sapiens
gacatccagatgacccagtctccatcctccctgtctgcatctgtaggagacagagtcaccatctcttgccaggcgagtcaggacattagcaactatttaaattggtatcagcagaaaccagggaaagcccctaaactcctgatctacgatgcatccaatttggaaacaggggtcccatcaaggttcagtggaagtggatctgggacagattttactttcactatcagcagcctgcagcctgaagatattgcaacatattactgtcaacaatataataatgtccttttcactttcggcggggggaccaaggtggagatcaaa

SEQ ID NO: 41 - protein, 125 aa, Homo sapiens
QVQLVQSGAEVQKPGASMKVSCKASGYTFTDFYFHWVRQAPGQGLEWMGWINPRSGATNYAHKFRGRVTMTSDTSIKTIYMSLGWLRSGDTAVYYCARDMRRENGYNFDGTFDYWGQGTLVTVSS

SEQ ID NO: 42 - DNA, 375 bp, Homo sapiens
caggtgcaattggtgcagtctggggctgaggtacagaagcctggggcctcaatgaaagtctcctgcaaggcttctggatacaccttcaccgacttctattttcattgggtgcgacaggcccctggacaaggccttgagtggatgggatggatcaaccctagaagtggtgccacgaattatgcacataaatttcggggcagggtcactatgaccagtgacacgtccatcaaaacaatctacatgagtcttggttggctgagatctggtgacacggccgtatattactgtgcgcgggatatgagacgtgaaaacggttacaattttgatgggactttcgactactggggccagggaaccctagtcaccgtctcgagc

SEQ ID NO: 43 - protein, 107 aa, Homo sapiens
DIQMTQSPPSLSASVGDRVTITCQASQDIKNYLNWYQQKPGKAPKLLIYDASNLETGVPSRFSGSRSGTNFTFTINSLQPEDIATYFCQRYDAFPLTFGGGSKVEIK

SEQ ID NO: 44 - DNA, 321 bp, Homo sapiens
gacatccagatgacccagtctccaccctccctgtctgcatctgttggagacagagtcaccatcacttgccaggcgagtcaggacattaagaactatttaaattggtatcagcaaaaaccagggaaagcccctaaactcctgatctacgatgcatccaatttggaaacaggggtcccgtcaaggttcagtggaagtagatctgggacaaattttactttcaccatcaacagcctgcagcctgaggatattgcaacatatttctgtcaacggtatgacgctttcccgctcactttcggcggtgggtccaaggtggagatcaaa

SEQ ID NO: 45 - protein, 122 aa, Homo sapiens
EVQLVESGGGLVQPGGSLRLSCAASGFSFSSYWMSWVRQAPGKGLEWVANIKQDGSEKYYVDSVKGRFTISRDNAKNSLYLQMNSLRAEDTAVYYCARDSEVAAAGTHFHYWGQGTLVTVSS

SEQ ID NO: 46 - DNA, 366 bp, Homo sapiens
gaggtgcagctggtggagtctgggggaggcttggtccagcctggggggtccctgagactctcctgtgcagcctctggattcagctttagtagttattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggccgtatattactgtgcgagagattccgaagtggcagcagctggtacacactttcactactggggccagggaaccctggtcaccgtctcctca

SEQ ID NO: 47 - protein, 106 aa, Homo sapiens
DIQMTQSPSSLSTSVGDRVTITCRASQSISTYLNWYQQKPGKAPNLLIYAASSLQSGVPSRFSGSGSGTDFTLTISSLQPEDFATYYCQQSYTALTFGGGTKVEIK

SEQ ID NO: 48 - DNA, 318 bp, Homo sapiens
gacatccagatgacccagtctccatcctccctgtctacatctgtaggagacagagtcaccatcacttgccgggcaagtcagagcattagcacttatttaaattggtatcaacagaaaccagggaaagcccctaacctcctgatctatgctgcatccagtttgcaaagtggggtcccatcaaggttcagtggcagtggatctgggacagatttcactctcaccatcagcagtctgcaacctgaagattttgcaacttactactgtcaacagagttacactgcgctcactttcggcggagggaccaaggtggagatcaaa

SEQ ID NO: 49 - protein, 6 aa, Homo sapiens
SGYYWS

SEQ ID NO: 50 - protein, 16 aa, Homo sapiens
EINHSGSTNYNPSLKS

SEQ ID NO: 51 - protein, 20 aa, Homo sapiens
GGGRAGGSCCIRRPREYFQH

SEQ ID NO: 52 - protein, 10 aa, Homo sapiens
GTWDSSLSRV

SEQ ID NO: 53 - protein, 7 aa, Homo sapiens
WASTRES

SEQ ID NO: 54 - protein, 10 aa, Homo sapiens
QQYYSTPPRT

SEQ ID NO: 55 - protein, 6 aa, Homo sapiens
GGSFSG

SEQ ID NO: 56 - protein, 9 aa, Homo sapiens
EINHSGSTN

SEQ ID NO: 57 - protein, 5 aa, Homo sapiens
GYFWT

SEQ ID NO: 58 - protein, 12 aa, Homo sapiens
ITEAVGVTSFDY

SEQ ID NO: 59 - protein, 16 aa, Homo sapiens
EINHRRTTTSNPSLRS

SEQ ID NO: 60 - protein, 13 aa, Homo sapiens
SGSTSNIGNNFVA

SEQ ID NO: 61 - protein, 7 aa, Homo sapiens
DNDKRPS

SEQ ID NO: 62 - protein, 10 aa, Homo sapiens
GTWDSTLSRV

SEQ ID NO: 63 - protein, 6 aa, Homo sapiens
GGSLSG

SEQ ID NO: 64 - protein, 14 aa, Homo sapiens
GMVVAGTRSDAFDI

SEQ ID NO: 65 - protein, 9 aa, Homo sapiens
EINHRRTTT

SEQ ID NO: 66 - protein, 12 aa, Homo sapiens
IVEAVGVTSFDS

SEQ ID NO: 67 - protein, 7 aa, Homo sapiens
DNNKRPS

SEQ ID NO: 68 - protein, 10 aa, Homo sapiens
GTWDTRLSRV

SEQ ID NO: 69 - protein, 7 aa, Homo sapiens
GGGSFSG

SEQ ID NO: 70 - protein, 9 aa, Homo sapiens
EINHKGKTT

SEQ ID NO: 71 - protein, 16 aa, Homo sapiens
EINHRGSSSYNPSLRS

SEQ ID NO: 72 - protein, 12 aa, Homo sapiens
ITEAVGVTSFDS

SEQ ID NO: 73 - protein, 7 aa, Homo sapiens
DDDKRPS

SEQ ID NO: 74 - protein, 9 aa, Homo sapiens
EINHRGSSS

SEQ ID NO: 75 - protein, 5 aa, Homo sapiens
GYFWS

SEQ ID NO: 76 - protein, 7 aa, Homo sapiens
SNNQRPS

SEQ ID NO: 77 - protein, 10 aa, Homo sapiens
GTWDNNLSRV

SEQ ID NO: 78 - protein, 9 aa, Homo sapiens
EINHRGSST

SEQ ID NO: 79 - protein, 9 aa, Homo sapiens
EINHRGTSS

SEQ ID NO: 80 - protein, 12 aa, Homo sapiens
ITEAVGFTSFDY

SEQ ID NO: 81 - protein, 13 aa, Homo sapiens
SGSSSNIGINTVN

SEQ ID NO: 82 - protein, 14 aa, Homo sapiens
GIVVAGTRSDAFDI

SEQ ID NO: 83 - protein, 7 aa, Homo sapiens
NNNQRPS

SEQ ID NO: 84 - protein, 6 aa, Homo sapiens
GFSFSS

SEQ ID NO: 85 - protein, 10 aa, Homo sapiens
AAWDDSLNEV

SEQ ID NO: 86 - protein, 6 aa, Homo sapiens
IGSFRG

SEQ ID NO: 87 - protein, 5 aa, Homo sapiens
SYWMN

SEQ ID NO: 88 - protein, 17 aa, Homo sapiens
NINQDGTEKNYVDSVKG

SEQ ID NO: 89 - protein, 11 aa, Homo sapiens
GVFQGAPHFVF

SEQ ID NO: 90 - protein, 16 aa, Homo sapiens
RSSQSLLHGNGFNYLD

SEQ ID NO: 91 - protein, 7 aa, Homo sapiens
LGSDRAS

SEQ ID NO: 92 - protein, 9 aa, Homo sapiens
MQSLRTPLT

SEQ ID NO: 93 - protein, 6 aa, Homo sapiens
EFTFGS

SEQ ID NO: 94 - protein, 10 aa, Homo sapiens
NINQDGTEKN

SEQ ID NO: 95 - protein, 5 aa, Homo sapiens
RYDIS

SEQ ID NO: 96 - protein, 17 aa, Homo sapiens
WMNPNSGNTGYAQKFQD

SEQ ID NO: 97 - protein, 19 aa, Homo sapiens
LRVESLGRRFFYAYNGMDV

SEQ ID NO: 98 - protein, 11 aa, Homo sapiens
QASQDISNYLN

SEQ ID NO: 99 - protein, 7 aa, Homo sapiens
DASNLET

SEQ ID NO: 100 - protein, 9 aa, Homo sapiens
QQYNNVLFT

SEQ ID NO: 101 - protein, 6 aa, Homo sapiens
GYTFNR

SEQ ID NO: 102 - protein, 10 aa, Homo sapiens
WMNPNSGNTG

SEQ ID NO: 103 - protein, 5 aa, Homo sapiens
DFYFH

SEQ ID NO: 104 - protein, 17 aa, Homo sapiens
WINPRSGATNYAHKFRG

SEQ ID NO: 105 - protein, 16 aa, Homo sapiens
DMRRENGYNFDGTFDY

SEQ ID NO: 106 - protein, 11 aa, Homo sapiens
QASQDIKNYLN

SEQ ID NO: 107 - protein, 9 aa, Homo sapiens
QRYDAFPLT

SEQ ID NO: 108 - protein, 6 aa, Homo sapiens
GYTFTD

SEQ ID NO: 109 - protein, 10 aa, Homo sapiens
WINPRSGATN

SEQ ID NO: 110 - protein, 17 aa, Homo sapiens
NIKQDGSEKYYVDSVKG

SEQ ID NO: 111 - protein, 13 aa, Homo sapiens
DSEVAAAGTHFHY

SEQ ID NO: 112 - protein, 11 aa, Homo sapiens
RASQSISTYLN

SEQ ID NO: 113 - protein, 7 aa, Homo sapiens
AASSLQS

SEQ ID NO: 114 - protein, 8 aa, Homo sapiens
QQSYTALT

SEQ ID NO: 115 - protein, 17 aa, Homo sapiens
KSSQSVLYSSNNKNYLA

SEQ ID NO: 116 - protein, 13 aa, Homo sapiens
SGSTSNIGNNHVS

SEQ ID NO: 117 - protein, 13 aa, Homo sapiens
SGSTSNIGNNYVS

SEQ ID NO: 118 - protein, 13 aa, Homo sapiens
SGSTSNIGSNYVS

SEQ ID NO: 119 - protein, 5 aa, Homo sapiens
SYWMS

SEQ ID NO: 120 - protein, 10 aa, Homo sapiens
NIKQDGSEKY

SEQ ID NO: 121 - protein, 16 aa, Homo sapiens
EINHRGSSTYKSSLKT